SEQUENCE LISTING (125 sequences)

SEQ ID NO: 1 - protein, 255 aa, Burkholderia pseudomallei
MKKYLIEDLVTFDAHLMTLAHLDQTTELTTNESELLQMVLDGIATKAAVIERVWGSKGFVVTDSSYHQLVRSVRNRLEEHGISGALIKTLPRQGLRFLGSAQALTDAATPVTQPALADQGEPADEAALALLTQHEPQSAPPVGRARNWRRRLRFVERALLAGLVVWVGLVTWKMISINDRFPWLTGHFGVHTHAHVTPRSAGLKLLASIGVRPGPGERVYEISRNGDRWLAICAGTRHSSDERCTTYSTRDLTTS

SEQ ID NO: 2 - protein, 304 aa, Burkholderia pseudomallei
MRITSNQYHSIMTQVNRQATAGLMDTQIRMATEKRILRPSDDPTGAVRLAMLVRADAMLDQYRATGAMLNSRLQHSERVMSGIVDQLQNEAVKHLGFAMDGSRSPEDLSAYAQVLASVRDTLLQSANTRDSSGNYLFSGTAITTAPIRFDPAAPVGSRYTFEGNLEKQTAVVGDGVTEVANESLAEMADGLNALDEAIAKMSEPNANPNDPAYRDVLGRATDTIESVLASVNAKIGRMGSTQARLQMLDELHESAQIVGNQAALDVGKISPEEVFTDYQSYMVALQASQKLYAEVTQLTLLNVL

SEQ ID NO: 3 - protein, 452 aa, Burkholderia pseudomallei
MNIIYIGMSGIRAANQSLLAASQNTANIATGGYSRRGVLLSTGLTGGVDASGLLRLNESYKTQQLWQAAGQYGRYDAAQTYFDQLEALFGGNPVDGKTQANDLGFGKFIGALEAASGDPGDSVLRNGVIDAANSMTKQFNNMRSVLTNQLRSIEQQRVTTVEQVNGLAKTVAELNGKIAAGQASGADVSALMDQRDAAVDDLSRFAGVRVVQQANGSVDVSMANGQPLVVGAQAGKIELGRDAAGQQTLSLTFGSQVMPLRAASLGGELLGLAEFEEEVLRPQMEALRTLAVRTAELINDQLTQGFDMNGNPGKPLLTYDAATGLLGVDPSVKPEELGFSGDPANPGDNSNLLALVEIGSTSVDLPGIGDVSLGNAFATMMGKVGAASQQNKQAKGAAEAVRGQAELDWYSVSGVDRDEEVMNMLRFKDMYNANMKVIAVASELFETTLNSL

SEQ ID NO: 4 - protein, 116 aa, Burkholderia pseudomallei
MSNGTIGAGVRMAPAARMPGLAAQPAANESDERKAAYAAEQFEALFASQLLREMRNAIREISPEGSMFRDAVGSGLMDMMDGAIAQALATQRAFGIADFVLARIMPGGDPTGAAAR

SEQ ID NO: 5 - protein, 218 aa, Burkholderia pseudomallei
MMRILLALTGLACLGACASIEPPRPDFADDDLPPIATAPAAGRSGGVFNAVTAWSLASDGRAYRPGDTLTVTLEETTQASKRADTKFGKNGDMAVKPGVLFGSAIPFDSSFGAKRNFDGGGSSSQQNTLRGEITVIVNQVLAGGLLQVKGEKVLSLNQGEEVMRLSGYVRQADIDTNNRVSSRRLANARIKYVGKGALSDSNSAGWLTRFFNSPWMPF

SEQ ID NO: 6 - protein, 241 aa, Burkholderia pseudomallei
MDALIYTVMSGADRTLHGIAVHANNMANAQTDGFLAELEMAESQPVRGYGYDSRHLLNPRANALNGEAGVVVDTGRELDAAINGGGYFTVDAQGSEAYTRAGNFEIDADGALTVNGRPVIGEGGPIVLPEFTSLRIGEDGTISITSPGDTEVQAVERLALVDAPASALVKNEAGLLVARDGQPLPASNEVKVLGGHLERSNVSPVEEMIATMSLNRAFEVQMRMFNAAGEMADAGNRLIRG

SEQ ID NO: 7 - protein, 395 aa, Burkholderia pseudomallei
MSFNIALSGINAINSQLNTISHNIANANTYGFKAGRANFATMVAGTQANGTYIGSITQSVGTRGGFLPTGRALDGAIDGNGFFVTKDSDGSMLFTRFGYFQRDADGYIVDGFGRRAQGYGASGAYGDIRVPTDTEPAKASDSLEYVGNLSADWAAPKNPNFDKDDDTSFNHSVASTVYDSLGRQHIVTQYFVKGQPPSTDVIAYYAMDGEIVGGNTPVQTVLQFDTNGQLTAPNSPVDLDLGTPAGASALAIKVNYAGTTQVAGETTTTVNRDNGYAAGVPGEVMLDEDGGVVVQYSNGKQRKVGSLALATFANQDGLSAVGDTAWRASAASGNPLIGSAGSGALGKVVAGSLELSNADVTQELVDMMSAQRNYQANSKVLSTENQMMQALMQAL

SEQ ID NO: 8 - protein, 231 aa, Burkholderia pseudomallei
MSNTIQPRAGDMQQPGAGQHTPSPYANGANSPTDLFAKLLAAQIQNQNPLEPMDASQFVTQIMMGQQTEALGVVANLTQNSAALMESMLVVSLGSQVGTQVMVRTDSLELKDDTRDARIVLDNAESDVTVVLTDSAGNSHRIALGAQAKGDVNFTIDPEELGLPAGQYKIRVETASGAKAGVEIEGTLENVRLAADGKVILHVAGVGDVETSSISSFLGRRPANETRKETV

SEQ ID NO: 9 - protein, 124 aa, Burkholderia pseudomallei
MAFDLASGLGLHPTALKLRAERTKVIASNLANLDTPGYRARDLDFKRSLAEAGGDGAHARPGDALTGGVTLGYRVPYQAAQDGNTVELGIEQAAFAQNAVDFQMSLTFLNTKIKGLQTAITGNA

SEQ ID NO: 10 - protein, 141 aa, Burkholderia pseudomallei
MTRKEAFSRMLAGLEADRSRCEELRGLLEQQFDAALRRDGSLLMAAGSAIRALVQALGTSHEARRQCARVLAAPKRPASMSELCALLRGAARDALTLEWQSFDALMCECRRLNQRNATVLMSQFEIVQRVLHGESDTYVHP

SEQ ID NO: 11 - protein, 154 aa, Burkholderia pseudomallei
MKKKTVVSMIVGATLLVGGAIGGTWAFLSRNASHRPAPVAAQDKYKYVGESQIVVMLRQQDGQNATSIDAQAHYAMLDLVLKTTPEKEAIVRDHMLLLRSLTVDAVSQYTLEQLRQMNVAELANVLDRAFRQSYKTRAVAMPFESVMIGKLLAE

SEQ ID NO: 12 - protein, 109 aa, Burkholderia pseudomallei
MADFDVDRVGDLSAALTTRARRLADAAMQRDWASVTALDAELNAIVRRCAADGEPTRDMHAALQAVARVYERAAATIGDAHGELASRLVELAENRHAWIAYATIAEGGQ

SEQ ID NO: 13 - protein, 56 aa, Burkholderia pseudomallei
MTAGSEVVTNLAALYDYCVRRLNVAGIELNVEPLDEAAHLIGVLREGWQGAQDGGL

SEQ ID NO: 14 - protein, 441 aa, Burkholderia pseudomallei
MLRVTDPQAMAKTFAEAYMESARWQLERQANDVKRNKDALSGLGSLLTTFRDKQRELVAGGSMIQNTVTQSNDAFASVATTSGAKPGSYQFHVEQLATAHQLTIDVPSGIPAADAGSVKIKLADGSEFTVDLTAAKGEDGNVSAEELARVINSSSGASGNVHAAVIGGQLMLTSANTGADSKISFEIDSTSPLAGAFDGAVTISEAQNAKVKIGDPTNGKTIELQGNTLDSIEGLSITFRQVGSGTITVASDKEKTTESVKAFIDAYNELIDKLAELTRVGDAAKGDPGGPFSSDAGVRALVSQLRSELNKSVDGVKLYELGIDTDKKGKLVLDEKRFEQAFKASPDRFDKLFGDAQTGLVGNLGKALDGWLNEKNGQLKTRNESVAQAEKQLLAKQQEYDRRYQDVFERYTQEFTRLQVLQLQMDNTLDQIKNLFSTSKD

SEQ ID NO: 15 - protein, 145 aa, Burkholderia pseudomallei
MNEQRKVTRIAGWLAARQAEVDRLAADTARQEAVCQRYRRYLGAMQRLYDGSAAATAAALPMLAQNHAGYRQTLLDTLAQLRAALKVQEDNLEVSRALLAAKTLRCEVWRRELDRRRRALELARRRSEQRVQDELAVQAWRRTPA

SEQ ID NO: 16 - protein, 417 aa, Burkholderia pseudomallei
MTGRLTGVTGLLLKASGCRLETGERCVIESADGDRIDAQVVGFNREETFLMPFKKPVGLVAGAQVLSCERQGLLRIGRSWLGRIVNGLGEAIDGRGSLGGDAVLRAHPAPVNPLRKRPVRDVLDVGVRAINGLLTLGRGQRIGLMAGSGVGKSMLLGMITRATQADVVVVGLIGERGREVREFVDHALGEAGLAKSVLVAAPADESPLMRLKAAELCHAIAAHFRDTGANVLLLVDSLTRYAMAQREIALSVGEPPATRGYPPSVFGMLPQLVETAGNGEGAGSMSAIYTVLAEGDDQNDPVVDTARAILDGHIVLTRELAERGHYPAIDVPRSVSRCMSQVAGAEHGALARKVKEMIGRHEQIRDLIALGGYVQGADPATDEAVAKAARIDTFLRQDMLDVARHDATLAALKELIA

SEQ ID NO: 17 - protein, 195 aa, Burkholderia pseudomallei
MEDGYRAGFEHGEAEGLKQGRDAGFAAGAQQARDALRGEYAGIAKTLDAMRDAFARVQAEYLAARRTELVDVVAKVAKQVIRCELTLQPSQMAALIEETLETMPASGEAPEVHLSPDDHARLAGLLPERARHWRLVPDARLEAGECRVLLGGNEADAGCQHRLDACVERIGEQLPGAPSDVAAGATISDVQEEAS

SEQ ID NO: 18 - protein, 338 aa, Burkholderia pseudomallei
MMETPVADSNAQGSIEQAAIILLSIGEERAADVLRCLSRPELLRITQTMSSMAGVKVDVVKDALQRFFDQYREQSGIHGASRVYLQRALDLALGRDIANTVLNSVYGDSIRPKMARLQWVSPRWLADHIVHEHVRMQAVFLAFLPPAQASQVIEMLPPHTRDTILINVAKLDEVDHTLLVELEELIELCLKDLDLQGTTVEGVRQAAEIINRLPGDRMQMIELLRAHDPGVVTEIETSMYHFDMLARQSDNAIGRIVEVAPVEQWAIALKGADPAVLAALQRVMPRRQVQAFDDTIRRTGPLPSTRIEQVRRDIMARVKALADEGEIELRLVEEDVVS

SEQ ID NO: 19 - protein, 531 aa, Burkholderia pseudomallei
MPSPAALSRLLPLVVLAVALTAAATLYMRYDQANYKPLFGAREPVQLDDVVSTLDAQRIPYRIHPDTGQVLVARSDVARARMTLAARGVTAKAAPGLEQIDQDDPLGTSQFVQDVRFRRGLESELAQSIATLEPVERARVHLSIAKSSSFVMMDGDKSAASVVLTLKPGRRLAKEQIAAVINLVAGSVPNLAPAKVSVVDQHGAFLSARVDLDGDLTGTDEIAARFREDVTRNVEDLLAPSLGLGNFRVSVTALVNNDRVEETREQFGEAPKVMNEATRNEQNRDRVALGVPGSLSNRPVDVPASEPQADGGTQRAATTRQFAYDRNITQIKRGRGQLEKLSVAVVLNNAVSPAKGKPWSPEQLRRIDEILRSGLGIDKQRGDQLIVSSLDFAAPAAAEPWWKERDTLEQGGWYLTYALAALFGYLALARPMLRMLQQWVQHRYGAPAAAASASRLPAPEGGHAQAALPAADDARPPLAAMLPLLDDVELPPADSGVEVLIDHLRSLSEKEPERVAEVVKQWIRNDGNASS

SEQ ID NO: 20 - protein, 289 aa, Burkholderia pseudomallei
MLGRPTHLLDGFGATLRQYLDQFFTTSLGRRHGVAVAVRNVSFTSHVPELEARPWRRETVDRAAIGVHIERALLLLLLELRYGPAISPVMPPAAPADAGDTPPASDGETASAPPTPAETAATHAPPIPETTTERRLAERLAAELARTVARAIPFDAPAGEPPAPSSGAHLFAICSIEGAASRTVGEICFALDPVWQQRLFDHLKSSMSRARSVKRERPSPSTQLRLKLTAQLVEMSVPFGDVLRLRPGTVLPVRLHTPARVMTHDAQLFTASIAEHNGKLCLTSFVYVE

SEQ ID NO: 21 - protein, 115 aa, Burkholderia pseudomallei
MDSNEPNDLDALLDDLPADAPADTATAAHAPRRDPRQLLAKIPVQLTLEVGTATVSLAELMNIDEGTVLELDRLAGEPLLVKVNGTTVGHAEVVVSGENYGLKIIDLENLGDFAP

SEQ ID NO: 22 - protein, 255 aa, Burkholderia pseudomallei
MTRLRFRAARLATALTAAAALSAITATPALAEPLKLPLESLGLRGDYSVKTQILLFMTLLGLLPALAIVMTSFLRFAIVLALLRQALGLQQGLPARVITGVALLLTLLVMRPVGLDVWRHAIEPYDRDEITFREAIARAEQPVSRFMLAQTSKASLEQISRLSGERDVAKPEDHAFVVKAAAFMMSELKTAFQIGAMLFLPFLIIDLVVASTLMAMGMMMLSPLVISLPLKLLLFVLVDGWTLTVNTLTTSIQAY

SEQ ID NO: 23 - protein, 90 aa, Burkholderia pseudomallei
MLTSDLAVELVGNAIRLVTLMVAVIIAPGLLVGLVVALFQAATQINEQTLSFLPRLLATLAILVLAGHWLVQTLMDYCVATFTRAAQLVG

SEQ ID NO: 24 - protein, 258 aa, Burkholderia pseudomallei
MDAIVAAAAEWLARAWWPFCRILAALSAAPFIGESTVPARMRVTIALFLALIALPAVDPVAIPVFSARGALAAAEQILIGLLFGMLFHFVQAVFMMLGYLVASQMGLAMAVMNDPLNGATSDVVSGFMLVASILLFFAIDAHLVVVQVLYTSFRVWPVGTPIDLPSLKPLAYQAAWLFSTTMLLAAPLVFATVVLQIGKSYLSRATPSINLFSLGFAIMTVFGLLMLGAMLKGLPAHYASMTRHVLDVLEHHLGAARG

SEQ ID NO: 25 - protein, 335 aa, Burkholderia pseudomallei
MLVLLAPSWLAEFRALFLLELVDVTGDGALANAWSVALPVSIALFLKMIAPFFVVPLCVVLGSLLPGGWILSGAQLKPKFSRLNPMTNLGRLVSARHYGTFALSLLKALALIAMLAYLSATTLAAFLRLQHLPLQEALTRGFGLTLDGALALAAVMLVFALADVPFQRFVFLRGQRMTKQEVKDEHKNAEGRPEVRSRIRQLQRQAAQRVLSKTVPSADVVVVNPTHYAVALKYDTNRAEAPFVIAKGVDDMALAIRRIAQEHGVEVLSLPPLARAIYDTSQVNQQIPAALYRAVAQVLTYVLQLKAFRQGRRGLRPERPTDVAIPDFLDRTRPS

SEQ ID NO: 26 - protein, 696 aa, Burkholderia pseudomallei
MNKLTHLFADLRQYQYATPILLLAVLSMVILPLPPVILDLLFTFNIVLAIVVILVSVSVKRPLEFSVFPTIILATTLMRLTLNVASTRVVLLKGHEGTNAAGHVIEAFGKVVIGGNFVVGLVVFVILMIINFVVVTKGAERISEVSARFTLDALPGKQMAIDADLNAGLINQEQAQARRRDVTTEADFYGAMDGASKFVRGDAIAGILILLINMLGGLAIGVFAHGQAFGEAFQRYGLLTIGDGLVAQIPSLLLAAASAVIVTRVSDSGDFEEQVGRQMLASPNVLYSGAAVMMILAIIPGMPWLPFSAFAGVLGYVAWRVAKRPAPEREETQIHALETALRESNVQDLDWDSISHVHPLGIRVGYRLVDLVDPAHGAPLRRRVDGMRRTMSESMGILVPPITVRDDLSLPSSHYAIHLHGVEIDRAELHPDHLMAIPSPTTYGSVDGIPAADPAYGIPVTWIRPQDKAHAIGLGYQVVDGASVVATHVDKLLRDHLGELLTHDDVGSLLQRLGQVAPRLLEALEKAMPHNLIRKVCRVLLTDRVPLRNIVAIATALLENAEATQDPILLGAEVRCALRREIVAGIVGHRRSVPVFNLAGELEGILLAAFNQAQQSAKAPLDNFAIAPDLLTNLQSSLPGVQERMRTLGHAPILFVPPQLRPLLARYARVFAPGLHVLSYNEIPDERQIEIAGTLE

SEQ ID NO: 27 - protein, 349 aa, Burkholderia pseudomallei
MIRVLVIDDSATMRILLKKLIDKNEHMECVGVAPNPVAAQDLLREARPDVITLDIEMPKMNGLDFLDRIMRLMPVAVIMISTLTEAGSESALRALELGAIDFIAKPKLDFAEGVQAYAEEIYRKIETAGRAKVKKLTRDVPPVRMDAEPPAKPLLAEAGTEGRVVAVGASTGGTEAVKELLLGLPADCPPLLIAQHMPEPFMRSLAKRLDLLCAMRVKMAEDGETLRRGCAYIAPGHSNLTVEATAAGYACRIVRNAGEAQAGSSVDELFRSVAAAAGARAVGVVLTGSGSDGAAGARAMMAAGAFNIAQDAETSVVYSMPDAAIAACGINEVLPLEKIAGKLMELDRA

SEQ ID NO: 28 - protein, 344 aa, Burkholderia pseudomallei
MSMRTLLERIINGCDGMTCVGAAQDANAAREMIRALDPDVVTLDVEMPGMDGFEFLRRMMLLKPTPTIMVSGRTTSGSDAALRALELGAVDVIAKPVLTGPADLADYARDIAELIRGAAAARVKGGVLAAASPARGERACIQRLRGAKKVGIAATRLSRVIAIGASTGGTEALRAVLQDMSGTPPPILICQHMPEGFTASFAARLDTICGIRVKEAEQGEPLRYGCAYVAPGHSHLSLAATGRHYVCRLEASPPVNRHRPSVDVLFDSVARLAGKRALGAILTGMGKDGAAGLLRMRASGARTFAQDEQSCVVFGMPKEAIAMGAVDEILPLARMGARLREALQ

SEQ ID NO: 29 - protein, 200 aa, Burkholderia pseudomallei
MGVDGVGYHFYFDREFGRQAVRVNPGGFAIASADVMLATVLGSCVSVCMFDSAARVGGMNHFMLPGSGNGARHDSLSSLYGVNAMERLINGLLRRGALKCRLRAKLFGGGCVMPSLSDTRIGERNAAFVRAYLDAEGIRSVGGDMLGTRPRRVCYFPLTGRALCKRLVCGSDVADIATSERAYDGDLARRLPVAGSVELF

SEQ ID NO: 30 - protein, 273 aa, Burkholderia pseudomallei
MNSVDFEYTNADFRRIARHVYQRAGIVLTEAKRSLAYGRLVRRLRVLGIGRFSDYLDQLEADRSGDEWQHFVNALTTNLTSFFRESYHFDLLATQMRAASGPFRIWCAAASTGEEPYSLAMTACDARGTAANRVEIVASDIDTKVLAAAQAGIYKLHQLDKMPLRRKQAYFQRGSGSNGGTVRVKPALREMVTFRQINLLTESWPLDGSFDAVFCRNVLIYFDAPTQQKVLRNMARHLKSHGRLYVGHSENLRALDDLYAPCGRAVYELVNGR

SEQ ID NO: 31 - protein, 793 aa, Burkholderia pseudomallei
MTFNQKIISAFAVIIAGVIGMLGMEMWSLNNIESRVTELHDQRLATLTVMSSIEDDALKVSLGIAQAADYIWVKKVKAMSAGGVVGIGEEAVDRKSFESHVEGAERALARTIEKLEHNKINREGLSADEAAKIERIRVQLAQLQPDAVKDARRILDIAASGESDPDRYLAAYLSMRWQPFYEKVSELSRASTAATSKSITDTLDTVRERRRHVAVVSLFALVVLAGIVVWQIWYIKRSLGGSMEALSRHMTSIAHDDLSGDISVDERDKQSLMSSLAVMLERLRGAKAMVVENARIRVALDSGSANIMVVDDSRKVDFASHSLMRAFAAMQGALRREAPQFNSNQIVGALIDTVLERRVADELLGASSQQRCEVKVGGRDWLLVSSVIVDNGKRLGFVIEWIDRTNELQIEREVEAVIAAVAGGDFSSQVAEAGKTGFHQMLAQRLNRLTKTVADVVTEVATTFGKLADGDLRTRMAGAYQGVFAELKENCNRSIAQLERMAIRIRDGAHTINGAAREIASGNANLSSRTEQQAASLEQTAASMEELTSTVKQNAENAQQANTLALNASSIAQRGGQVVGQVVETMGRITASAKKVADITTVIDGIAFQTNILALNAAVEAARAGEQGRGFAVVASEVRNLAQRSAAAAKEIGALIGESVVETDSGAALVQSAGTTMDEIVNAVSRVVGIVGEISSACVEQSSGIEQVNQAVSHMDENTQQNAAMVEQAAAAAESLQEQAQLLHGTVEMFKVGERPAIDALPEVRAVEPPPAVPAVTRRRAGAASAADEWTDF

SEQ ID NO: 32 - protein, 89 aa, Burkholderia pseudomallei
MIIRASSRPRPSTYSARIAAPSAGAGRIWSRLNTTTSDTPSTTIPTVLPDTLRMIVTVVPPYCASPNPNFPRKSMTGTMLPRRLMTPLM

SEQ ID NO: 33 - protein, 742 aa, Burkholderia pseudomallei
MSIDLTQFSEVFFEEAEEHLRAMEALLLACDVSHPDAEDLNGIFRAAHSIKGGAGAFGFAELTGVTHVLETLLDRIRKKELPLCAAMVDAFLRATDLLAVMLAAYRSGEALPEQSIEATKRSLERLSQGEGAHGADAVMPDAEPVPASSEHDGDAHPLPEVLGDAYRKTSPAVMPPPGCLHVRIAAGAPIDDEGVMSCLASYGRVIEHYPGGCDQPWRYLCETSRDAADIHGTLTFLIDPDRLTIDQAAPEGGALPPEWEEEHDGYGFFVPVPTIGDGDAAHEAEARDEAGHARAETGRSDAQANASAGGSSAASAGVPALASPSPSRSRQAGDAAGGDSMAKRNGAGGSSIRVSVEKVDQLLNLVGELVITQSMLAQAAIELDANQRERLVGGIGQIERNSRELQEAVMSIRMLPISAVFNRFPRVVRDLAMQLGKQVELQMIGEHTELDKGFIEKLADPMMHLVRNSVDHGIEAPQVRVRRGKPATGRLTLRASQQGSDIAIEVIDDGGGLQRERILEKARESGLPVSESMTDREVWMLIFEPGFSTAAAVTDVSGRGVGMDVVRRNIQAMGGRIDVDSTPGVGTAISIKLPLTLAILDGMSVRIGGSRYIVPLAFITEAVMPADTDIRTVQRSGRVVNVRGEYLPLRSLADLFETGEDTTDGSLALILACDSQKVALLVDELLGQHQVVVKNLETNYRRVRGAAGATIMGDGRVALILDVMSLVMSARNSANVSEAESV

SEQ ID NO: 34 - protein, 66 aa, Burkholderia pseudomallei
MPHMDGISMLRAIRALPAHGATPIVVVTTEFGADLKQQGRDAGATGWMVKPIHPGKLVEMVRRLIG

SEQ ID NO: 35 - protein, 381 aa, Burkholderia pseudomallei
MTVKKKIRFLFQAASHWYCFASVYRAFGNDCRYDVEVVALPFLKNDVQLGEPSWAFLEAQGIPFVHYLQYDTDADAPDVVFLHCPYDETRPPAFHAAALHEHNVRLAYIPYGPDVGGGERNRRFQYGLDTQLLAWRIFARSEAHRMRYARNLPGSAKRVVVTGHPKLDELRGAPADNPFSPLVDGKCVLLWNPHFSVGFPAELRWSTFDRYVNAMCRLAAQRDDVAIIMRPHPNLFHTLMLREPGKRLIATLIAFANRHRNFVIDQSPLYKAAFDASHAMMTDTSSLMYEYLACDKPILHLDCDGGAGLNEEGRQLVRGMSRARAERDIVAFVDDIVRGRDVTQALRRRVIAGVLGECDGKNGERVKLAVDRSDLWQSVPA

SEQ ID NO: 36 - protein, 202 aa, Burkholderia pseudomallei
MSDTLICFDLDGTLSKQEILPRIAELADISEEIAALTQATIQGVIPFEMSFKLRVRLLRDICPRKISDYVAETVELDERILRYIRNGSGADCVVVTGNLDCWIEGLVRRIGVPYVSSLGEVKNGRLLGVANVLRKDAPVAQFRRDYRRIIAVGDGENDIPLFRQADVGIAYGGVHAPSANLANMADYIVYSSEALCDLLSMQ

SEQ ID NO: 37 - protein, 830 aa, Burkholderia pseudomallei
MNDPLRACSILPAVEVEARRSARVRRLLHWTAGLSIALAPFAAFHARAATLFTSEEQHWIDTHPVVRFAAGPGAQPFDYVEDGKHRGLTADYLDALARISGLEFERVDTTRWGSPTAAFAAHEIDLLPNVAYGLQNDVLQREATFSSPYLVGSAVIVTRESQTVIFDLDKLSGQTIAVRSGGAYERFLRQHHPSIRLLSTLEQRDMLDAVATGRADAAIGLDAMLVPAIRSSYSHRLYISGAVANMPAVIVMATRKDLPVLASIIDKSLNSLTARDVNELTTHWLYSSDFGAPSLRAITHYYTIEIVLGVFVVSGFAMLAFRSRAQYRLTAESEARKSRFIAVMSHEIRTPLNAIVSAIDLLSRAKLGPMEKQLADTAVKASASLVGMLDDVLDISKLHAKKLRLAHVASDIRTVAESAVIIMRAQADAKGLPLELHLDAPDHLDVYIDQTRIRQILVNLLSNAIKFTDAGRVCVDVKLAIADPPTHAPGELTLKVSDTGVGIPKNRQDHLFDAFEQADSTTTRRFGGTGLGLTICKELVALMGGTLALDSKERVGTSVTVVIPVDTAQREVCAPVDRDAVADSPSNARADRPRVLVVEDQPLNAELIRLQLAELHCDATIANTGQAALGLLDDQAFALVLLDCNLPDIDGYEVARRIRAARRNATTYLPVIAISALTDHAHLHECIAAGMDGSLRKPLMLDDVASIIDLWCRAAGVADGSAGSPGSSSPAERDIDLPRLASLFATTCAEDSALIGRALARGDFKRAASCAHRIKSAAHVMREHDIAQTAERLERALSTPDVNVNAARRNAAKLARLVSGIAPDRLSFDV

SEQ ID NO: 38 - protein, 236 aa, Burkholderia pseudomallei
MTTLDDCAAARTAYARAGAGAEQRWLVEYAPIVKRAARQLKSQAVGMLTLDDIEQIGLMGLLEALRRYGMPDAAFPCYAATRVRGAILDELRRHDWRPRSVRLEAHRTRDCERELCRSLGREPTEAEVSDALGISTTEYRERVMANNAEAMASFDSVLDDDALTHEESPEREIVRRRCLEQALARLNEREQRVIQLYYEFDLSLREIASVLGLTEARVCQINKAALKKMREVLSDA

SEQ ID NO: 39 - protein, 355 aa, Burkholderia pseudomallei
MELKTTMPAGGARAAAEAGLRGGIRTPAAMQPDIRDGRVRDASHAVVQMPPAGRGAADRRDAFAVHQRVNEQASGMQLVLDYLEQFGDRMKALKQALSARLAGGGSDLDVRAKIDSATAAWNRRAAATGGRLSETLELGGVDSARRAFVLRGLDAQDARRAEGEVITFHLGGTGARPATLYLAPDLTAREMAYRLKVGLAPLGIDCETHARERITLSVEESRWPVVARGLHVKGGGVRFPAGAPQRAAMEAVAGRIDASDWEAGDAGAMRAALTRTVQTVKRLDQVSILAEHALKRARQTLASMPSGLNAQWARDACERIAADASARGGYGQVVALTAVTAGLDRARVRALLSMR

SEQ ID NO: 40 - protein, 392 aa, Burkholderia pseudomallei
MDAILMKLRFRDHYSRRRTRAQRVCAFAAILSLLSVLLMAASRSSDAAPLGTLVSVEGVRDNQLVGYGLVVGLNGSGDGQQIRYTGQSIANVLKQFGVTLPEGIRLRSRNVAAVMVSANFPAGYVPGQKIDVTVSSMGDAKSLRGGTLLLTPLRAADGVVYALAQGNLVVPGVSAQGRSGSSVTINATAAGRIPQGATIEQEIPSDIDAKPFVRLSLKRPSFQTATSIVAAIDRMAGPGAATSRDGTSVEVRAPEDPTARVAFLAKLTAINVTPQKEPPRVVFNSRTGTVVISQGMTVSPAAVSHGTLKVTISEGAIVSQPNPLGGGKTAVVPLSQVDVQQDGNRMFNWPAGVSLQKIVDTINSTGASPDDVMAILQALDEAGALNGELVVI

SEQ ID NO: 41 - protein, 262 aa, Burkholderia pseudomallei
MNQAMWISKTGLQAQDAKLQAVANNLANVNTVGFKRDRVVFEDLFYRVERQPGAQLDQDNASPSGIQMGSGTRIVGTQKIFTDGSVQTTGQSLDVAVMGKGFIQVLMANGETGYTRAGQLSINAEGLLVNAQGLPLQPEISIPENAADIAIGENGIVAATMPGASSPSEVGQIQLANFVNPAGLLALGDNLYMETVASGPPNEGEPGTDVFGKIKQGALEGSNVQVVEEMVDMIAAQRTYEMNTKVLSAADNMLQQLSQAAR

SEQ ID NO: 42 - protein, 138 aa, Burkholderia pseudomallei
MSFKDIAQIAGSAMSAQTVRLNTIASNLANADSASGTEDATYRARKPVFASIFQLDAQGEPRGAAVQVLDVVKSTEPLRKVHQPGHPLADAEGNVLYPNVNPVEEMTDMLSASRAYSTNVEVLGRVNGMQLELLKLGQ

SEQ ID NO: 43 - protein, 155 aa, Burkholderia pseudomallei
MQRTAARSADRLVGAGHGVGQRSQYATRRAEIVRRDGLAGIEVARGNGNRCGDLGPGRELRHAAGCGAARRQGEPAELTRFADTDFERRIADGRGRLERRNDDRDIGVAQSGALREFREAGQRDLPSLLAKLGARRGGCGRRGYGRRGHGRRGRG

SEQ ID NO: 44 - protein, 264 aa, Burkholderia pseudomallei
MFIPEFDGRSSDWARAASAGPGVASWSAARGEFRAEFSRLQRDIAATIAQGFDTSGDGGRVRIQGSAPFAGANAIVRLLDGGSAHAAPEKAAFIERILPYANEAGKALSVSGELVAAHAALESGWGRRPLTTGEGANTHNLFGIKAGANWTGHVADVLTTEYVGGEPIKTVERFRAYPDYRSAFADYAALLRGSSRFREVIGTGDDAAAFAAALARGGYATDPAYSSKLRRIAADVAASLAAAGGISETRPALAGQSARPSPDR

SEQ ID NO: 45 - protein, 94 aa, Burkholderia pseudomallei
MRLFSTKYGVPAQRSNGASRASTTTHEGGARGAAAGDAGLEVELLRPAREVLRELPDFDAARVAALRAALQEGRLPFDAGKLAGLIQRFHGAKE

SEQ ID NO: 46 - protein, 383 aa, Burkholderia pseudomallei
MNAITAMTGPATDASGRAGAACTVLLGAGTVRGRPAGASDDEARTQGAESPPETDRLEDVSQSGAFAGVIAALEVQAPVRDVQATDDSARDSEDAARERVAAAAADAVTVGGTVRALRDGLGGLAFGQSPMIEAGKALRVPPARDGVRLPNEDPLRDGERLRDETGGARAGMRDSVSAAGAASAARVGQPAAGAPRDQQALSQVPGQPAADKPAAGAPGPQDGAIGAIGAPVDVPRAAARAYSPQSAPVRLGAELTRVLGERIDVQLQRGVERAVIRLDPQSMGTLHIAIRHEGGTVQIQMIASHEEVARQLQTMSEALRQDLSSRHHGDVTVVVRHGQGMEQGARHGDGGAHREEKRPGPALAEADVPDGDEGFHLGGRDVI

SEQ ID NO: 47 - protein, 110 aa, Burkholderia pseudomallei
MESIQQVSSQLRGRMAELAGQAASLSGAIVGQSGGSGEVNFTHMLGSMLKEVDDRQLDAAEKAVAVDSGRSDDVIGAVLSSQEASLSFSMLTQVRNKLSTALDDLLKMQI

SEQ ID NO: 48 - protein, 311 aa, Burkholderia pseudomallei
MSTNKESGHGPTIIRRAHKRHEEHSGGAWKVAFADFTLALMALFMVLWVMNVTPEKERKQVAAEIAGRPFFDGGVGIFEQRSRKPVVAPFDAQQPDRMRRRTSGAARERTIDSLQQRRALAARIMDEASRLGMDGNVATAINDDGVRITIHDSDKQGMFERRSDALNPPFIRLLTALVPVLGTVTNKMVIVGHTDATQYADASVFSNNWNLSSRRALRARQVLADGGMDAQRMFQISGMGDSVPAVPDNPNHDMNRRVELLLLTSRAEDTWRRLFRSEDVDVRPSADGIGLTVGQAQPGADRPAFASEPVR

SEQ ID NO: 49 - protein, 283 aa, Burkholderia pseudomallei
MQLAIGILLVFGCVFGGFVLHGGALSSIWQPTEILIISGAAAGALVMGNPRSVLSEMGSQIKHVIARKSNGVEFQRELLLMLYELLETVRDGGLKALDQHVEEPRESDLFRKYPMILAQPRLIAFIVDNFRLMAMGRITQHELEGMLDQELEAIESELMQPSRSLSKIAESMPGFGILAAVLGIVTTMANIGGSVSEIGLQVAAAMVGTFIGILLCYGVLEPLASAMVHRVKSDMSALECVKVVLCAQVAGKPTLLAVDAGRKLIQLDVKPSFSKLESWIMEI

SEQ ID NO: 50 - protein, 296 aa, Burkholderia pseudomallei
MLSLHTNIASMRIQGALGRTQQDLTTSMTRLGTGYRINSASDDAAGLQIATRLKSQHLGMGVAAQNTQNATSMLQTAEGAFKEVENILHRMNALATQAADSAWTQADRDSLQKEYDALGTELKNIVDNTSYGGEKLFAAGASGAPGGKLTEKLTFQVGNTSGEVMELDVSTALAAAVKAFEGASQVFGGGASGASGNTDITSISGANALLDNLSTALSAVGSLRAEIGAGQNRLTHIANNLTNMRDNTQDAEGRIRDVDYASETANMTKLNILQQAGGSMLKQASQMSQLVLSLLQ

SEQ ID NO: 51 - protein, 219 aa, Burkholderia pseudomallei
MRPVEHAVIPCAGVGSRLNRGIPKSLTEIDGHTLLGRTLLALEHIPHVFLVVGFRKELVISEALRYRRNLIFVPNEYFDRTNTQYSVKLASRLIRDDFLMVDGDVVFDPSSFAALLERFQACDGPAIAYSRRTTEDGVKVHLEGTRDETTCTGFFRGEGAHEWTGIGRLNNRVISGDSLFVYESIERHMPAPAYLVDSMDIDTPADLEIARATYGRHDR

SEQ ID NO: 52 - protein, 262 aa, Burkholderia pseudomallei
MTDAAANAKTSMNDECRIANRHVPIDYQATYRFFKGRAAGGADKVVQVLYQDDNPALAHARSEAECRILERALSFDGRSVLDIGCGTGRLADHIIDRVGYYFGFDLLAEFVETAKASLVARAIPESRYGFMALPCNARSIEMVTRRKRFDIVMMSGVSIYLNDEDFLGSLRCIAAAMHSGASLYLRDPIETADTRLTLNRFMSNQLSAEYSVIYRGADEYRRTVAEAFDASYDIGPLTPMYRDPALNGGWSTRQHYMVITRK

SEQ ID NO: 53 - protein, 311 aa, Burkholderia pseudomallei
MTEIGSYVPQFILRGKTRISQCQCASCRVRSPLVTHAWTNHVRQSALLQCETVSREALFERDAFELQTIPADVGQDAELDAWQNGVNQACIYLLLEQGLSIPERLHAIGVLLSRVSHAGDAQRDPALPIAVVEELIGLAGHGVLQERVAQMPIIEQYKRAYLRRLARVRMCLDLDLTSSMLLNLKLTELQVISDGFLADALHEFEQSRAVEKFFDKHDYVWQNYFLYRCFHEVFPGADPELYDLAFLDMCLDYFCLRSLCAILVSSQVDLDEDVVSSLFCAWQRNGKTSLGRDDHTDPLLIGFTLVGEVHD

SEQ ID NO: 54 - protein, 528 aa, Burkholderia pseudomallei
MRSAPNGGSRAVPRGVHGVDSDSIQFIGGFIVGHRAVARKNGRVSRLASKLLAQSPVVAVDAFPFVSVVCPTWQRRAFLPYLVYMFQYQDYPANRRELVILDDSAHSSQDLVEQLAAADPAGSVIRYYHVAERMTIGAKRNRLNELARGDYIVCMDDDDYYPPDKVSHAVRAMQERCATFSGSDRIYIWYSHIDRIYQTYSFGPHHALNGTFAYHRHYLADHRYDDTAVLAEESSFLGCFTASVLQIDPKRAILCVSHSANTFDKDFVLASCERAPFKLEDFVTDVRLLAHYRRLSHAPVSSRVEWRRFERIVINAGRDAGRLAAFHALLLELGVDERQIVEFRSASGDGADTQSHLDLAKLARANGWRNYLLLDDRLEFVRQEKAVANVNRFLRALDGFDWEVVLLGADVRSGVPLLTLPGAQRVNLAGEAVAYAVNQSYYASFIDHLERVLSVRGAGAQGAHERIDQAWLPLMASSRWLGLYPSFAYLSHREDGKDLIPGFFRKMRENADTPSGAPASFTPSRSTS

SEQ ID NO: 55 - protein, 248 aa, Burkholderia pseudomallei
MPNEKLPTPRAREQLSVIMLDDHEVVLSGITYQLKRERDIAVLGTFSRSRQLFDYLAQHGPALAHTRPVLLLDYALAPQDTDGIALIRRLCVQFPVCPILIVSGHHQPSIVRLALMAGARGYIGKNCTLEELVEAIRRVAGGERYVHPSLAERDDLVLPAAIEMRRGEAERATLASLLSPRELEVLRLTLQGFEVGQIAEKLSRSNKTISTQKRSAYRKLGISGDRELFKLERELLDSSGDAGSPYAN

SEQ ID NO: 56 - protein, 794 aa, Burkholderia pseudomallei
MYARRLVLSFSLLLGIAWGSNDALAVSSPSPSAAVEFDTQTLIARGYSADIAKFFSGDSRFLPGKQEVTIALNANQTYTTNVQFDDDGDPCFDAALLDTLRLRKPSDLAGCADPEQMWPEFHMKLHPGQFRIDMTVPEEAFDPRRRGNGLHSGGEAVLLNYDVFAQRFDTRFGSSHYLQARLEPGFNIGNWFFRNRGALTHSGKGFSYQLQDTYLARTVDAWGAITQIGQFTSTGENLGGLPMLGFQISSDRLQNSGQLTVPIQGIAESNATVEVRQRGRVVYRTIVPPGPFMLSDIGSVAQNADLQIEITEQDGRKRRFEMPVGMGYADAKQPTTWQAAIGRYQDAGNSDRTRAPAIFATGEISFSPLDNLRVSTAALAASGYLHASVQGTLSAKSGAWLASGVRYSRMAGAGQGYQFELQGSSRIGGNVFASASWLSRSIRYTTPDVALGSLGDATGTGQTKHSANASINWAHPRWGSFTYGLSYNSFVGAPNSVSHTLSIGRKIGRANVSLAAQISPKSGSSVYANVSIPLGGGSLSARTTRSQRGDLTFGTSYGNRLGRDGSYSIGASGSKSGQTANASVSMRTGFAQFGAGVAQSTSNSRSTYLSASGGIAYANGLFGTASSRIGETFAIVSVPNQKGLRISAPGGTSITNPFGTAVISSLTPYTKAQLRLDTQSLPMNVRLDTTTVDIGLRRGSVLTRTIHAAETRQLMLTIRDSTGATAPVGTTVLDEQGQFLGTIIGDGNFILNDDDIGRPIRLSGVNRSECRVTYEAPARFDPNQPYEEAEGTCG

SEQ ID NO: 57 - protein, 236 aa, Burkholderia pseudomallei
MNSLMLSHILFRSSRIKTFLASLFAACSLQSHAATFTLENPTIIFNETDQRVAFNVKNDGESPMLLISKLEDLGEDKLSQRFLIAPPIARIDPGQSQQINFSLKKGAKLEHEYMLKASFEGVSQTGRGSLVMPVRQEIGFLVQAGAIPVSKTPWDNLKLSVKDKQLTLSNPSAHVIRLAPSVKLLPTNKMVALAHAYLMPGESRTVEIDDATDKIEIVPLSRYGLVLPAVTMPVQH

SEQ ID NO: 58 - protein, 215 aa, Burkholderia pseudomallei
MKPKITFYAISIIIAQLWSANIQADTPNLDLMVKGTIDVPSCTVSVANNGIYDYGKILPTIITSGTGHTDLGKMTKAWSVNCDANTFMTFKVTDNRSESASVSSHDSRTLNTFGLGMVNKTGKLGYYTIKMTNPTVDGEATKLFNTLQTSDTDIYVRHSDIIQGWGSPNSLAIGRDFTADLTVNAYLAGTSIMAGPLTESIDLDGSTTLSFAFGL

SEQ ID NO: 59 - protein, 218 aa, Burkholderia pseudomallei
MKLKTSLLALSIAMLGTTAAHAAAPTQDLIVKGSIDVPACTVVAADNGVYDYGKISPTLIKSGTATTPLAKMEKAWTVNCDAETYMSFKVTDNRSDSASSGDNTAFGLGKVNETGKLGYYSVTMQDAQVDGKKARVYATPSTSLGGTGVVATYQKLNKLPSYTHGWANESSATQLAGKNFTANMVVEAVLAGTETMGGALKDAVDLDGSATLTFAFGL

SEQ ID NO: 60 - DNA, 747 bp, Burkholderia pseudomallei
atgcccaacgaaaagcttccaacgcccagggcccgcgagcagttgagtgtcatcatgctcgatgatcatgaggtggtactgagcggtatcacctatcaactgaagcgggagcgcgacatcgccgtactgggcacattctccagatcgcgtcagttattcgattatctggctcagcatgggcccgccttggcgcatacccggcctgtcctgcttctcgactatgcgctcgcgccgcaggacacggacggcatcgcactgattcggcgcttgtgcgtccagttccccgtttgcccgatcctcatcgtgtcggggcaccatcagccgtcgatcgtcagattggcgttgatggcgggcgcacgcggctatatcgggaagaactgcacgctggaggaactcgtggaggcgattcggcgggtggcgggcggcgaaagatatgtgcatccgtctctcgcggaacgcgacgacctggtgctgcctgccgcgatcgaaatgagacgcggagaggcggagcgcgcgacgctggcgtcgttgttgtcgccgcgcgaactggaggtgttgagactgactctgcagggtttcgaagtcggacagattgccgagaaactgagccgaagcaacaagacgatcagcacgcaaaagcgtagcgcataccgtaagctcggcatttccggcgatcgcgagctgttcaagctcgagcgggaattgctcgacagttcgggcgacgcgggcagtccgtacgcgaactga

SEQ ID NO: 61 - DNA, 2493 bp, Burkholderia pseudomallei
atgaacgatcccctgcgcgcctgctcgatcctgcccgccgtcgaggttgaagcccgtcggtctgctcgtgtacgccggttgcttcactggacggctggtctgagcatcgcgctcgcgccgtttgcggcgttccacgcacgcgcggccacgctgttcaccagcgaagagcagcactggatcgacacgcatcccgtcgtgcgtttcgcggccggccccggcgcgcagcccttcgactacgtggaagacggcaagcaccgcggccttaccgcggactatctcgacgcgctcgcccgcatcagcgggctggagttcgagagggtcgacaccactcgctggggctcgccgactgccgcgttcgccgcgcacgaaatcgacctgctgcccaacgtcgcctatggtctgcagaatgacgtccttcaacgcgaagcgaccttcagcagcccttatctcgtcggatcggcggtcatcgtgacacgcgaatcgcagaccgtgattttcgatctcgacaagctgtccggccaaaccatcgcagtgcgctcgggcggcgcgtacgaacgctttctccgtcagcaccatccgtcgatacgactactctcgaccctcgagcagcgcgatatgctcgacgcggtcgcgacgggccgcgcagacgcggcgatcgggctcgacgcgatgctcgtgcccgcgattcgcagcagttattctcaccgcctgtacatttcaggcgccgtcgcgaacatgcccgccgtgatcgtcatggccacgcgcaaagacttgccggtactggcgtcgatcatcgacaagtcgctgaactcgctgacagcgcgcgacgtcaacgaactgacgacgcactggctctacagctcggacttcggcgcgccgtctttgcgcgcgatcacgcactactacacgattgaaatcgtcctcggcgtcttcgtcgtgtcgggcttcgcgatgctcgccttccgatcccgtgcgcagtatcggttgacggccgagagcgaagcgcgcaagtcgcgcttcatcgcggtgatgagtcatgaaatccgcacgccgctcaacgcgatcgtatcggcaatcgacctgctcagccgcgcgaaactagggccgatggagaagcagctcgccgatacggcggtcaaggcgtccgccagtctcgtcggcatgctcgacgacgtgctggacatttccaagctccatgcgaaaaagctgcggctcgcgcacgtcgccagcgacatccggaccgttgccgagagcgccgtcatcatcatgcgcgcccaggccgacgccaagggcttgccgctcgaactgcatctcgacgcccccgaccatctcgacgtgtacatcgatcagacacgcatccgccagattctcgtcaacttgctgtcgaacgccatcaagttcacggacgccgggcgcgtgtgcgtcgacgtcaaactggcgatcgccgatccgccgacgcacgcgcccggagaactgacgctgaaggtgagcgacaccggcgtcggcattccgaagaaccggcaagaccacctgttcgacgcgttcgaacaggccgactcgacgacgacgcggcgcttcggcggtacgggcctcggtctgaccatctgcaaggaactcgtcgcattgatgggcggcacgttggcgctcgacagcaaggaacgcgtcggcacctcggtaacggtagtgattccggtcgacaccgcgcagcgcgaggtatgcgcgcccgtcgaccgggacgccgtcgcggattcgccatccaacgcacgcgctgaccggccgcgcgtgctggtggtggaagaccagccgctcaacgcggagctgatccgtcttcagcttgccgaactgcattgcgacgcgactatcgcgaacaccggccaagcggcgctcggtctgctggatgatcaggcgttcgcgctcgtgctgctcgactgcaatctgcccgacatcgacggttacgaagtagcgcgccgcattcgagcagcccggcggaatgcaacgacctatctgccggtcatcgccatttccgcactgacggaccatgcccatctgcatgagtgcatcgccgcgggcatggatggaagtttgcgcaagccgctcatgctcgacgatgtcgcgtcgataatcgacttgtggtgccgcgccgccggagtggccgacggctccgccggatcccccggctcatcgtcgcccgcggaacgggacatcgatctgcctcggctggcatcgctattcgcgaccacctgcgccgaggatagcgcgctcataggcagggccctcgcacgcggcgacttcaagcgagcggcgtcatgcgcgcatcgcatcaaaagcgccgcgcatgtgatgcgcgagcatgacatcgcgcaaaccgcggaacggctcgagcgcgcgctatcgactcccgatgtgaacgtcaacgcggcgcggcgcaacgcggcaaaactcgcccggctcgtctccggcattgccccggatcgcctctcgttcgatgtctga

SEQ ID NO: 62 - DNA, 1587 bp, Burkholderia pseudomallei
atgcgatcggcgccgaacggcggttcgcgcgccgtgccgcgcggggttcacggcgtggattccgactcgattcaattcatcggaggattcatcgtgggtcacagggccgtcgcccggaagaacggacgcgtttcgcgcctggcgtcgaagctgcttgcgcagtcgccggtggtggccgttgacgcgtttccgtttgtcagtgtcgtatgcccgacgtggcagcgcagagcattcctgccgtatctcgtctatatgttccagtatcaggactacccggcgaaccggcgcgagctcgtgattctcgacgactccgcgcacagcagccaggatctggtggagcaactggctgccgccgatcccgcgggttcggtgattcgctattaccacgtcgccgaacgaatgacgatcggcgccaagcgcaatcgcctgaatgaactcgcgcggggcgactatatcgtctgcatggatgacgacgactattatccgccggacaaggtgtcgcatgccgttcgtgcgatgcaggagcgttgtgcgacgttctccggcagcgaccgaatctacatctggtacagccatatcgaccggatctaccagacctattcgttcggaccgcatcacgcgttgaacgggacgttcgcctatcatcgccactacctggcggaccatcgctacgacgacacggccgtattggccgaagagagcagcttcctcggctgcttcacggcttcggtgctgcagatcgatccgaagcgggcgatcctgtgcgtttcgcacagcgcgaatacgttcgacaaggatttcgttctggcgagctgcgagcgcgccccgttcaagctcgaggatttcgtcaccgatgtgcgtctgctcgcgcattaccgtcgcctcagccatgcgcccgtgagctcgcgagtcgaatggcggcgcttcgagcggatcgtcatcaacgccggccgcgacgccggaaggctggcggcatttcacgcattgttgctcgagcttggcgtcgacgagcggcaaatcgtcgagttccgttctgccagcggggacggtgccgatacgcaaagccacctcgatttggcgaagctggctcgcgccaacggctggcgcaattatctcctgctcgacgaccggctcgagttcgttcgccaagagaaagcggtggcgaacgtcaatcgctttctacgcgcgctggatggcttcgactgggaagtcgtcctgctcggcgcggatgtgcgaagcggcgtcccgttgcttaccctcccgggcgcgcagcgggtcaatctcgccggcgaagcggttgcctatgcggtcaaccagtcttactacgcgagtttcatcgatcatctcgaacgagtgctttccgtgcgtggcgcaggcgcgcaaggcgcgcacgaacggatcgatcaggcatggcttccgctgatggcgagttcgcgctggctggggctgtacccgagtttcgcctatctctcgcatcgcgaagacggaaaggatctcattcccgggttcttcaggaagatgcgggagaacgcagacacgccgtccggagcacctgcttctttcactccatcccgatccacatcatga

SEQ ID NO: 63 - DNA, 936 bp, Burkholderia pseudomallei
atgaccgaaatcggaagctatgttccccagttcattttgcgcggcaaaacgcgcatcagccagtgccagtgcgcgagttgccgagtgcgatctccgctcgtgactcatgcatggaccaatcatgtgaggcagagcgcattgcttcagtgcgaaaccgtgtcgcgcgaggctctgtttgagcgcgacgcgttcgagctgcagacgattcccgcggacgtgggacaggatgccgagctcgatgcgtggcagaacggcgtgaatcaggcatgcatctatttactcctcgagcagggtctttcgattccggagcgactgcacgcgataggcgtgctgttaagccgggtatcgcacgccggcgacgcgcagcgcgatccggcgctgccgatcgccgtggtggaggaactgatcgggctggccggccacggtgtgctgcaggagcgcgtggcgcagatgccgatcatcgagcagtacaagcgcgcgtatcttcgtcgcctcgctcgcgtgagaatgtgtctcgatctcgacttgacgtcatcgatgttgctcaatctcaagctgacggaactgcaagtgatctcggatggctttctggctgacgcattgcacgagttcgagcaatcgcgggcagttgaaaagttcttcgataagcatgactacgtgtggcaaaactattttctgtacaggtgcttccacgaggtttttcccggcgcggatccggaactctatgatctggcattcctcgatatgtgtctcgactatttctgtctcagatcgctctgcgcaatactcgtatcgagccaagtcgatctggacgaagacgtcgtatcgagcctgttctgcgcatggcaacgcaacggcaagaccagcctgggccgcgacgatcacacggatccgctgctgatcggcttcacgttggtcggagaagtgcatgactga

SEQ ID NO: 64 - DNA, 789 bp, Burkholderia pseudomallei
atgactgatgcggcagcgaatgcgaagacgtcgatgaacgatgaatgccgtatcgcgaacagacatgtgccgatcgattatcaggctacctatcgctttttcaagggccgtgcggccggcggcgcggacaaagtggtgcaggtactgtatcaagacgacaatcccgcgcttgcgcatgcgcggtccgaggcggaatgtcggatattggagcgagccctgtctttcgacgggcgttcagtgctggatatcggttgcggcaccggccggctggcggatcacatcatcgatcgcgtcggttactacttcggctttgatctgcttgcggaatttgtcgagactgccaaggcctccctcgtcgcgcgggccatcccggaaagccgctatggtttcatggcgctgccatgcaacgcccgttcgatcgaaatggtcacccgccgtaagaggttcgatatcgtgatgatgtcgggcgtgtcgatatacctcaatgacgaggattttctagggagtcttcgttgcatcgcggcggcgatgcattccggcgcgagtctgtatctacgagatccgatcgagacggccgacacgcgcctcacgctcaatcggttcatgtcgaatcagttgtcggcggaatacagcgtgatctatcgcggtgccgatgagtatcggcgaacggtggccgaggcattcgacgcgtcgtacgacatcggaccgctgacgcctatgtatcgggaccctgcgctgaacggcggctggtcgacccggcagcactacatggtgattacccggaaatga

SEQ ID NO: 65 - DNA, 609 bp, Burkholderia pseudomallei
atgagcgatacgctgatttgcttcgatctggacggcacgctgagcaagcaggagattcttccgagaatcgccgaactggccgacatatcagaggaaattgcggcactcacgcaggcgacgattcaaggcgtgattccgttcgagatgtcgttcaagctgcgcgtacggctattgcgcgacatatgcccgagaaagatcagtgattacgtggccgagacggtcgagttggacgagcgcattctgcggtacatccggaatggaagcggggcggattgcgtggtcgtgacgggaaatctcgactgctggatcgaggggctcgtgcgacgcatcggcgtgccgtacgtcagttcgctgggcgaagtgaagaatgggcggctgctgggcgtcgcgaacgtattgcgcaaggatgcgcccgttgcgcaattccggcgtgactatcggcggatcatcgcggtcggagacggcgagaacgacattccgctgttcagacaggcggacgtgggtatcgcatatggcggcgtgcatgcgccatcggccaatctcgcgaacatggccgactacatcgtatattcgagcgaggcgctatgcgacctgttgagcatgcagtaa

SEQ ID NO: 66 - DNA, 660 bp, Burkholderia pseudomallei
atgcgacctgttgagcatgcagtaattccgtgcgcgggcgtcggctcgcgattgaatcgcggcataccgaaatcgttgacggaaatcgacggtcatacgttgctcgggcgcacgctgcttgcgctcgagcacattcctcatgtgtttctcgtggtcgggttccgcaaggaactggtcatcagcgaggcactgcgatatcgccgcaacctgatcttcgttccaaacgaatacttcgaccggacgaatacgcaatatagcgtcaagctggcgagccggctgatccgggacgatttcctgatggtcgacggcgatgtcgtgttcgacccgtcgtcgtttgccgcgctgctcgaacgatttcaggcctgcgatggccccgcgatcgcctacagcagaagaaccaccgaggacggcgtcaaagtccatctcgaaggaacgcgcgacgaaacgacatgcaccggattctttcgcggcgaaggcgcccatgaatggacgggcatcggccggctgaacaatcgcgtgatctcgggcgactcgctattcgtctacgagtcgatcgagcggcacatgcccgcgccagcctacctggtggattcgatggatatcgatacgcctgccgatttggaaatcgcgcgagcgacgtatggccgtcatgaccgttaa

SEQ ID NO: 67 - DNA, 1146 bp, Burkholderia pseudomallei
atgaccgttaagaagaagatccgctttctctttcaggcggcgagccattggtactgctttgcctccgtataccgggcattcggcaacgattgccgttacgacgtcgaagtcgtcgcgttgccctttctgaagaatgacgttcagctcggcgagccgtcgtgggcatttctcgaggcgcaggggattccgtttgtccattaccttcaatacgacacggatgcggacgcgcccgatgtcgtattcctgcattgtccgtacgacgagacgaggccgccggcatttcatgcggccgcgttgcatgagcacaacgtgcggctcgcgtatatcccttacggccccgatgtcggcggcggcgagcgaaatcgccggttccagtacgggctcgatacacaattgctcgcatggcgcatcttcgcacgctcggaagcgcatcggatgcgatatgcgcgcaacttgccgggcagtgcgaagcgcgtcgtcgtcaccgggcatccgaagctcgatgagcttcgcggcgcgcccgccgacaacccgttttcgccgctggtggacggcaagtgcgtattgctttggaatccgcatttctctgtgggctttccggcggagctgcgttggtcgacgttcgatcgctatgtgaacgcgatgtgccggctcgccgcgcagcgcgacgacgtcgcgatcatcatgcggccgcatccgaacctgttccatacgctgatgctgcgtgagccgggaaagcggctgatcgcgacgttgatcgcgttcgcgaatcggcatcgcaatttcgtgatcgatcaatctccgctgtacaaagcggctttcgatgcatcgcatgcgatgatgacagacacaagttcgctcatgtacgagtacctcgcctgtgacaagccgattctgcatctcgattgcgacggcggcgcgggactgaacgaggagggcaggcaactggttcgcgggatgtcgcgcgcgcgtgcggaacgcgacatcgttgcattcgtcgacgatatcgtgcgcggccgcgacgtcacgcaggcattgcgccggcgcgtgatcgccggcgtactcggcgagtgcgacggcaagaatggtgaacgcgtgaagcttgccgtcgatcgttcggatctctggcaatccgtgcctgcttga

SEQ ID NO: 68 - DNA, 891 bp, Burkholderia pseudomallei
atgcttagtctgcacacgaacatcgcttcgatgcgaatccaaggcgccctcgggcgcactcagcaggacctcaccacgtcgatgacgcgcctcggtacgggctaccgtatcaactcggcatccgacgacgccgccggcctccagatcgcgacgcgcctgaagtcgcagcacctcggcatgggcgtcgcagctcagaacacgcagaacgcaacgtcgatgctgcagacggcggaaggcgcgttcaaggaagtggagaacatcctgcaccgcatgaacgccctcgcgacgcaggcggccgactcggcatggacgcaggctgaccgtgactcgctgcaaaaggagtacgacgcgctcggcacggaactgaagaacatcgtcgacaacacgtcgtacggcggcgagaagctgtttgccgccggcgcctccggcgcgccgggggggaagctgacggaaaagctgacgttccaggtcggcaatacgtcgggcgaagtgatggagctggacgtgtcgacggcgctcgcggcggccgtaaaggctttcgagggtgcgtcccaggtgttcggcggcggcgcgtccggcgcgtcgggtaacaccgacatcacgagcattagcggcgcgaatgcgctgctcgacaatctgtccaccgcgctgtcggcggtgggcagcctgcgtgccgagatcggcgcgggccagaaccgcctgacgcacatcgcgaacaacctgacgaacatgcgcgacaacacgcaggacgcggaaggccgcattcgtgacgtcgattacgcgagcgaaaccgcgaacatgaccaagctgaacattctgcagcaagccggcggctcgatgctcaagcaggcgagccagatgagccagctcgtcctgtcgctcctgcagtaa

SEQ ID NO: 69 - DNA, 852 bp, Burkholderia pseudomallei
atgcaattggcaattgggatcctactggtattcggctgcgtgttcggcggattcgtgctgcatggcggcgcgttgagcagcatctggcaaccgaccgaaatcctgattatttccggtgcggccgcgggcgcgctggtgatgggcaatccgcgcagcgtgctgtccgaaatgggctcgcagatcaagcacgtgatcgcccgcaagagcaatggcgtcgaattccagcgcgagttgttgctgatgttgtacgaattgctcgagacggtgcgcgacggcggcctgaaggcgctcgatcagcatgtcgaggagcccagggaaagcgatctgttccgcaagtacccgatgattctcgcgcaaccgcgtctgattgcattcatcgtcgacaatttccggctgatggcgatggggcgcatcacgcagcatgagctcgaagggatgctggatcaggagctcgaggcgatcgagagtgagttgatgcagccgtcgcgctcgctgtcgaaaatcgcggaatcgatgccgggcttcggcattctcgcggccgtgctcggcattgtcacgacgatggccaacatcggcgggagcgtgtcggagatcggtctgcaggtggccgccgcgatggtcggcacattcatcggcatcctgctgtgctacggcgtgctggagccgctggccagcgcgatggtgcatcgcgtgaagagcgacatgtcggcgctggagtgcgtgaaggtggtgctgtgcgcgcaagtcgcgggcaagccgacacttctcgccgtcgatgcggggcgcaagctgatccagctggacgtcaagccgagcttctcgaagctcgagtcctggatcatggaaatctga

SEQ ID NO: 70 - DNA, 936 bp, Burkholderia pseudomallei
atgagcacgaacaaggaatcggggcacgggccgacgatcatcaggcgagcccacaagcgccacgaggagcacagtggtggagcgtggaaagtcgctttcgccgacttcacgttggcgttgatggcgctgttcatggtgctgtgggtgatgaacgtcacgccggagaaggagcgcaagcaagtggccgccgaaatcgcgggacggccttttttcgacggaggcgtcggcatcttcgagcagcgcagccgcaagccggtcgtcgcgccgttcgatgcgcagcagcccgatcggatgaggcgccggacatccggcgcggcgcgcgagcgcacgatcgattcgcttcagcagcgccgcgcgctcgccgcgcgcatcatggacgaggcgagcaggctcggcatggacggcaacgtcgccaccgcgatcaacgacgacggcgttcgcatcacgattcacgattccgacaagcagggaatgttcgagcggcgcagcgatgcactgaatcccccgttcatcaggctgctcacggcgctcgtgcccgtgctcggaaccgtgacgaacaagatggtgatcgtcgggcataccgatgcgacgcaatacgccgacgcctccgtgttcagcaacaactggaatctgtcgagccgccgcgcgctgcgcgctcgccaggttctcgccgacggcggcatggacgcgcagcgcatgtttcagatctccggcatgggcgacagcgtgccggccgtgccggacaatccgaatcacgacatgaaccggcgggtcgaactgctgctgctcaccagccgcgccgaagacacgtggcgcagattgtttcgcagcgaggatgtcgacgtccgaccgagtgcggacggaatcggcttgacggtggggcaggcgcagcccggcgcggatcggcccgcgtttgccagcgagccggttcgctga

SEQ ID NO: 71 - DNA, 201 bp, Burkholderia pseudomallei
atgccgcacatggacggcatcagcatgttgcgggcgattcgggcgctgccggcgcatggcgcgacacccatcgtcgtcgtcacgacggagttcggcgccgatctcaagcagcaagggcgcgatgcgggcgccacgggctggatggtcaagccgatccatcccgggaaactggtcgagatggtccgccggttgatcggttga

SEQ ID NO: 72 - DNA, 2229 bp, Burkholderia pseudomallei
atgtcgatagatttgactcaattcagcgaggtgttcttcgaggaagcggaggaacacctgcgcgcgatggaagcgttgctgctcgcgtgcgacgtcagccatcccgatgccgaggatctcaatgggatcttccgggcggcacattcgatcaagggcggagcgggggcgttcggcttcgccgaattgaccggcgtcacgcatgtgctcgaaacgctgctggaccggatcagaaagaaggagctgccgctgtgcgcggcgatggtcgacgcgtttttgcgcgccacggatctgcttgcggtgatgctcgcggcgtatcgcagcggagaggcgctgcccgagcagagtatcgaggcgacgaagcgcagcctcgagcgcctgagccaaggcgaaggcgcgcacggcgccgacgccgtcatgcccgatgcggaacccgtgccggcctcgtccgagcatgacggcgacgcacatccgctcccggaagttctcggcgacgcatatcggaaaaccagtccggcagtgatgccgccgcccggctgcctgcacgtgcggatcgcggccggcgctccgatcgacgacgaaggcgtaatgtcttgtctcgcgagctatggccgagtgatcgagcattatccgggaggctgcgatcagccgtggcgttacttgtgcgagacttcccgcgacgcagccgacattcacggcacgctgacgttcctgatcgatccggaccgcctgaccattgatcaagccgcgcccgaaggaggcgcgctgccgccggagtgggaggaggagcatgacggctatggcttcttcgtgcccgtgccgacgatcggcgacggcgacgcggcacatgaagccgaggcgcgcgatgaagccggccacgcgcgcgccgagactgggcgctccgatgcgcaagcgaacgcatccgcgggcggttcgtcggctgcatcggctggggtgccggcgctcgcttcgccgtcgccttcccgatcgaggcaagcgggcgacgccgccggcggcgacagcatggccaagcgcaacggcgcgggcggctcgtcgattcgcgtcagtgtcgagaaagtcgatcagttgctcaatctggtcggcgaactggtgatcacgcaatcgatgctcgcacaggcggcgatcgagctggatgcgaatcagcgcgagcggctggtcggcgggatcgggcagatcgaacggaattcgcgcgagttgcaggaagccgtgatgtcgatacggatgctgccgatctccgcggtgttcaaccgcttccctcgggtcgtgcgtgatctggccatgcagctcggcaagcaggtcgaactgcagatgatcggcgagcacaccgagctggacaagggattcatcgagaaactcgcggacccgatgatgcatctcgtgcgcaacagcgtcgatcacggtatcgaggcaccgcaagtgcgtgtgcgccgcggcaagcccgcgacaggccggctcacgctgcgcgcgtcgcagcaaggcagcgacatcgcgatcgaggtgatcgacgacggcggcggcctccagcgcgaacgcattctcgagaaggcgcgggaaagcggtctgccggtatcggaatcgatgaccgatcgcgaagtctggatgctgatcttcgagccggggttctcgaccgccgcagccgtgacggacgtgtcgggcagaggcgtcggcatggatgtcgtgcgccggaacattcaggcgatgggcgggcggatcgacgtggattcgacgccgggcgtgggcaccgcgatctcgatcaagttgcctctcacactggcgattctggacggcatgtcggtgcggatcggcggctcgcgctacatcgtgccgctcgcattcatcaccgaggccgtcatgcccgcggatacggacatccgcacggttcagcgcagcggacgcgtcgtcaacgtgcgcggcgaatacctgccgctgcgctcgctcgccgatctcttcgagacgggagaagacaccaccgacggcagcctggcgctgattctcgcgtgcgactcgcaaaaggtcgcgctgctcgtcgacgaacttctggggcagcaccaggtagtggtcaagaacctcgagaccaactaccggcgcgtgcgcggggcggcgggcgcgacgatcatgggagatggtcgcgtcgcgctgattctcgacgtcatgagtctcgtcatgtcggcgcggaattcggcaaacgtttcggaagcggaaagcgtgtag

SEQ ID NO: 73 - DNA, 270 bp, Burkholderia pseudomallei
atgatcatccgcgcgtccagcaggcccagaccgtcgacgtactccgcccggatcgccgcgccgagcgcgggcgcgggacggatctggtcgcgcttgaacaccaccacgtcggatacgccgtcgacgacgattcccaccgtcctgccggacacgttgagaatgatagtgaccgtcgtgccgccgtattgcgcgtcgcccaacccgaacttcccgcgcaaatcgatgacgggcacgatgttgccccgcagattgatgacgcccttgatgtaa

SEQ ID NO: 74 - DNA, 2382 bp, Burkholderia pseudomallei
atgacattcaatcagaagatcatttccgcattcgcggtcatcatcgcgggtgtgatcgggatgctcggcatggagatgtggtcgttgaacaacatcgaaagccgcgtgacggaactgcacgatcagcgcctcgcgacgctcaccgtgatgagcagcatcgaggacgatgcgctgaaggtgtcgctcggcatcgcgcaggcagccgactacatatgggtgaagaaggtcaaggcgatgagcgcaggcggggtggtcggcatcggcgaagaggcggtcgaccgcaaatcgttcgagtcgcacgtcgaaggcgcggagcgcgcgcttgcccgcacgatcgagaaactcgagcacaacaagatcaaccgcgaggggctgtccgccgacgaagctgcgaagattgaacgcatccgcgtgcagctcgcgcaactgcagccggatgcggtcaaggacgcgcgccgtatcctcgatatcgcggcgagtggcgaaagcgatccggacaggtatctggcggcttacctctccatgcggtggcagccgttttacgagaaggtttcggaactgtcgagggcttcgacggcggccacgagcaagagcatcacggacacgctggataccgttcgcgagcgccgcaggcacgtcgccgtcgtcagcctgttcgcgctcgtggtgctggccggcatcgtcgtctggcagatctggtacatcaagcgttcgctcggcggttcgatggaagcgctgtcgcggcacatgacatcgatcgcgcatgacgatctgagcggcgacatcagtgtggacgagcgcgacaagcagagcctgatgagctcgctggccgtcatgctcgagcgcttgcgcggagccaaggcgatggttgtcgaaaacgcacgcattcgcgtcgcgctcgactccggctcggccaacatcatggtcgtcgacgattcgcgcaaggtcgattttgcgagccactcgctgatgcgggcgtttgccgcgatgcagggcgcattgcgccgcgaggcgccgcaattcaattcgaatcagatcgtcggcgcgctgatcgacaccgtactcgagcgtcgcgtcgcggacgaactgctcggcgcgagcagccagcagcgctgcgaggtcaaggtcggcggccgcgactggctgctcgtatcgagtgtcatcgtcgacaacggcaaacggctcggttttgtgatcgaatggatcgaccgcacgaacgagttgcagatcgagcgcgaagtcgaagccgtgatcgccgcggttgccggcggagacttctcgtcgcaagtggcggaagcgggcaagacgggctttcatcagatgcttgcccagcggctgaaccgcctgacgaagaccgtggcggacgtggtgacggaagtggccacgacgttcggcaagctggctgacggcgacttgcgcacgcgcatggcgggcgcttaccaaggcgtgttcgccgaactgaaggagaactgcaatcgaagcatcgcgcagctcgaacgcatggcgatccgcatccgggacggcgcgcacacgatcaacggcgccgcgcgcgaaatcgcgtcgggcaatgcgaacctgtcgtcacgcacggagcaacaagcggcgagcctcgaacagacggccgcgagcatggaggaactgacaagcaccgtcaagcagaacgcggagaacgcgcagcaagcgaacacgctggcgctcaacgcatcgtcgatcgcgcagcggggcggccaggtggtcggccaggtggtcgagacgatggggcgcattacggcgagcgccaagaaggtcgccgacatcaccaccgtcatcgacggcatcgcgtttcagaccaacatccttgcgctgaatgccgcagtggaggcggcgcgcgcgggtgagcagggacgcggcttcgctgtcgtcgcgagcgaagtgcgcaatctcgcacagcgttcggcggcggcggccaaagagatcggcgcgctgatcggcgaatcggtcgtcgagaccgattccggtgcggcgctggttcagagcgccggcacgacgatggacgagatcgtgaatgcggtgtcgcgcgtcgtcggtatcgtcggggagatctcgagcgcctgcgtcgagcagagcagcggcatcgagcaggtgaatcaggcggtctcgcatatggacgagaacacgcagcagaatgccgcaatggtcgagcaagctgcggctgcggcggaaagccttcaggagcaggctcaactgctgcacggcacggtcgagatgttcaaggtcggcgaacggccggctatcgacgcgttgccggaggtccgagccgtggagccgccgcccgcggtgccagccgtgacccgcaggcgcgctggcgccgcatcggcggccgatgaatggaccgacttctga

SEQ ID NO: 75 - DNA, 822 bp, Burkholderia pseudomallei
atgaatagcgtggattttgaatacacgaacgcggatttccgccggattgcacggcatgtctatcagcgcgcggggatcgtgctgaccgaggcgaagcgctcgcttgcatacgggcgcctggtacgccgcttgcgtgtgctgggtattgggcgcttttcggactatctcgaccaactcgaggccgatcggtcgggtgacgaatggcagcatttcgtcaacgcgctgacgaccaatttgacatcattctttcgggagagctaccacttcgaccttctcgcgacgcagatgcgggcggcttcggggccgttccggatctggtgcgcggcggcaagcacgggcgaggaaccctactcgctcgcgatgaccgcgtgcgatgcgcgcgggacggcggcgaaccgcgtggagatcgtcgcttccgacatcgacacgaaagtgcttgccgcggcgcaagccggcatctacaagctgcatcagctcgacaagatgccgcttcgccgcaagcaggcctattttcagcgaggcagcggcagcaacggcggcacggtgcgcgtcaagcccgcgctgcgcgagatggtcacgttccggcagatcaatctgctgacggaatcgtggccgctggacggctcgttcgacgcggtcttctgccggaacgtcctgatttatttcgacgccccgacgcagcagaaggtgttgcgcaacatggcccgccacctgaagtcgcacggccgcctgtacgtcggccactccgagaacctcagggccctcgacgatctctacgcaccgtgcggccgcgcagtctatgagctcgtgaatgggcgttga

SEQ ID NO: 76 - DNA, 603 bp, Burkholderia pseudomallei
atgggcgttgacggtgtgggctatcacttctacttcgaccgcgaattcggccggcaggcggtacgcgtcaacccgggcggcttcgcgatcgcttcggcggacgtgatgctcgcgaccgtgctgggttcatgcgtatccgtttgcatgttcgattccgccgcgcgcgtcggcggcatgaatcatttcatgcttcccggaagcggcaacggcgcgcggcacgacagtctgtcgtccctttatggcgtgaacgcaatggagcggctcatcaacggcttgctcaggcgcggcgcgctcaaatgccggctgcgcgcgaagctgttcggtgggggctgcgtgatgccatccctgagcgacacgcgcatcggcgagcgcaatgccgcgttcgtgcgcgcctacctcgacgcggaaggtatccgttcggtcggcggggacatgctcggcacgcggccgcggcgcgtgtgctatttcccgttgaccggccgcgcgctgtgcaagcggctggtttgcggaagcgatgtcgccgatatcgcgacgagcgagcgggcatatgacggcgacctggcacggcggttgcccgtggccggcagcgtcgaactgttctag

SEQ ID NO: 77 - DNA, 1035 bp, Burkholderia pseudomallei
atgtcgatgcgaacgttgctggaacgcatcatcaatggttgcgacggcatgacctgcgttggcgcggcgcaagacgcgaacgcggcgcgggagatgatccgcgcgctcgatcccgacgtcgtcacgctcgacgtcgaaatgccgggcatggacggcttcgaattcctgcggcgcatgatgctcctgaagccgaccccgacgatcatggtgtcgggacgtacgacatccggctccgatgccgcgctgcgcgcactcgagcttggcgcagtcgacgtgatcgccaagccggtgttgaccgggccggccgatcttgccgactatgcgcgcgacatcgcggagctgatccgcggcgcggcggccgcacgcgtaaaaggcggtgtgctcgccgccgcgtcgcccgcacgaggcgagcgtgcgtgtattcaacggctgcgcggcgcgaagaaggtcggcatcgcggcaacgcgtctgagcagggtgatcgcgatcggtgcgtcgacgggcggcacggaggcgctcagggccgtgctgcaggacatgagcggcacgccgccgccgattctgatttgccagcacatgccggaggggttcacggcttcgttcgccgcccggctcgacacgatctgcggcattcgcgtcaaggaagcggagcagggcgagccgctgcgctacggctgtgcatatgtcgcgccgggccactcgcatctatccctggcggctaccggccgccactacgtctgccggctcgaggcgtcgccgcccgtcaatcggcaccggccgtcggtcgacgtgctgttcgattcggtcgcgcgcctcgcgggcaagcgcgcgctgggcgcgattctgaccggcatgggcaaggatggcgcggcggggctgctgcgcatgcgagcttcgggagcacggactttcgcgcaggacgagcaatcgtgcgtggtgttcggcatgccgaaagaggcaatcgcgatgggggcggtggatgaaattctgccgcttgcccgcatgggcgcgcggctcagggaagcgttgcaatga

SEQ ID NO: 78 - DNA, 1050 bp, Burkholderia pseudomallei
atgattcgcgtactggtgattgacgattccgcgacgatgcggatcttgctgaagaagcttatcgacaagaacgaacacatggaatgcgtgggcgtcgcgcccaatccggtggcggcccaggatctgcttcgggaagccaggccggatgtgattacgctcgacatcgaaatgccgaagatgaacggcctcgatttcctcgatcgcatcatgcggctgatgccggtcgcggtgatcatgatctccacgctgaccgaggccggttccgaatcggcgctgcgtgcgctcgaactgggcgcgatcgatttcatcgcgaagccgaagctcgatttcgccgaaggcgtgcaggcgtatgcggaagaaatctatcgcaagatcgaaacggccgggcgagccaaggtgaagaagctcacgcgcgacgtgccgcccgtgcgaatggatgccgaaccgcccgccaagccgttgctcgcggaggcgggcacggaaggccgcgtcgtggcggtgggcgcctcgacaggcggcaccgaggcggtgaaggaactgctgttgggtcttccggcggattgtccgccgctgctgatcgcacagcatatgcccgagccgttcatgcgttcgctcgcgaagcgtctggacctgctgtgcgcgatgcgcgtcaagatggcggaagacggcgagacgctgcgtcgcggttgtgcgtatatcgcgccgggacattcgaacttgaccgtcgaggcgacggcagccgggtatgcatgccggatcgtccggaatgccggcgaggcgcaggcgggctcgtccgtggacgagttgttccggtccgtggcggccgccgcgggtgcgcgcgccgtgggcgtcgtattgacgggcagtggctcggacggcgccgccggcgcgcgcgcgatgatggcggccggagcgttcaacattgctcaggatgcggaaacgagcgtggtctacagcatgccggacgcggcgatcgccgcttgcggcatcaacgaagtcctgccgctcgagaagatcgccggaaagctgatggaactcgaccgcgcatga

SEQ ID NO: 79 - DNA, 2091 bp, Burkholderia pseudomallei
atgaacaaactgacacacctgttcgccgacttgagacagtatcaatatgcgacgccgatcctgctgctggcggtgctgtcgatggtcatcctgccgctgccgcccgtcattctcgatctgctcttcacgttcaacatcgtcctcgcgatcgtcgtgatcctggtcagcgtgtcggtcaagcgcccgctcgaattctcggtcttcccgaccatcatcctcgccaccacgctgatgcgcctgacgctgaacgtggcgtccacgcgcgtcgtgctgctcaaaggccacgagggcaccaacgccgccggccacgtgatcgaagcattcggcaaggtcgtcatcggcggcaacttcgttgtcggcctggtcgtgttcgtgatcctgatgatcatcaacttcgtcgtggtcacgaagggggccgagcgtatctcggaagtgtcggcccgcttcacgctcgatgcgctgcccggcaagcagatggcgatcgatgccgatctcaacgccggcctgatcaatcaggagcaggcgcaagcgcggcgccgcgacgtcactaccgaggcggatttctacggcgcgatggacggcgcgtcgaaattcgtgcgcggcgatgcgatcgccggcattctgatcctgctcatcaacatgctcggcggcctggcgatcggtgtattcgctcacggccaggctttcggcgaggcattccagcgatatggcctgctcacgatcggcgacggcctcgtcgcccagattccgtcgctcctgctcgccgccgcgtcggccgtgatcgtcacgcgcgtgagcgattccggcgacttcgaggaacaggtcgggcgccagatgcttgcatcgccgaacgtgctctatagcggggcggcggtgatgatgatcctggcgatcatccccggcatgccgtggttgccgttcagcgcattcgcgggcgtgctcggctacgtcgcgtggcgcgtcgccaaacggccggcgcccgagcgcgaggaaacgcagatccacgcgctcgaaacggcgctgcgcgaatcgaacgtgcaggatctcgactgggacagcatcagccacgtgcatccgctcggcatccgggtcggatacaggctcgtcgatctggtcgatcccgcgcacggcgcgccgctgcgacggcgtgtcgacgggatgcgacggacgatgagcgaatcgatgggcattctcgtgccgccgattacggtccgggacgacttgtccctgccatcaagtcactacgcgatccaccttcacggcgtcgagatcgaccgcgctgaactgcatccggatcatctgatggcgataccttcgccgacgacgtacggatcagtcgacggcattcccgcagcggaccccgcctacggcatccccgtgacgtggatccggccgcaggacaaagcacacgcgatcggcctcggctatcaggtggtcgacggcgcatctgtcgtcgcgacgcacgtcgacaagctgctgcgcgaccatctaggcgaactgctgacccacgatgatgtcggatcgctgctgcagcggctcggtcaggtcgcgccgcgcctgctcgaagcgctcgagaaagcgatgccgcataacctgatccgcaaagtgtgccgcgtgctgctgacggaccgcgtgccgctcaggaacatcgtggcgatcgcgacggcgctgctggagaatgctgaagcgacccaggacccgatcctgctcggtgcggaagtgcgctgcgcgctgcggcgcgagatcgtcgcgggcattgtcggccatcggcgcagcgtgcccgtcttcaatctggccggcgagctggagggcattctgctcgccgcgttcaatcaagcccagcagagcgccaaggccccgctcgacaacttcgcgatcgcgcccgacctgctgacgaacctgcaaagcagcctgcccggcgtccaggagcgcatgcgcacgctcgggcacgcgccgatcctgttcgtgccgccgcaactgcggccgctcctcgcgcgctacgcgcgcgtgttcgcgcctgggctgcacgtgctgtcgtacaacgagatcccggacgaacgtcagatcgagatcgccggcacgctcgaatga

SEQ ID NO: 80 - DNA, 1008 bp, Burkholderia pseudomallei
atgctcgtgttgctcgcgccctcgtggctcgccgagtttcgcgcgctgtttctgctcgagctcgtcgacgtcaccggcgacggcgcattggccaacgcgtggtcggtcgcgctgccggtatcgatcgcgctgttcctcaagatgatcgcgccattcttcgtcgtgccactttgcgtcgtgctcggttcgctgctgcccggcggctggatactcagcggcgcgcagttgaaaccgaagttctcgcggctcaatccgatgacgaatctcggccgcctcgtctcggcgcgccactacggcaccttcgcgctatcgctgctgaaagcgctcgccctcatcgcaatgcttgcgtatctcagcgcgacgacgctcgccgcatttctgcggctgcagcacttgccgctgcaggaagccttgacgcgcggcttcggcctcacgctcgacggcgcgctcgcgctcgccgccgtgatgctcgtcttcgcgcttgccgacgtgccgttccaacgcttcgttttcctgcgcggacagcggatgacgaaacaggaagtcaaggacgagcacaagaacgcggaagggcgcccggaagtgcgctcgcgcatccgccagcttcagcggcaagccgctcagcgcgtactgtcgaagaccgtgccgagcgcggatgtggtggtcgtcaatccaacccattacgcggtcgcgctcaaatacgacacgaaccgagccgaagcgccgttcgtgatcgcgaagggcgtcgacgacatggcgctcgcgatccggcggatcgcccaagaacacggtgtcgaggttctgagcctgccgccgctcgcacgcgcgatctacgacacgagccaggtcaaccagcaaattcccgccgcgctctatcgcgcggtcgcccaggtgctgacctacgtgctgcaactgaaagcgttcaggcaggggcgccgcggcctgcgccccgaacgcccgaccgatgttgcgattccggattttctcgacagaacacgaccctcatga

SEQ ID NO: 81 - DNA, 777 bp, Burkholderia pseudomallei
atggacgccatcgtcgccgccgctgccgagtggctcgcacgggcatggtggccgttctgccggattctcgccgcgttgagcgcagcgccgttcatcggcgaaagcaccgtacctgcgcggatgcgcgtgacgatcgcactgttcctcgcgctgatcgcgctgcccgccgtcgaccccgtcgccattccggtcttctccgcccgcggcgcgctcgccgccgccgagcagatcctgatcggcttgctgttcggcatgctctttcacttcgtgcaggccgtcttcatgatgctcggctacctcgtcgcctcgcaaatgggcctcgcgatggcagtgatgaacgacccgctcaacggcgcgacgtcggatgtcgtgtccggcttcatgctcgtggcaagcatcctgctgttcttcgcgatcgacgcgcacctcgtcgtcgttcaggtgctctatacgagttttcgcgtctggcccgtcggcacgccgatcgatctgccttcgctcaagccgctcgcctaccaggccgcctggctcttctcgaccacgatgctcctcgcggcgccgctcgtgttcgccacggtcgtcttgcagatcggcaagagctatctgtcccgcgcgacgcccagcatcaatctgttttccctcggcttcgcgatcatgaccgtattcggactgctcatgctcggcgccatgctcaagggcctgcccgcgcattacgccagcatgacgcggcacgttctcgacgtgctcgagcatcatctgggagccgcgcgtggctaa

SEQ ID NO: 82 - DNA, 273 bp, Burkholderia pseudomallei
atgcttacctccgatctcgccgtcgaactcgtcggcaatgcgatccggctcgtcactctgatggtcgccgtcatcatcgcccccgggctgctggttgggctggtcgtcgcgttgtttcaggccgcgacccagatcaacgagcagacgctcagcttcctgccgcgcctgctggcgacgctggcgatactcgtgctggccgggcactggctcgtacagacactgatggattactgcgtcgcaaccttcacgcgcgcggcacaactggtcggctga

SEQ ID NO: 83 - DNA, 768 bp, Burkholderia pseudomallei
atgacgcggctgcggttccgggccgcgcggcttgcgacggcgctgaccgccgcggccgccctgagcgcgatcaccgccacgcccgcgctggccgagccgctcaagctgccgctcgaatcgctcggcttgcgtggcgattacagcgtcaagacgcaaatcctgctgttcatgacgctgctcgggctgttgccggccctcgcgatcgtgatgacgagctttcttcgcttcgccatcgtgctcgcgctgctccggcaggcgctcgggctgcagcaggggctgccggctcgcgtcatcacgggcgtcgcgctgctcctgaccctgctcgtcatgcgccccgtcgggctcgacgtctggcgccatgcgatcgagccctatgatcgcgacgagatcacgttccgcgaagcgatcgcgcgtgcggaacagccggtctcgcgcttcatgctcgcgcagaccagcaaggccagtctcgaacagatttcgcggctgtccggcgagcgcgatgtcgcgaagccggaggatcacgcattcgtcgtgaaggccgcggcgttcatgatgagcgagctcaagacggcgtttcagatcggcgcgatgttgttcctgccgtttctgatcatcgatctggtcgttgcttcgacgctgatggcgatgggcatgatgatgctgtcgcccctcgtcatctcgctgccgctcaagctgctcctgttcgttctcgtggacggctggaccttgaccgtcaacaccctgaccaccagtatccaggcttactga

SEQ ID NO: 84 - DNA, 348 bp, Burkholderia pseudomallei
atggactcgaacgagccgaacgacctcgacgcactgctggacgatctgccggcggatgcgccggcggataccgcaacggcggcgcacgcaccgcgccgcgacccgcgtcaactgctggccaagatcccggtccagctcacgcttgaagtgggaacggccaccgtctccctcgcggaactgatgaacatcgatgaaggcactgtgctcgaactcgacaggctggccggcgagccgctcctcgtcaaggtcaacggcacgaccgtcggccacgcggaagtcgtcgtgtccggcgagaactacgggctcaagatcatcgatctcgagaacctcggagattttgcgccatga

SEQ ID NO: 85 - DNA, 870 bp, Burkholderia pseudomallei
atgctgggccgaccgacgcacttgctggacgggttcggcgcgacactcaggcaatacctcgaccagttcttcacgacgagcctcggccgcaggcacggcgtcgccgtggcggtgcgaaacgtctcgttcacgtcgcatgtgcccgaactggaagcgcggccgtggcggcgcgaaacagtcgaccgagcggcgatcggcgtgcacatcgagcgcgcgctgctgctgctgcttctggagctgcgctacggcccggcgatctcacccgtcatgccccccgccgctccggccgatgccggcgacacgccgccggcaagcgacggcgaaaccgcctccgccccaccgacgccagccgaaacggcggcgacgcacgcaccgccgattccggaaaccaccaccgaacgccggctggccgagcgcctcgcggcggaactcgcccgcacggtcgctcgcgcgattcctttcgacgcgcccgccggcgagccgccggcgccgtcgagcggcgcccatctgttcgcgatctgctcgatcgaaggcgctgcgtcccggacagtcggcgaaatatgcttcgcgctcgatccagtctggcagcagcgtctgttcgaccacctgaaatcgtcgatgagccgcgcgcggagcgtgaagcgggaacgtccgtcgccgtcgacccagttgcgcctcaagctgacggcacagctcgtggagatgagcgttcctttcggcgacgtgctccgccttcggcccggcaccgtgctgcctgtccgcctgcatacgcccgcgcgggtaatgacccacgacgcgcagttgttcaccgcgtcgatcgccgagcacaacggcaagctttgtctgacttcctttgtctacgtggagtaa

SEQ ID NO: 86 - DNA, 333 bp, Burkholderia pseudomallei
atggaatcgattcaacaggtgtcgtcgcaattgcgcggtcggatggcggaactcgccggccaggccgcgtctttgagcggcgcgattgtcgggcagtccggcggctccggcgaggtgaatttcacgcacatgctgggctccatgctcaaggaagtggacgaccggcaactcgacgcggcggaaaaagccgtggccgtggacagcggccgaagcgacgacgtcatcggcgcggtgctctccagccaggaggcatcgttgtcgttctcgatgctgacccaggtgcgcaacaagctgagcacggcgctcgatgatctgctgaagatgcagatctga

SEQ ID NO: 87 - DNA, 1596 bp, Burkholderia pseudomallei
atgccttcgccggccgcgctgtcgcgactgcttccgctcgtcgtgctcgcggtcgcgttgactgccgcggcaacgctctacatgcgctacgaccaggcgaattacaagccgctgttcggtgcgcgcgagccggtgcaactcgatgacgtcgtgtcgacgctcgacgcccagcgcatcccgtaccgcatccatccggataccgggcaggtgctcgtcgcccgctcggacgtcgcgcgcgcgaggatgaccctcgccgcgcgaggcgtcaccgcgaaggccgcgccggggctcgagcagatcgatcaggacgatccgctcggcacgagccagttcgtgcaggacgtgcgattccgccggggcctcgagagcgaattggcgcaaagcatcgcgacgctcgaaccggtcgagcgcgcccgcgtgcatttatcgattgcgaagtcatcgtcgttcgtgatgatggacggcgacaagagcgccgcctccgtcgtgctgacgctcaagccgggaaggcgtcttgcgaaggagcagatcgccgcggtcatcaatctcgtcgccggcagtgtgccgaatctggcgccggcaaaggtttcggtcgtcgatcagcatggcgcgtttctgtccgcgcgcgtcgacctggacggtgatctgacgggcacggacgagatcgcggcgcggtttcgcgaagacgtgacacgcaatgtcgaggatctgttggcgccgtcgctcggcctcgggaatttccgcgtcagcgtgacggcgctcgtcaacaacgaccgtgtcgaggaaacgcgcgagcagttcggcgaagcgccgaaggtgatgaacgaggcgacccgcaacgagcagaaccgcgatcgcgtcgcgctcggcgtaccgggctccttgagcaaccgtccggtcgacgtgcccgcgtccgagccgcaggccgacggcggcacgcaacgcgccgcgacgacgcgccagttcgcatacgaccgcaacatcacgcagatcaagcgtgggcgcgggcagctcgagaagctcagcgtcgccgtcgttctcaacaacgccgtgtcgcccgccaagggcaagccctggtcgccggagcaattgcggcgaatcgacgagatcctgcgcagcggactcggcatagacaagcagcgcggcgatcaactgatcgtgtcgtcgctcgatttcgcggcgccggccgctgccgagccgtggtggaaggagcgcgacaccctcgagcagggcggctggtatctgacctacgcgctggcggccctgttcggctacctggcgctcgctcgccccatgctgcgcatgctccagcaatgggtgcagcaccgttatggcgcgccggccgccgctgcttcggcgtccaggctgccggcccccgagggcggccacgcgcaagcggcactgccggctgcggacgacgcgcggccgcccttggcggcgatgttgcctctcctcgatgacgtggagctgccgcctgccgattcgggcgtggaggtgctgatcgaccacttgcgttcgctctccgagaaggagcccgagcgcgtcgctgaagtcgtcaaacaatggatacggaatgatggaaacgccagtagctga

SEQ ID NO: 88 - DNA, 1017 bp, Burkholderia pseudomallei
atgatggaaacgccagtagctgatagcaatgcccagggctcgatcgagcaggcggcgatcatcctgctgagtatcggcgaggagcgcgcggcggacgtgctgcgctgtctgtcgcgtccggaactgctgagaatcacgcagacgatgtccagcatggcgggcgtcaaggtggacgtcgtgaaggacgcgttgcagcgcttcttcgatcagtatcgcgaacaaagcggcattcacggcgcgtcgcgcgtgtacctgcagcgcgcgctggatctggcgctcgggcgcgacatcgcgaacacagtgctgaacagcgtatacggcgacagcatccgtccgaagatggcccggcttcaatgggtgtcgccgcgctggctcgccgaccatatcgttcatgagcacgtgcgcatgcaggccgtgtttctcgcattcttgccgcccgcccaggcgagtcaggtcatcgagatgctgccgccgcacacgcgcgacacgatcctcatcaatgtcgcgaagctcgacgaagtggatcacacgctgctcgtggagctcgaggagctgatcgagctgtgtctgaaggacctggatctgcagggcacgaccgtggagggtgtgcgacaggccgccgagatcatcaaccgcttgccgggcgaccgcatgcagatgatcgagttgctgcgcgcgcacgatcctggcgtcgtcaccgagatcgagaccagcatgtatcacttcgacatgctggcgcgccagagcgacaacgcgatcggcaggatcgtcgaggtcgcgccggtcgaacagtgggcgatcgcgctcaagggcgccgatccggccgtgctcgcggcgctgcaacgggtgatgccacggcgccaggtgcaggcgttcgacgacacgattcgccgcacggggccgctgccgtccacgcgaatcgagcaggtgcggcgagacatcatggcgcgcgtcaaggcgctggccgacgagggcgagatcgagttgcgcctcgtcgaggaggacgtcgtctcgtga

SEQ ID NO: 89 - DNA, 588 bp, Burkholderia pseudomallei
atggaagacggttatcgcgcgggattcgagcatggggaggccgaggggctcaagcagggtcgcgatgccgggtttgccgccggcgcgcagcaggcgcgcgatgcgctgcgcggcgaatacgcgggcatcgcgaaaacgctcgatgcgatgcgcgacgcgttcgcgcgcgttcaggccgagtacctcgcggcccgccgcaccgaactggtcgacgtggtcgcgaaggtggccaagcaggtgatccgctgcgagctgacgttgcagccgagccagatggcggcgctgatcgaggaaacgctggaaacgatgccggcatcgggcgaggctcccgaggttcacctgagccccgacgatcatgcgcgtctggccggcctgttgccggagcgcgcgcggcattggcggctcgttcccgacgcgcgtctcgaagcgggcgaatgtcgcgtgttgctgggcggcaacgaagccgatgccggttgtcagcatcgtctcgacgcctgcgtcgagcgcatcggcgaacaattgccgggcgctccctctgacgttgcggcgggcgcgacgatcagcgacgtgcaggaggaggcatcgtga

SEQ ID NO: 90 - DNA, 1254 bp, Burkholderia pseudomallei
atgacgggccgcctgaccggcgtgacggggctgctgctcaaagcgtccggttgccggctcgaaaccggcgagcgttgcgtgatcgaatcggcggacggagacaggatcgatgcgcaggtggtgggcttcaatcgcgaggagacctttctgatgccgttcaagaagccggtcggtctcgtcgcgggcgcgcaggtgctgtcgtgcgagcggcagggcttgctgcggatcggtcgctcgtggctcgggcggatcgtcaacggcctcggcgaggccatcgacggccgcggctcgctcggcggcgatgcggtgctcagggcgcatcccgcgccggtgaatccgctgcgcaagcgcccggtgcgcgacgtgctggacgtcggcgtgcgtgcgatcaacggcctgctgacgctcggccgagggcaacgtatcggtctgatggcgggcagcggcgtcggcaaaagcatgttgctcggcatgattacgcgcgcgacgcaggccgatgtcgtcgtcgtagggctgatcggcgagcgcggacgcgaagtgcgcgaattcgtcgaccacgcgctcggcgaagccggcctcgcgaaatctgtgctggtggccgcgccggccgatgagtcgccgctgatgcgcctcaaggcggcggagctctgccatgcgatcgccgctcattttcgcgatacgggcgcgaatgtcctgctgctcgtggattcgctgacgcgctatgcgatggcgcagcgcgagatcgcgctgtcggtcggcgagccgccggccacgcgcggctatccgccgtcggtgttcggcatgctgccgcagcttgtcgagacggcgggcaatggcgagggcgcgggcagcatgagcgcgatctacacggttctcgccgaaggagacgaccagaacgatcccgtcgtcgataccgcgcgcgcgattctcgacgggcacatcgtgctcacgcgtgagctggccgagcgcgggcactacccggcgatcgacgtgccgcgttcggtcagccggtgcatgagtcaggttgccggcgccgagcacggggcgctcgcgcgcaaggtcaaggaaatgatcggccgccacgagcagattcgcgatctgattgcgttgggcggatacgtgcagggcgctgatccggcgacggacgaggcggtggcgaaggcggcgcgcatcgacaccttcctgcgccaggacatgctcgacgtcgcgcgtcacgacgccacgctggcggcattgaaggagctgatcgcatga

SEQ ID NO: 91 - DNA, 438 bp, Burkholderia pseudomallei
atgaacgaacaacgcaaagtcacgcgcatcgcgggctggctcgccgcgcgccaagccgaggtcgacaggctcgccgcggacaccgcgcgccaggaagccgtctgccagcgctaccggcgctacctcggcgcgatgcaacggctgtacgacggatccgccgcggcgaccgccgcggccctgccgatgcttgcgcagaaccacgccggttatcgccagacgctgctcgacaccctggcgcaactccgggccgcgttgaaggtgcaagaggacaacctggaagtcagccgcgcgctgctggccgcgaagacgctgcggtgcgaagtgtggcgtcgcgagctcgatcgcaggcggcgcgcgctcgagctcgcgcggcggcgcagcgagcagcgtgtgcaggacgaactggcggtgcaggcgtggcggcggacgccggcatga

SEQ ID NO: 92 - DNA, 1326 bp, Burkholderia pseudomallei
atgttgagagtgacggatccgcaggcaatggcgaagactttcgccgaggcctacatggaaagcgcgaggtggcagctcgagcggcaagcgaatgacgtgaagaggaacaaggatgcgctgtcgggtctgggcagcttgctgaccacgttccgggacaagcagcgcgagcttgtcgcgggcggcagcatgatccagaacacggtgacgcaatcgaacgatgcgttcgcgtcggtagcgacgacgtccggggcgaagccgggcagctaccagttccatgtcgaacaactcgcgacggcgcatcagctgacgatcgacgtgccgtccggcattcccgccgcggacgccggctcggtgaagatcaagctggccgacggatcggaattcaccgtcgacctcacggcggccaagggtgaggatggcaatgtgtccgccgaagaactggctcgcgtaatcaactcgagttcgggggcgagcggcaacgtccatgccgcggtgatcggcggccagctcatgctgacgtcggccaatacgggggccgacagcaagatttccttcgagatcgattccacgtcgccgcttgccggcgcgtttgacggcgccgtgacgatctccgaggcgcagaacgcgaaggtcaagatcggcgacccgacgaacggaaagacgatcgaactgcagggcaacacgcttgattcgatcgaagggctgagcatcacgttcagacaggtcggatcgggaacgattacggttgcttccgacaaggaaaagacaaccgagagtgtcaaggcgtttatcgatgcgtacaacgaactgatcgacaagctcgccgagctaacaagagtcggcgatgcggcgaaaggtgatccgggcgggccgttctcgtccgacgcgggggtgcgcgcactcgtcagccagctcagatcggagttgaacaagagcgtggatggcgtgaagttgtacgagctcggtatcgatacggacaagaagggcaagctggtcctggatgaaaaacgtttcgagcaggcgttcaaagcgtcgccggatcgcttcgacaagctgttcggcgatgcgcagaccgggctcgtcggcaatctcggcaaggcgctcgacggctggctgaacgaaaagaacggccaactgaagacacgcaacgaatcggtcgcgcaggcggaaaagcagcttctcgccaaacagcaagagtacgaccgccgctaccaggacgtgttcgaacgctatacgcaggaattcacgcggcttcaggtgctccagctgcaaatggacaacacgctcgaccagatcaagaacctgttctcgacgagcaaggattga

SEQ ID NO: 93 - DNA, 171 bp, Burkholderia pseudomallei
atgacggcgggcagcgaggtggtgacgaacctggcggcgctttacgactactgcgtgcgccgcctgaacgttgccggcatcgaactgaacgtcgagccgctcgacgaagcggcgcatctgatcggggtgcttcgcgaagggtggcagggtgcgcaagatggcggactttga

SEQ ID NO: 94 - DNA, 330 bp, Burkholderia pseudomallei
atggcggactttgatgttgatcgggtgggcgacttgtccgctgcgttgacgacgcgcgcgcgccggctcgccgacgccgcgatgcagcgcgactgggcgtcggtgacggcgctggatgcggagctgaacgcgatcgtgcgccgctgcgccgcggacggcgagccgacgcgggacatgcatgccgcgttgcaggccgtcgcgcgcgtgtacgaacgggccgccgcgacgatcggcgacgcgcacggcgaactggcgagcaggcttgtcgagctggccgagaaccggcacgcatggatcgcctacgcgacgatcgccgagggcgggcaatga

SEQ ID NO: 95 - DNA, 1152 bp, Burkholderia pseudomallei
atgaacgcgatcaccgcgatgacgggaccggcgaccgatgcgagcgggcgcgccggtgcggcttgcaccgtgctgctcggcgctggcacggtgcgtggacggccggccggcgcatcggacgacgaggcccggacgcagggcgccgaatcgccgccggagacggatcgtttggaggatgtttcgcaatccggcgcattcgccggggtgatcgcggcgctcgaggttcaagcgcccgttcgggatgtgcaggccacggacgattcggcgagagacagcgaggatgccgcgcgtgagcgcgttgcggcggccgcggcagatgcagtgacggtgggtgggaccgtccgtgcgttgcgagatgggctcggcggactcgcgtttggacagagtccgatgatcgaggcgggtaaggcgctgcgcgtcccgccggcgcgcgatggcgtacggctgcccaatgaagacccgctgcgcgacggcgaacggctccgcgacgagactggcggtgcgcgcgctggcatgcgcgattcggtgtcggccgccggcgcggcatcggcggcccgtgtcgggcagccggccgcgggcgcgccgcgcgatcagcaagccctgtcgcaggtgcccggtcaaccggccgccgacaagccggcggccggtgcgcctgggccgcaggacggtgcgatcggtgcgatcggtgcgcccgttgacgtaccgcgcgcggccgctcgtgcgtattcgccgcagtccgcgcccgtgcgcctgggagccgagctgacgcgcgtactgggcgagcgcatcgatgtgcagcttcaacgcggggtcgagcgcgcggtgatccggctggaccctcaatcgatgggcacgcttcatatcgcgattcgtcatgagggaggaaccgtccagatccagatgatcgcttcgcatgaagaggtcgcgcgccaattgcagacgatgagcgaggcgctgcgccaggatctgtcgtccaggcatcacggcgatgtcaccgtggtcgtgaggcatggccagggcatggaacaaggcgcgcgccatggcgatggcggcgcgcaccgcgaagagaagcggcccggcccggcgcttgccgaggctgacgtgccggacggcgacgaagggtttcacctcggcggacgagacgtgatttaa

SEQ ID NO: 96 - DNA, 465 bp, Burkholderia pseudomallei
atgaaaaagaagaccgttgtgtcgatgatcgtcggcgcgacgttactggttggcggcgccatcggcggtacctgggcgtttctgagccgcaacgcgagccatcgtcccgcgccggtcgcggcgcaggacaagtacaagtacgtcggggaaagccagattgtcgtgatgttgcgacagcaggacggccagaacgccacgtcgatcgatgcgcaggcgcactacgcgatgctcgacctcgtgctgaaaaccacgcccgaaaaggaagccattgtccgcgatcacatgttgctgctgcgcagcctgacggtcgacgcggtgtcgcagtacacgctcgagcagctcaggcagatgaacgtcgcggagctggcgaacgtgctcgaccgcgcgttccggcagtcgtacaagacgcgggccgtcgcgatgccgttcgagagcgtgatgatcggcaagcttctcgccgaatga

SEQ ID NO: 97 - DNA, 711 bp, Burkholderia pseudomallei
atgacgacactggacgattgcgccgccgcgcgcactgcttatgcgcgcgccggagcgggcgccgagcagagatggctggtggagtacgcaccgatcgtcaagcgcgccgcccgccagttgaagtcgcaggcagtcggcatgctgacgctcgatgacatcgagcagattggcctgatggggctgctcgaggcgctgcgccgctatggtatgccagatgcggccttcccatgctacgcggcgactcgcgtgcgcggcgcgattctcgacgagttgcgccgtcacgactggcggccgcgttcggtgcgcctggaggcgcatcgcacgcgcgactgcgaacgcgagctgtgccgctcgctcggacgcgagccgacggaagccgaggtcagcgatgcgctcggcatcagcacgacggaataccgcgaacgcgtgatggcgaacaatgcggaggccatggcgagcttcgacagcgtgctcgacgacgacgcgttgacccacgaagaaagccccgaaagggagatcgtccggcgtcgctgcctcgagcaggccctcgctcgcctcaacgagcgcgagcagcgcgtgatccagctgtactacgaattcgatttgagcctgcgcgagatcgcgtccgtcctcggcttgaccgaggcgcgggtttgccagatcaacaaagcggccctcaagaagatgcgagaggtgttgagcgatgcatga

SEQ ID NO: 98 - DNA, 285 bp, Burkholderia pseudomallei
atgagattgttcagcacgaaatatggcgtgccggcgcaacgaagcaacggcgcgagccgtgcttcgacgacaacgcacgaaggcggcgcgcgcggcgcggcggcgggcgatgcgggtttggaggtcgagctgttgcggccggcccgggaggtgctgcgcgagctgcccgatttcgacgcggcgcgcgtggcggcgttgcgcgccgcattgcaggaagggcggctgccgttcgacgccggcaagctggccggactgatccagcgttttcacggagcgaaggaatga

SEQ ID NO: 99 - DNA, 426 bp, Burkholderia pseudomallei
atgacgcgcaaggaagcattctcgcgaatgctcgccgggctcgaggcggaccggagcagatgcgaggagttgaggggactgctcgagcagcagttcgatgcggcgctgcgtcgcgacggttcgttgctaatggcggcgggcagcgccattcgggcgctggtgcaagcgctcggcacgagccacgaggcgcgccggcaatgcgcgcgcgtgctcgccgcgccgaagcggcccgcgtcgatgagcgaactgtgcgcattgctgcggggcgcggcccgcgatgcgctgacgctcgaatggcagtctttcgatgcgctgatgtgcgagtgccggcgactgaatcagcgcaacgccacggtgctgatgtctcagttcgagatcgtgcagcgtgtgcttcacggcgagagcgatacctatgttcatccctga

SEQ ID NO: 100 - DNA, 795 bp, Burkholderia pseudomallei
atgttcatccctgaattcgatggccgttcttccgattgggcgcgcgcggcgtcagccggcccgggcgtcgcctcatggagcgcggcgcgaggcgagtttcgcgcggaattctcacgccttcagcgggatatcgccgcgacgatcgcgcaaggtttcgacacatccggcgacggcggccgcgttcgcattcagggttccgcgccgtttgccggggcgaacgcgatcgtacgtttgctggacgggggatcggcgcatgcggcgccggagaaggcggcatttatcgagcgcatcctgccgtacgcgaacgaggccggaaaggccttgtccgtttccggcgaactggtggcggcgcacgcggcgctcgagtcgggctgggggcggcgaccgttgacgacgggcgaaggcgcgaatacccacaacctgttcggcatcaaggccggcgcgaattggacgggccacgtcgcggacgtgctgaccaccgagtacgtcggcggcgagcccatcaagacggtggagcggtttcgcgcgtacccggattaccggtcggccttcgcggattacgcggcgctgttgcgcggcagttcgcgttttcgcgaggttatcgggacgggcgacgacgcggccgcgttcgccgccgcgctcgctcgcggcggctatgcgaccgatccggcttattcgtcgaagctgcgccggatcgcggccgacgtggcggcgtcgctcgccgccgccggcggtatcagcgagacgcgtccggcgctggctgggcaatcggctcggccgtccccggaccggtga

SEQ ID NO: 101 - DNA, 468 bp, Burkholderia pseudomallei
atgcagcgaacggcggctcgttcggccgatcgcctcgtcggcgcgggtcacggtgtcggtcagcgaagtcagtatgcgacgcgccgtgccgagatcgtccgccgcgatgggctcgcgggcatcgaggtcgcgcgcggcaacgggaatcgttgcggtgacctgggcccgggccgtgagcttcgtcacgcggccggatgcggggcagcgcgccgacagggcgagcctgccgagctgacgcggttcgccgacactgacttcgaacggcgtatcgcagacgggcgcgggcggcttgagcggcgaaacgacgaccgcgatatcggcgtcgcgcagtccggcgcgcttcgcgagttccgcgaggcgggtcagcgcgatttgccgagcctgctcgcgaagctcggggcgcggcgcggcggatgcggccggcgcggatacggccgccgtggacacggccgacgcggacgtggctga

SEQ ID NO: 102 - DNA, 375 bp, Burkholderia pseudomallei
atggcatttgatctggcgagcgggctgggccttcatcccaccgcgttgaagctgcgggcggaacgaacgaaggtcatcgcgtcgaatttggcgaatctcgatacgcccggctatcgggcgcgggatctggatttcaagcgcagcctggccgaggccggcggcgacggcgcgcacgcgcgtcccggcgacgcgctgacgggcggcgtgacgctcggctatcgcgtgccgtatcaggcggcacaggatggcaatacggttgaactgggcatcgagcaggcggcgttcgcgcagaacgcggtcgactttcagatgagtctcacgttcctgaacacgaagatcaagggcctgcaaaccgcgattacgggcaatgcgtaa

SEQ ID NO: 103 - DNA, 417 bp, Burkholderia pseudomallei
atgagcttcaaggacatcgcacagatagccggttccgccatgtcggcgcagacggtacggctcaatacgatcgccagtaatctggccaatgcggattccgcgtccggcaccgaggacgcgacctaccgcgcacgcaagccggtcttcgcgtcgatctttcagctcgacgcgcagggcgagccgcgaggcgccgccgtgcaggtgctcgacgtcgtgaagagcaccgagccgctgcgcaaggttcaccaaccggggcatccgcttgccgacgccgagggcaacgtgctgtatccgaatgtgaatccggttgaggaaatgacggacatgctgtcggcgtcgcgtgcgtattcgacgaacgtcgaagtgctcgggcgcgtcaacggcatgcagctcgaactgctcaagctcggtcagtga

SEQ ID NO: 104 - DNA, 696 bp, Burkholderia pseudomallei
atgtcgaatacgatccagccccgcgcgggcgatatgcagcaaccaggcgccggacagcacacgccgtcgccatacgccaacggcgcgaattcgccgacggatctgttcgcgaagctgctcgctgcgcaaatccagaaccagaatccgctcgaaccgatggacgcgtcccagttcgtcacgcaaatcatgatggggcagcagaccgaggcgctcggcgtcgtcgcgaacctcacgcagaacagcgcggcgctgatggaaagcatgctcgtcgtttcgctcgggtcgcaggtgggcacgcaggtgatggtgcgaaccgattcgctcgagctgaaagacgacacgcgcgacgcccgaatcgtgctcgataacgccgaatcggacgtgaccgtcgtgctgaccgattcagccggcaattcgcatcggatcgcgcttggcgcgcaagccaagggcgatgtgaatttcacgatcgatccggaggagctcggtttgcccgccggccagtacaagattcgcgtcgagaccgccagcggcgcgaaagcgggcgtcgagatcgaagggacgctcgagaacgtccgcctcgcggcggacggcaaggtgattcttcatgtcgccggcgtcggcgatgtggagacgtcgtcaatcagcagctttctgggtcgtcgacccgccaacgagacgcgcaaggaaaccgtatga

SEQ ID NO: 105 - DNA, 1188 bp, Burkholderia pseudomallei
atgagcttcaatatcgccctatcgggcatcaacgccatcaacagccaactgaacacgatcagccacaacatcgcgaacgccaacacctacggcttcaaggccgggcgcgcgaacttcgcgacaatggtcgcgggcacgcaggccaacggcacgtacatcggatcgatcacgcagagcgtcggcacgcgcggcggctttctgccgaccggccgcgcgctggacggcgcgatcgacggcaacggcttcttcgtgacgaaggatagcgacggctcgatgctcttcacgcgcttcggctacttccagcgcgacgcggacggctacatcgtcgacggcttcggcaggcgcgcgcagggttacggcgcgagcggcgcctatggcgacattcgcgtgccgaccgacacggagccggcgaaggcgagcgacagcctcgaatatgtgggcaatctgtccgccgactgggcggcgccgaagaacccgaatttcgataaggacgacgacacttccttcaatcattcggtcgcttcgaccgtctacgactcgctcggtcgtcagcacatcgtgacgcagtacttcgtgaaggggcagccgccgtcgaccgacgtcatcgcctattacgcgatggacggcgagatcgtcggcggcaatacgccggtccagacagtgttgcagttcgacacgaacggccaactgaccgcgccgaactcgccggtggatctcgatctcggtacgcccgccggcgcgtccgcgctcgcgatcaaggtcaactacgccggcacgacgcaagtcgccggcgagacgacgacgacggtcaatcgcgacaatggctatgcagcaggcgtgccgggagaggtgatgctcgacgaggacggcggcgtcgtcgtgcagtacagcaacggcaagcagcgcaaggtcggctcgctcgcgctcgcgaccttcgcgaaccaggatggcctgagcgccgtgggcgacaccgcctggcgcgcgtcggccgcgtccggcaatccgttgatcggctcggccggctccggcgcgctcggcaaggtggtggccggttcgctcgagctgtcgaacgcggacgtcacgcaggagctcgtcgacatgatgagcgcgcagcgcaactaccaggccaactcgaaggtgctgtcgaccgagaaccagatgatgcaggcgttgatgcaggcgctgtaa

SEQ ID NO: 106 - DNA, 726 bp, Burkholderia pseudomallei
atggacgcactgatctataccgtcatgagcggcgcggaccgcaccctgcatgggatcgcggtgcacgcgaacaacatggcgaacgcgcagaccgacggctttctggccgaactcgaaatggctgaatcgcaaccggtgcgcggctacggctatgacagccgtcacctgctgaatccccgggcgaacgcgctgaacggcgaggcgggcgtggtcgtggataccggccgcgagctggacgctgcgatcaatggcggcggctatttcacggtcgatgcgcagggcagcgaggcgtacacgcgcgcgggcaacttcgaaatcgacgccgacggcgcgcttaccgtcaacggccggccggtgatcggcgaaggcgggccgatcgtgctgccggaattcaccagcctgcgcatcggcgaagacggcacgatttccatcacgtcgccgggcgacaccgaggtgcaggcggtcgagcgcctcgcgctcgtcgacgcacccgcatcggcgctcgtgaagaacgaggcggggctgctcgtcgcgcgcgacggccagccgttgccggcgtcgaacgaggtcaaggtgctgggcgggcatctcgaacgcagcaacgtgtcgcccgtcgaggaaatgatcgcgacgatgtcgctcaatcgcgcgttcgaggtgcagatgcggatgttcaatgcggcgggcgagatggccgacgcgggcaaccggttgatccggggctga

SEQ ID NO: 107 - DNA, 789 bp, Burkholderia pseudomallei
atgaatcaagcaatgtggatcagcaagacggggcttcaggctcaggacgcgaagctgcaggcagtcgccaataacctggcgaacgtgaacacggtcggcttcaagcgcgaccgggtggtgttcgaagaccttttctatcgcgtcgagcggcagccgggcgcgcagctggatcaggacaacgcgtcgccgtccggcattcagatgggcagcggcacgcgcatcgtcggcacgcagaagatcttcacggacggcagtgtccagacgaccgggcagtcgctcgacgtcgcggtgatgggcaagggtttcatccaggtcctgatggccaacggcgagaccggctacacgcgcgccggccaattgagcatcaacgcggaagggctgttggtgaatgcgcagggcctgcccctgcagccggagatctcgattccggagaacgcggccgacatcgcgatcggcgaaaacggcatcgtcgccgcgacgatgcccggcgcatcgtcgccgagcgaggtcggtcagatccagcttgcgaacttcgtcaatccggccggcctgctggcgctcggcgacaacctgtacatggagacagtggcgagcggcccgcccaacgagggcgagccgggcacggacgtgttcggcaagatcaagcagggcgcgctcgaagggtcgaatgtgcaggtcgtcgaggaaatggtcgacatgatcgcggcgcagcggacctacgagatgaacacgaaggtgctgagcgcggcggacaatatgctgcagcaactgtcgcaggcggcgcgatga

SEQ ID NO: 108 - DNA, 657 bp, Burkholderia pseudomallei
atgatgcgcatcctcctcgcgttgacggggcttgcttgcctgggggcctgcgcgtcgatcgagccgccgcgtccggatttcgcggacgacgaccttccgccgatcgcgaccgcgcccgccgccggacgctccggcggcgtgttcaacgcggtcaccgcctggtcgctggcgtcggacggccgcgcctatcggccgggcgacacgctgacggtgacactcgaggaaaccacgcaggcgagcaagcgggccgacaccaagttcggcaagaacggcgacatggccgtcaagccgggcgtgttgttcggcagtgcgatcccgttcgattcgtcgttcggcgcgaaacgcaatttcgacggcggcggcagctcgtcgcagcagaacacgctgcgcggcgaaatcaccgtgatcgtcaatcaggtactggcgggcggacttctccaggtgaagggggaaaaggtgttgtcgctgaatcagggcgaggaggtgatgcgcttgagcggttacgtgcgccaggccgatatcgataccaataaccgcgtgtcgtcgcgccgcctcgcgaatgcacgcatcaaatacgtcggcaagggcgcgttgtccgattcgaactcggcgggctggctgacgcgcttcttcaacagcccatggatgccattctga

SEQ ID NO: 109 - DNA, 1179 bp, Burkholderia pseudomallei
atggatgccattctgatgaaattgagatttcgagatcattattcgagaaggcgcacgcgggctcagcgtgtgtgtgcgttcgcggcgatcctgtcgctgctgtccgtgctgctcatggcggcgtcgcgcagcagcgacgcggcgccgctcggcacgctggtgagcgtcgagggtgtgcgcgacaaccagctcgtcggctacggcctcgtcgtcggtctgaacggcagcggcgacggccagcagatccggtacacggggcaatcgatcgcgaacgtgctcaagcagttcggcgtcacgctccccgaaggcatccggttgcgctcgcgcaacgtcgcggccgtgatggtcagcgcgaatttcccggcgggctacgtgcccgggcagaagatcgacgtgacagtgtcgtcgatgggcgatgcgaagagtctgcgcggcggcaccttgctgctgacgccgctgcgcgcggccgacggcgtggtctacgcgctcgcgcagggcaacctggtggtgcccggcgtaagcgcgcaggggcgcagcggatcgagcgtcaccatcaacgcgaccgcggcggggcggattccgcagggcgcgacgatcgaacaggagatccccagcgacatcgacgcaaagccgttcgtacggctgagcctcaagcgcccgagttttcagacggcgaccagcatcgtcgccgcgatcgaccgcatggcggggcctggcgcggcgacgagccgcgacggcacgagcgtcgaggtcagggctcccgaagatcccaccgcacgggtcgcgttcctcgcgaagctgaccgcgatcaacgtgacgccgcagaaggagccgccgcgcgtcgtgttcaattcgcgcacgggcacggtggtgatcagccagggcatgaccgtctcgcccgccgcggtgtcgcacggcacgctgaaggtcacgatttccgaaggagcgatcgtcagccagccgaatccgctgggcggcggcaagacggccgtcgtgccgctttcgcaagtggacgtccagcaggacggcaaccgtatgttcaattggcccgcgggcgtgagcctgcagaagatcgtcgacacgatcaattcgaccggcgcgagcccggacgacgtgatggcgatcctgcaggcgctcgacgaagccggtgcgctgaatggcgagctggtcgtcatctga

SEQ ID NO: 110 - DNA, 351 bp, Burkholderia pseudomallei
atgtcgaatggaacgatcggcgcaggcgtgcggatggcgccggcggcgcggatgccggggctcgcggcgcaaccggccgcgaacgaaagcgatgagcgaaaggccgcgtatgcggccgagcagttcgaggcgctgttcgcgtcgcagttgctgcgcgagatgcgcaatgcgattcgcgagatcagccccgagggcagcatgtttcgggatgcggtcggcagcggcctgatggacatgatggacggcgcgatcgcacaggcgctcgcgacccagcgcgcgttcgggatcgcggatttcgtcctcgcccggattatgcccggcggcgatccgaccggcgccgccgcgcgctga

SEQ ID NO: 111 - DNA, 1359 bp, Burkholderia pseudomallei
atgaacatcatttatatcggaatgtccggcattcgcgccgcgaaccagtcgctgctcgcggccagccagaacacggcgaacatcgcgaccggcggctattcgcggcgcggcgtgttgttgtccacggggctcacgggcggcgtcgacgcatcgggcctgctgcgcctgaacgaaagctacaagacccagcagctctggcaggcggctggccagtacggccggtacgacgccgcgcagacctatttcgatcagctcgaggcgctgttcggcggcaatcctgttgacggaaaaacccaggcaaacgatctcggcttcggcaagttcatcggcgcgctggaggcggcgagcggcgatcccggcgattcggtgctgcgcaacggcgtgattgacgcggcgaattcgatgacgaagcagttcaacaacatgcgctcggtgcttaccaatcagcttcgctcgatcgagcagcagcgcgtcacgacggtcgagcaggtcaacggtctcgcgaagacggtcgccgagctgaacgggaagatcgcggccggacaggcgagcggcgcggacgtttccgcgttgatggatcagcgcgacgccgctgtcgacgacctgtcgaggttcgcgggcgtacgcgtcgtgcagcaggcgaacggctcggtcgacgtgtcgatggcgaacggccagccgctcgtggtgggcgcccaggccggcaagatcgaactgggccgcgacgcggccgggcaacagacgctttcgctcacgttcggcagtcaggtgatgccgttgcgcgcggcaagcctcggtggcgagctgctcgggctggcggaattcgaggaagaggtcctgcgcccgcagatggaggcgctgcgcacgctggccgttcgcacggccgagctgatcaacgatcaactgacgcagggtttcgatatgaacggcaacccgggcaagccgctgctcacgtatgacgccgcgaccggcctgctgggagtcgacccgagcgtgaagcccgaggagctgggcttttccggcgatccggccaatccgggcgataactcgaacctgctggcgctcgtcgagatcggctcgacgtcggtcgatttgcccggcatcggcgacgtgtcgctcggaaacgcgtttgcgacgatgatgggcaaggtcggcgcggcgagccagcagaacaagcaggcgaaaggcgcggccgaggcggtgcgcgggcaggctgaattggactggtattcggtcagcggcgtcgaccgggacgaagaagtgatgaacatgctccgcttcaaggacatgtacaacgccaacatgaaggtgatcgcggtcgccagcgagcttttcgaaaccacgctgaattctctctga

SEQ ID NO: 112 - DNA, 915 bp, Burkholderia pseudomallei
atgcgcatcacgagcaatcaatatcattcgatcatgacgcaggtgaaccgccaggcgactgcgggcctcatggacacgcagatccgcatggcgaccgaaaagcgcatcctgcgaccgtcggacgatcccaccggcgcagtgcgcctcgcgatgctggtgcgggccgacgcgatgctggaccagtatcgcgcgacgggggcgatgctgaacagccgcctgcaacacagcgagcgtgtgatgtcgggcatcgtcgatcagctccagaacgaggcggtgaagcacctgggcttcgcgatggacggcagccgttcgcccgaggatctgagcgcatatgcccaagtactcgcgtcggtgcgcgacacgctgctgcaatcggccaatacgcgggacagttcgggcaactacttgttttcgggcacggcgattacgaccgcgccgattcgattcgatccggccgcgcccgtgggcagtcgatatacgttcgaaggcaacctggagaagcagacggccgtggtcggagacggcgtgacggaggtcgcgaacgagagcctcgcggaaatggcggacgggctcaatgcgctcgacgaagcgatcgcgaagatgagcgagccgaatgcgaatccgaacgatccggcctatcgcgacgtgctcggacgcgccacggacacgatcgaatcggtcctcgcgtccgtcaacgcgaagatcggacggatgggcagcacgcaggcgagattgcagatgctcgacgaactgcacgaatcggcccagatcgtcgggaatcaggcggcgctcgatgtcgggaagatcagccccgaggaagtctttacggattaccagagctacatggtcgcgttgcaggcgagtcagaagctctatgcggaggtcacgcagttgacgctcttgaacgtgctctga

SEQ ID NO: 113 - DNA, 1068 bp, Burkholderia pseudomallei
atggaactgaaaacgacgatgcctgccggcggcgcgcgcgctgccgcggaggccgggctgcgcggcgggatcaggacgcccgccgcgatgcagcccgacatacgggatgggcgcgtgcgcgatgcatcgcacgcggttgtgcaaatgccgccggccggacgaggtgcggccgaccgccgggacgcgttcgcggtgcatcagcgtgtgaacgagcaggcatcggggatgcaactggtgctggactatctcgaacagttcggcgatcgcatgaaggctctgaagcaggcgctcagcgcacgtctggcgggcggcggcagcgatctcgacgtgcgcgcgaagatcgacagcgccacggccgcatggaaccggcgcgccgccgcgacgggtgggcgcctgagcgagacgctcgagctgggcggcgtggattcggcgcggcgcgcgttcgtcttgcgcggtctcgatgcgcaggacgcgcggcgggccgagggcgaggtcatcacgttccatctcggcggtaccggcgcacgcccggcaacgctgtatcttgcgccggatctgacggcgcgcgagatggcgtatcggctcaaggtcggccttgcgccgctcggcatcgattgcgaaacgcatgcacgggagcgcatcacgctttccgtcgaggaatcgcgctggccggtcgtcgcgcgcggactgcacgtaaagggcggcggcgtgcgctttcccgccggtgcgccgcagcgtgccgcgatggaagccgttgcgggccgcatcgacgcatcggactgggaggcgggcgatgccggcgcgatgcgggccgcgttgacacgtacggtgcagacggtgaagcggctcgatcaggtgtccattctcgccgagcacgcgctgaagcgggcgcggcagacgctggcgtcgatgccgtccggattgaatgcgcagtgggctcgggatgcgtgcgagcgcatcgccgcggacgcatccgcgcgcggcggctacggccaggttgtcgcgctgacggcggtgacggcgggtctcgatcgcgcgcgcgtgcgcgcgctcctgtcgatgcgttga

SEQ ID NO: 114 - DNA, 768 bp, Burkholderia pseudomallei
atgaagaaatacctgatagaagatctcgtcaccttcgatgcgcatctgatgacgctggcgcatctggaccaaaccacggaactcaccaccaacgaatcggaactgcttcaaatggtgctcgacgggatagccaccaaggccgccgtcattgagcgggtatggggcagcaaggggttcgtcgttaccgacagcagctatcaccagctcgtgcgctcggtgcgcaaccgcctcgaggaacacggcatcagcggcgcgctcatcaagacgctgcctcgtcaaggattgaggttcctgggttccgcgcaggcattgaccgatgcggccacgcccgtcacccagccggcccttgccgatcaaggcgagccggcagacgaagccgctctcgcgctcctcacgcagcatgagccgcaatccgcgccgcccgtcggtcgcgcgcggaactggaggcgcaggcttcgtttcgtggagcgcgcgctgctcgccgggctcgtggtctgggtcggcctcgtcacctggaagatgatttcgatcaacgatcgctttccgtggctgaccgggcatttcggcgtccatacccacgctcacgtcaccccgcgctccgccgggctcaagctgcttgcgtcgatcggcgtacggcccggccccggagagcgcgtctatgagatatcccgtaacggcgacagatggctcgcgatatgcgcgggaacccgacatagcagcgatgaacgctgcaccacctactccacccgggatctgacgacgtcctga

SEQ ID NO: 115 - DNA, 55567 bp, Burkholderia pseudomallei
gaaccgttgaaattccgtgcgatatcaggttttgaatgatcgcgggattgctgatcatcgtgagtgcgatcaggcgcgtgcccggataccgcctttggacatacgagaacagcgatatgccatctccgaaattgtcgtccggcatgctgtaatcggatatgacgacgtcgcaagggtgctcgttgagcgccgccagcagttcggtggagttcctgcacgcgcttaccacttctatcgaactaacgggagccagggcgtgtagcaagcctgtcagtacagccgggtgatcatccgcgatgatgacctttatggagaaagcagtcatcctgatcctgagtcatatgattggaagggattgcgtagaacaaaactaggatggagggcggtgcgatctattgatcatatgcatgtggcttgaggtgtcgaagcgattttcattgagcttgtcgattcaggggttcgatgcaccggaatttcgtaaaacaatggcccgatcgcatgggccgcggaaaaagaggcgcttcacaaaagtgaagcgccaattgagagctccaatgagatcacgcctcgaaggtcacgacatctcgacaaaacagctgacgcgataaattccctcgttgcgtgagtcatagagtaagtgacaagatttgtccgggctatcggacatgtatcaaagagataaagttgtcaatttttgacaattcgggttttccggatgcgatcccatttaggccggccatggccgattctgggtgaagcggccctgcgttcaccggtgtgcgttcgatgcacgcgtcatgtccgtgcgtgagagcggcgagcatcgcgcagaggggctatgtccggcggcctcgcgtgtccagtcgaccgcgccatgatggcggacgatcgctgctcacattccccttcgagggtggcaaacatgcggattgaatggcgcggcggctcgcaagggacgcgagatccggcgcgatcgtcgatcccgcggcacgcgccagtttcgaattttccgaacggcgcttgccgttcgcgacgagaagcgcgatggcgccgtgagcgtcagggcgcgcggcggctcggcatggattcgcggcgcggcgggaggcggaacttgatcgcgatgcacatgagcgcgtgaatgaatgtgtcatacttcgcgccaatcgcaaggacgagcaacgccatcgcacagcatgcccaacgaaaagcttccaacgcccagggcccgcgagcagttgagtgtcatcatgctcgatgatcatgaggtggtactgagcggtatcacctatcaactgaagcgggagcgcgacatcgccgtactgggcacattctccagatcgcgtcagttattcgattatctggctcagcatgggcccgccttggcgcatacccggcctgtcctgcttctcgactatgcgctcgcgccgcaggacacggacggcatcgcactgattcggcgcttgtgcgtccagttccccgtttgcccgatcctcatcgtgtcggggcaccatcagccgtcgatcgtcagattggcgttgatggcgggcgcacgcggctatatcgggaagaactgcacgctggaggaactcgtggaggcgattcggcgggtggcgggcggcgaaagatatgtgcatccgtctctcgcggaacgcgacgacctggtgctgcctgccgcgatcgaaatgagacgcggagaggcggagcgcgcgacgctggcgtcgttgttgtcgccgcgcgaactggaggtgttgagactgactctgcagggtttcgaagtcggacagattgccgagaaactgagccgaagcaacaagacgatcagcacgcaaaagcgtagcgcataccgtaagctcggcatttccggcgatcgcgagctgttcaagctcgagcgggaattgctcgacagttcgggcgacgcgggcagtccgtacgcgaactgacgcgtttggaggcgcggcacgcttgcgatgattgcgcgcgagccttgcgcggcgcacgagatgtttcagacatcgaacgagaggcgatccggggcaatgccggagacgagccgggcgagttttgccgcgttgcgccgcgccgcgttgacgttcacatcgggagtcgatagcgcgcgctcgagccgttccgcggtttgcgcgatgtcatgctcgcgcatcacatgcgcggcgcttttgatgcgatgcgcgcatgacgccgctcgcttgaagtcgccgcgtgcgagggccctgcctatgagcgcgctatcctcggcgcaggtggtcgcgaatagcgatgccagccgaggcagatcgatgtcccgttccgcgggcgacgatgagccgggggatccggcggagccgtcggccactccggcggcgcggcaccacaagtcgattatcgacgcgacatcgtcgagcatgagcggcttgcgcaaacttccatccatgcccgcggcgatgcactcatgcagatgggcatggtccgtcagtgcggaaatggcgatgaccggcagataggtcgttgcattccgccgggctgctcgaatgcggcgcgctacttcgtaaccgtcgatgtcgggcagattgcagtcgagcagcacgagcgcgaacgcctgatcatccagcagaccgagcgccgcttggccggtgttcgcgatagtcgcgtcgcaatgcagttcggcaagctgaagacggatcagctccgcgttgagcggctggtcttccaccaccagcacgcgcggccggtcagcgcgtgcgttggatggcgaatccgcgacggcgtcccggtcgacgggcgcgcatacctcgcgctgcgcggtgtcgaccggaatcactaccgttaccgaggtgccgacgcgttccttgctgtcgagcgccaacgtgccgcccatcaatgcgacgagttccttgcagatggtcagaccgaggcccgtaccgccgaagcgccgcgtcgtcgtcgagtcggcctgttcgaacgcgtcgaacaggtggtcttgccggttcttcggaatgccgacgccggtgtcgctcaccttcagcgtcagttctccgggcgcgtgcgtcggcggatcggcgatcgccagtttgacgtcgacgcacacgcgcccggcgtccgtgaacttgatggcgttcgacagcaagttgacgagaatctggcggatgcgtgtctgatcgatgtacacgtcgagatggtcgggggcgtcgagatgcagttcgagcggcaagcccttggcgtcggcctgggcgcgcatgatgatgacggcgctctcggcaacggtccggatgtcgctggcgacgtgcgcgagccgcagctttttcgcatggagcttggaaatgtccagcacgtcgtcgagcatgccgacgagactggcggacgccttgaccgccgtatcggcgagctgcttctccatcggccctagtttcgcgcggctgagcaggtcgattgccgatacgatcgcgttgagcggcgtgcggatttcatgactcatcaccgcgatgaagcgcgacttgcgcgcttcgctctcggccgtcaaccgatactgcgcacgggatcggaaggcgagcatcgcgaagcccgacacgacgaagacgccgaggacgatttcaatcgtgtagtagtgcgtgatcgcgcgcaaagacggcgcgccgaagtccgagctgtagagccagtgcgtcgtcagttcgttgacgtcgcgcgctgtcagcgagttcagcgacttgtcgatgatcgacgccagtaccggcaagtctttgcgcgtggccatgacgatcacggcgggcatgttcgcgacggcgcctgaaatgtacaggcggtgagaataactgctgcgaatcgcgggcacgagcatcgcgtcgagcccgatcgccgcgtctgcgcggcccgtcgcgaccgcgtcgagcatatcgcgctgctcgagggtcgagagtagtcgtatcgacggatggtgctgacggagaaagcgttcgtacgcgccgcccgagcgcactgcgatggtttggccggacagcttgtcgagatcgaaaatcacggtctgcgattcgcgtgtcacgatgaccgccgatccgacgagataagggctgctgaaggtcgcttcgcgttgaaggacgtcattctgcagaccataggcgacgttgggcagcaggtcgatttcgtgcgcggcgaacgcggcagtcggcgagccccagcgagtggtgtcgaccctctcgaactccagcccgctgatgcgggcgagcgcgtcgagatagtccgcggtaaggccgcggtgcttgccgtcttccacgtagtcgaagggctgcgcgccggggccggccgcgaaacgcacgacgggatgcgtgtcgatccagtgctgctcttcgctggtgaacagcgtggccgcgcgtgcgtggaacgccgcaaacggcgcgagcgcgatgctcagaccagccgtccagtgaagcaaccggcgtacacgagcagaccgacgggcttcaacctcgacggcgggcaggatcgagcaggcgcgcaggggatcgttcatgtttgtcgggtatcgcgaacgcgcaacggaacaggcggttcgcaacaggattggcttgcccgcatgataaacgcgcgcacgacgtggccgttcgttttcccgagtgaaaaaggggcgcattgcctgtcacccggcggtgcttgcatgccctcaaaaaaaaatttaagccttcgcgtattcgggtcgcctaacggtaatcgaagcgtaccgtgcgtgcgagtgcgaatcggcggcgccatgcgtcggctgcgtcgaggcgcgtgccgttgcgtcccgaggccggcgcggcttcgccatgacccatgcgatcggcgccgaacggcggttcgcgcgccgtgccgcgcggggttcacggcgtggattccgactcgattcaattcatcggaggattcatcgtgggtcacagggccgtcgcccggaagaacggacgcgtttcgcgcctggcgtcgaagctgcttgcgcagtcgccggtggtggccgttgacgcgtttccgtttgtcagtgtcgtatgcccgacgtggcagcgcagagcattcctgccgtatctcgtctatatgttccagtatcaggactacccggcgaaccggcgcgagctcgtgattctcgacgactccgcgcacagcagccaggatctggtggagcaactggctgccgccgatcccgcgggttcggtgattcgctattaccacgtcgccgaacgaatgacgatcggcgccaagcgcaatcgcctgaatgaactcgcgcggggcgactatatcgtctgcatggatgacgacgactattatccgccggacaaggtgtcgcatgccgttcgtgcgatgcaggagcgttgtgcgacgttctccggcagcgaccgaatctacatctggtacagccatatcgaccggatctaccagacctattcgttcggaccgcatcacgcgttgaacgggacgttcgcctatcatcgccactacctggcggaccatcgctacgacgacacggccgtattggccgaagagagcagcttcctcggctgcttcacggcttcggtgctgcagatcgatccgaagcgggcgatcctgtgcgtttcgcacagcgcgaatacgttcgacaaggatttcgttctggcgagctgcgagcgcgccccgttcaagctcgaggatttcgtcaccgatgtgcgtctgctcgcgcattaccgtcgcctcagccatgcgcccgtgagctcgcgagtcgaatggcggcgcttcgagcggatcgtcatcaacgccggccgcgacgccggaaggctggcggcatttcacgcattgttgctcgagcttggcgtcgacgagcggcaaatcgtcgagttccgttctgccagcggggacggtgccgatacgcaaagccacctcgatttggcgaagctggctcgcgccaacggctggcgcaattatctcctgctcgacgaccggctcgagttcgttcgccaagagaaagcggtggcgaacgtcaatcgctttctacgcgcgctggatggcttcgactgggaagtcgtcctgctcggcgcggatgtgcgaagcggcgtcccgttgcttaccctcccgggcgcgcagcgggtcaatctcgccggcgaagcggttgcctatgcggtcaaccagtcttactacgcgagtttcatcgatcatctcgaacgagtgctttccgtgcgtggcgcaggcgcgcaaggcgcgcacgaacggatcgatcaggcatggcttccgctgatggcgagttcgcgctggctggggctgtacccgagtttcgcctatctctcgcatcgcgaagacggaaaggatctcattcccgggttcttcaggaagatgcgggagaacgcagacacgccgtccggagcacctgcttctttcactccatcccgatccacatcatgaccgaaatcggaagctatgttccccagttcattttgcgcggcaaaacgcgcatcagccagtgccagtgcgcgagttgccgagtgcgatctccgctcgtgactcatgcatggaccaatcatgtgaggcagagcgcattgcttcagtgcgaaaccgtgtcgcgcgaggctctgtttgagcgcgacgcgttcgagctgcagacgattcccgcggacgtgggacaggatgccgagctcgatgcgtggcagaacggcgtgaatcaggcatgcatctatttactcctcgagcagggtctttcgattccggagcgactgcacgcgataggcgtgctgttaagccgggtatcgcacgccggcgacgcgcagcgcgatccggcgctgccgatcgccgtggtggaggaactgatcgggctggccggccacggtgtgctgcaggagcgcgtggcgcagatgccgatcatcgagcagtacaagcgcgcgtatcttcgtcgcctcgctcgcgtgagaatgtgtctcgatctcgacttgacgtcatcgatgttgctcaatctcaagctgacggaactgcaagtgatctcggatggctttctggctgacgcattgcacgagttcgagcaatcgcgggcagttgaaaagttcttcgataagcatgactacgtgtggcaaaactattttctgtacaggtgcttccacgaggtttttcccggcgcggatccggaactctatgatctggcattcctcgatatgtgtctcgactatttctgtctcagatcgctctgcgcaatactcgtatcgagccaagtcgatctggacgaagacgtcgtatcgagcctgttctgcgcatggcaacgcaacggcaagaccagcctgggccgcgacgatcacacggatccgctgctgatcggcttcacgttggtcggagaagtgcatgactgatgcggcagcgaatgcgaagacgtcgatgaacgatgaatgccgtatcgcgaacagacatgtgccgatcgattatcaggctacctatcgctttttcaagggccgtgcggccggcggcgcggacaaagtggtgcaggtactgtatcaagacgacaatcccgcgcttgcgcatgcgcggtccgaggcggaatgtcggatattggagcgagccctgtctttcgacgggcgttcagtgctggatatcggttgcggcaccggccggctggcggatcacatcatcgatcgcgtcggttactacttcggctttgatctgcttgcggaatttgtcgagactgccaaggcctccctcgtcgcgcgggccatcccggaaagccgctatggtttcatggcgctgccatgcaacgcccgttcgatcgaaatggtcacccgccgtaagaggttcgatatcgtgatgatgtcgggcgtgtcgatatacctcaatgacgaggattttctagggagtcttcgttgcatcgcggcggcgatgcattccggcgcgagtctgtatctacgagatccgatcgagacggccgacacgcgcctcacgctcaatcggttcatgtcgaatcagttgtcggcggaatacagcgtgatctatcgcggtgccgatgagtatcggcgaacggtggccgaggcattcgacgcgtcgtacgacatcggaccgctgacgcctatgtatcgggaccctgcgctgaacggcggctggtcgacccggcagcactacatggtgattacccggaaatgagcgatacgctgatttgcttcgatctggacggcacgctgagcaagcaggagattcttccgagaatcgccgaactggccgacatatcagaggaaattgcggcactcacgcaggcgacgattcaaggcgtgattccgttcgagatgtcgttcaagctgcgcgtacggctattgcgcgacatatgcccgagaaagatcagtgattacgtggccgagacggtcgagttggacgagcgcattctgcggtacatccggaatggaagcggggcggattgcgtggtcgtgacgggaaatctcgactgctggatcgaggggctcgtgcgacgcatcggcgtgccgtacgtcagttcgctgggcgaagtgaagaatgggcggctgctgggcgtcgcgaacgtattgcgcaaggatgcgcccgttgcgcaattccggcgtgactatcggcggatcatcgcggtcggagacggcgagaacgacattccgctgttcagacaggcggacgtgggtatcgcatatggcggcgtgcatgcgccatcggccaatctcgcgaacatggccgactacatcgtatattcgagcgaggcgctatgcgacctgttgagcatgcagtaattccgtgcgcgggcgtcggctcgcgattgaatcgcggcataccgaaatcgttgacggaaatcgacggtcatacgttgctcgggcgcacgctgcttgcgctcgagcacattcctcatgtgtttctcgtggtcgggttccgcaaggaactggtcatcagcgaggcactgcgatatcgccgcaacctgatcttcgttccaaacgaatacttcgaccggacgaatacgcaatatagcgtcaagctggcgagccggctgatccgggacgatttcctgatggtcgacggcgatgtcgtgttcgacccgtcgtcgtttgccgcgctgctcgaacgatttcaggcctgcgatggccccgcgatcgcctacagcagaagaaccaccgaggacggcgtcaaagtccatctcgaaggaacgcgcgacgaaacgacatgcaccggattctttcgcggcgaaggcgcccatgaatggacgggcatcggccggctgaacaatcgcgtgatctcgggcgactcgctattcgtctacgagtcgatcgagcggcacatgcccgcgccagcctacctggtggattcgatggatatcgatacgcctgccgatttggaaatcgcgcgagcgacgtatggccgtcatgaccgttaagaagaagatccgctttctctttcaggcggcgagccattggtactgctttgcctccgtataccgggcattcggcaacgattgccgttacgacgtcgaagtcgtcgcgttgccctttctgaagaatgacgttcagctcggcgagccgtcgtgggcatttctcgaggcgcaggggattccgtttgtccattaccttcaatacgacacggatgcggacgcgcccgatgtcgtattcctgcattgtccgtacgacgagacgaggccgccggcatttcatgcggccgcgttgcatgagcacaacgtgcggctcgcgtatatcccttacggccccgatgtcggcggcggcgagcgaaatcgccggttccagtacgggctcgatacacaattgctcgcatggcgcatcttcgcacgctcggaagcgcatcggatgcgatatgcgcgcaacttgccgggcagtgcgaagcgcgtcgtcgtcaccgggcatccgaagctcgatgagcttcgcggcgcgcccgccgacaacccgttttcgccgctggtggacggcaagtgcgtattgctttggaatccgcatttctctgtgggctttccggcggagctgcgttggtcgacgttcgatcgctatgtgaacgcgatgtgccggctcgccgcgcagcgcgacgacgtcgcgatcatcatgcggccgcatccgaacctgttccatacgctgatgctgcgtgagccgggaaagcggctgatcgcgacgttgatcgcgttcgcgaatcggcatcgcaatttcgtgatcgatcaatctccgctgtacaaagcggctttcgatgcatcgcatgcgatgatgacagacacaagttcgctcatgtacgagtacctcgcctgtgacaagccgattctgcatctcgattgcgacggcggcgcgggactgaacgaggagggcaggcaactggttcgcgggatgtcgcgcgcgcgtgcggaacgcgacatcgttgcattcgtcgacgatatcgtgcgcggccgcgacgtcacgcaggcattgcgccggcgcgtgatcgccggcgtactcggcgagtgcgacggcaagaatggtgaacgcgtgaagcttgccgtcgatcgttcggatctctggcaatccgtgcctgcttgagcgttgcatggctcgatgcgatttaagtttgtcaatggaagcgacgcctaagcatggtgagggcgccggacagcgccccgctccctgatagaccattcacacaggaattcagcatgcttagtctgcacacgaacatcgcttcgatgcgaatccaaggcgccctcgggcgcactcagcaggacctcaccacgtcgatgacgcgcctcggtacgggctaccgtatcaactcggcatccgacgacgccgccggcctccagatcgcgacgcgcctgaagtcgcagcacctcggcatgggcgtcgcagctcagaacacgcagaacgcaacgtcgatgctgcagacggcggaaggcgcgttcaaggaagtggagaacatcctgcaccgcatgaacgccctcgcgacgcaggcggccgactcggcatggacgcaggctgaccgtgactcgctgcaaaaggagtacgacgcgctcggcacggaactgaagaacatcgtcgacaacacgtcgtacggcggcgagaagctgtttgccgccggcgcctccggcgcgccgggggggaagctgacggaaaagctgacgttccaggtcggcaatacgtcgggcgaagtgatggagctggacgtgtcgacggcgctcgcggcggccgtaaaggctttcgagggtgcgtcccaggtgttcggcggcggcgcgtccggcgcgtcgggtaacaccgacatcacgagcattagcggcgcgaatgcgctgctcgacaatctgtccaccgcgctgtcggcggtgggcagcctgcgtgccgagatcggcgcgggccagaaccgcctgacgcacatcgcgaacaacctgacgaacatgcgcgacaacacgcaggacgcggaaggccgcattcgtgacgtcgattacgcgagcgaaaccgcgaacatgaccaagctgaacattctgcagcaagccggcggctcgatgctcaagcaggcgagccagatgagccagctcgtcctgtcgctcctgcagtaagcgagtggcgggaaggaggcgccgccgcgcaggtggcgccacgggccggacccgcatcaggcatggtgatgagggagccggctcgcccgcgcggatcggagcgattcgatgcgatgaatcttgtcgacgtttccacttcgcttcgcgcgccatcgccgttcttgcgggcaacgcacgcttcgcgcatcgtgcgggatgcgcgctttcgtgcgcggcgggtagccgcgtcatacggcgcgcttatccgcgcgcccgctatctaacagacatttaaggatttccgttggccggacgcctgccgtatcggcaaagcccggatcgtcgagcgcggcggccggagcgcaacgatatcgaatcgaacgtgatgcggcagcggcgctgcgcgtattccgaaaggcggtgaagagtctatgcaattggcaattgggatcctactggtattcggctgcgtgttcggcggattcgtgctgcatggcggcgcgttgagcagcatctggcaaccgaccgaaatcctgattatttccggtgcggccgcgggcgcgctggtgatgggcaatccgcgcagcgtgctgtccgaaatgggctcgcagatcaagcacgtgatcgcccgcaagagcaatggcgtcgaattccagcgcgagttgttgctgatgttgtacgaattgctcgagacggtgcgcgacggcggcctgaaggcgctcgatcagcatgtcgaggagcccagggaaagcgatctgttccgcaagtacccgatgattctcgcgcaaccgcgtctgattgcattcatcgtcgacaatttccggctgatggcgatggggcgcatcacgcagcatgagctcgaagggatgctggatcaggagctcgaggcgatcgagagtgagttgatgcagccgtcgcgctcgctgtcgaaaatcgcggaatcgatgccgggcttcggcattctcgcggccgtgctcggcattgtcacgacgatggccaacatcggcgggagcgtgtcggagatcggtctgcaggtggccgccgcgatggtcggcacattcatcggcatcctgctgtgctacggcgtgctggagccgctggccagcgcgatggtgcatcgcgtgaagagcgacatgtcggcgctggagtgcgtgaaggtggtgctgtgcgcgcaagtcgcgggcaagccgacacttctcgccgtcgatgcggggcgcaagctgatccagctggacgtcaagccgagcttctcgaagctcgagtcctggatcatggaaatctgacgtgcgtggatcgcgacgatgagcacgaacaaggaatcggggcacgggccgacgatcatcaggcgagcccacaagcgccacgaggagcacagtggtggagcgtggaaagtcgctttcgccgacttcacgttggcgttgatggcgctgttcatggtgctgtgggtgatgaacgtcacgccggagaaggagcgcaagcaagtggccgccgaaatcgcgggacggccttttttcgacggaggcgtcggcatcttcgagcagcgcagccgcaagccggtcgtcgcgccgttcgatgcgcagcagcccgatcggatgaggcgccggacatccggcgcggcgcgcgagcgcacgatcgattcgcttcagcagcgccgcgcgctcgccgcgcgcatcatggacgaggcgagcaggctcggcatggacggcaacgtcgccaccgcgatcaacgacgacggcgttcgcatcacgattcacgattccgacaagcagggaatgttcgagcggcgcagcgatgcactgaatcccccgttcatcaggctgctcacggcgctcgtgcccgtgctcggaaccgtgacgaacaagatggtgatcgtcgggcataccgatgcgacgcaatacgccgacgcctccgtgttcagcaacaactggaatctgtcgagccgccgcgcgctgcgcgctcgccaggttctcgccgacggcggcatggacgcgcagcgcatgtttcagatctccggcatgggcgacagcgtgccggccgtgccggacaatccgaatcacgacatgaaccggcgggtcgaactgctgctgctcaccagccgcgccgaagacacgtggcgcagattgtttcgcagcgaggatgtcgacgtccgaccgagtgcggacggaatcggcttgacggtggggcaggcgcagcccggcgcggatcggcccgcgtttgccagcgagccggttcgctgatctcaccgtggccaacgtgaagaagattctctcggtcgacgacacggtcaccattcggcggctgatcgcgcacgtcctcacgacttcgggctacgaggtcgacgaagcgcaggacgggtgcgcggggctcgaaaaggcggccaagaacgactacgacctgattctcaccgactacaacatgccgcacatggacggcatcagcatgttgcgggcgattcgggcgctgccggcgcatggcgcgacacccatcgtcgtcgtcacgacggagttcggcgccgatctcaagcagcaagggcgcgatgcgggcgccacgggctggatggtcaagccgatccatcccgggaaactggtcgagatggtccgccggttgatcggttgaacgacggaaagcggtacatcagcgatgcatggcggaaagaccgaatgtcgatagatttgactcaattcagcgaggtgttcttcgaggaagcggaggaacacctgcgcgcgatggaagcgttgctgctcgcgtgcgacgtcagccatcccgatgccgaggatctcaatgggatcttccgggcggcacattcgatcaagggcggagcgggggcgttcggcttcgccgaattgaccggcgtcacgcatgtgctcgaaacgctgctggaccggatcagaaagaaggagctgccgctgtgcgcggcgatggtcgacgcgtttttgcgcgccacggatctgcttgcggtgatgctcgcggcgtatcgcagcggagaggcgctgcccgagcagagtatcgaggcgacgaagcgcagcctcgagcgcctgagccaaggcgaaggcgcgcacggcgccgacgccgtcatgcccgatgcggaacccgtgccggcctcgtccgagcatgacggcgacgcacatccgctcccggaagttctcggcgacgcatatcggaaaaccagtccggcagtgatgccgccgcccggctgcctgcacgtgcggatcgcggccggcgctccgatcgacgacgaaggcgtaatgtcttgtctcgcgagctatggccgagtgatcgagcattatccgggaggctgcgatcagccgtggcgttacttgtgcgagacttcccgcgacgcagccgacattcacggcacgctgacgttcctgatcgatccggaccgcctgaccattgatcaagccgcgcccgaaggaggcgcgctgccgccggagtgggaggaggagcatgacggctatggcttcttcgtgcccgtgccgacgatcggcgacggcgacgcggcacatgaagccgaggcgcgcgatgaagccggccacgcgcgcgccgagactgggcgctccgatgcgcaagcgaacgcatccgcgggcggttcgtcggctgcatcggctggggtgccggcgctcgcttcgccgtcgccttcccgatcgaggcaagcgggcgacgccgccggcggcgacagcatggccaagcgcaacggcgcgggcggctcgtcgattcgcgtcagtgtcgagaaagtcgatcagttgctcaatctggtcggcgaactggtgatcacgcaatcgatgctcgcacaggcggcgatcgagctggatgcgaatcagcgcgagcggctggtcggcgggatcgggcagatcgaacggaattcgcgcgagttgcaggaagccgtgatgtcgatacggatgctgccgatctccgcggtgttcaaccgcttccctcgggtcgtgcgtgatctggccatgcagctcggcaagcaggtcgaactgcagatgatcggcgagcacaccgagctggacaagggattcatcgagaaactcgcggacccgatgatgcatctcgtgcgcaacagcgtcgatcacggtatcgaggcaccgcaagtgcgtgtgcgccgcggcaagcccgcgacaggccggctcacgctgcgcgcgtcgcagcaaggcagcgacatcgcgatcgaggtgatcgacgacggcggcggcctccagcgcgaacgcattctcgagaaggcgcgggaaagcggtctgccggtatcggaatcgatgaccgatcgcgaagtctggatgctgatcttcgagccggggttctcgaccgccgcagccgtgacggacgtgtcgggcagaggcgtcggcatggatgtcgtgcgccggaacattcaggcgatgggcgggcggatcgacgtggattcgacgccgggcgtgggcaccgcgatctcgatcaagttgcctctcacactggcgattctggacggcatgtcggtgcggatcggcggctcgcgctacatcgtgccgctcgcattcatcaccgaggccgtcatgcccgcggatacggacatccgcacggttcagcgcagcggacgcgtcgtcaacgtgcgcggcgaatacctgccgctgcgctcgctcgccgatctcttcgagacgggagaagacaccaccgacggcagcctggcgctgattctcgcgtgcgactcgcaaaaggtcgcgctgctcgtcgacgaacttctggggcagcaccaggtagtggtcaagaacctcgagaccaactaccggcgcgtgcgcggggcggcgggcgcgacgatcatgggagatggtcgcgtcgcgctgattctcgacgtcatgagtctcgtcatgtcggcgcggaattcggcaaacgtttcggaagcggaaagcgtgtaggaggctgcattgattgaaacggaaaacacccagcgcgagacgctggtgttcaggctgggtgcgcaggagtacggggtcgacatcctgaaggttcaggagatcagggggtacgacgcctcgtcggtcacgaacatcgtcaccgagccgcattacatcaagggcgtcatcaatctgcggggcaacatcgtgcccgtcatcgatttgcgcgggaagttcgggttgggcgacgcgcaatacggcggcacgacggtcactatcattctcaacgtgtccggcaggacggtgggaatcgtcgtcgacggcgtatccgacgtggtggtgttcaagcgcgaccagatccgtcccgcgcccgcgctcggcgcggcgatccgggcggagtacgtcgacggtctgggcctgctggacgcgcggatgatcatcctgctgggcattgaacgcctgctcatgagcggcgatttcgaactgccgccggcagtggcttgagaaacgaatcgagagagagcatgacattcaatcagaagatcatttccgcattcgcggtcatcatcgcgggtgtgatcgggatgctcggcatggagatgtggtcgttgaacaacatcgaaagccgcgtgacggaactgcacgatcagcgcctcgcgacgctcaccgtgatgagcagcatcgaggacgatgcgctgaaggtgtcgctcggcatcgcgcaggcagccgactacatatgggtgaagaaggtcaaggcgatgagcgcaggcggggtggtcggcatcggcgaagaggcggtcgaccgcaaatcgttcgagtcgcacgtcgaaggcgcggagcgcgcgcttgcccgcacgatcgagaaactcgagcacaacaagatcaaccgcgaggggctgtccgccgacgaagctgcgaagattgaacgcatccgcgtgcagctcgcgcaactgcagccggatgcggtcaaggacgcgcgccgtatcctcgatatcgcggcgagtggcgaaagcgatccggacaggtatctggcggcttacctctccatgcggtggcagccgttttacgagaaggtttcggaactgtcgagggcttcgacggcggccacgagcaagagcatcacggacacgctggataccgttcgcgagcgccgcaggcacgtcgccgtcgtcagcctgttcgcgctcgtggtgctggccggcatcgtcgtctggcagatctggtacatcaagcgttcgctcggcggttcgatggaagcgctgtcgcggcacatgacatcgatcgcgcatgacgatctgagcggcgacatcagtgtggacgagcgcgacaagcagagcctgatgagctcgctggccgtcatgctcgagcgcttgcgcggagccaaggcgatggttgtcgaaaacgcacgcattcgcgtcgcgctcgactccggctcggccaacatcatggtcgtcgacgattcgcgcaaggtcgattttgcgagccactcgctgatgcgggcgtttgccgcgatgcagggcgcattgcgccgcgaggcgccgcaattcaattcgaatcagatcgtcggcgcgctgatcgacaccgtactcgagcgtcgcgtcgcggacgaactgctcggcgcgagcagccagcagcgctgcgaggtcaaggtcggcggccgcgactggctgctcgtatcgagtgtcatcgtcgacaacggcaaacggctcggttttgtgatcgaatggatcgaccgcacgaacgagttgcagatcgagcgcgaagtcgaagccgtgatcgccgcggttgccggcggagacttctcgtcgcaagtggcggaagcgggcaagacgggctttcatcagatgcttgcccagcggctgaaccgcctgacgaagaccgtggcggacgtggtgacggaagtggccacgacgttcggcaagctggctgacggcgacttgcgcacgcgcatggcgggcgcttaccaaggcgtgttcgccgaactgaaggagaactgcaatcgaagcatcgcgcagctcgaacgcatggcgatccgcatccgggacggcgcgcacacgatcaacggcgccgcgcgcgaaatcgcgtcgggcaatgcgaacctgtcgtcacgcacggagcaacaagcggcgagcctcgaacagacggccgcgagcatggaggaactgacaagcaccgtcaagcagaacgcggagaacgcgcagcaagcgaacacgctggcgctcaacgcatcgtcgatcgcgcagcggggcggccaggtggtcggccaggtggtcgagacgatggggcgcattacggcgagcgccaagaaggtcgccgacatcaccaccgtcatcgacggcatcgcgtttcagaccaacatccttgcgctgaatgccgcagtggaggcggcgcgcgcgggtgagcagggacgcggcttcgctgtcgtcgcgagcgaagtgcgcaatctcgcacagcgttcggcggcggcggccaaagagatcggcgcgctgatcggcgaatcggtcgtcgagaccgattccggtgcggcgctggttcagagcgccggcacgacgatggacgagatcgtgaatgcggtgtcgcgcgtcgtcggtatcgtcggggagatctcgagcgcctgcgtcgagcagagcagcggcatcgagcaggtgaatcaggcggtctcgcatatggacgagaacacgcagcagaatgccgcaatggtcgagcaagctgcggctgcggcggaaagccttcaggagcaggctcaactgctgcacggcacggtcgagatgttcaaggtcggcgaacggccggctatcgacgcgttgccggaggtccgagccgtggagccgccgcccgcggtgccagccgtgacccgcaggcgcgctggcgccgcatcggcggccgatgaatggaccgacttctgaccgggcgcgcgtgaattgccgggccgcctcaccgccacgccggcatcgattgcacggcgcgacgttcgcgttacaagaacgtcgcgcctccatcacatacagccggacccgatgaatagcgtggattttgaatacacgaacgcggatttccgccggattgcacggcatgtctatcagcgcgcggggatcgtgctgaccgaggcgaagcgctcgcttgcatacgggcgcctggtacgccgcttgcgtgtgctgggtattgggcgcttttcggactatctcgaccaactcgaggccgatcggtcgggtgacgaatggcagcatttcgtcaacgcgctgacgaccaatttgacatcattctttcgggagagctaccacttcgaccttctcgcgacgcagatgcgggcggcttcggggccgttccggatctggtgcgcggcggcaagcacgggcgaggaaccctactcgctcgcgatgaccgcgtgcgatgcgcgcgggacggcggcgaaccgcgtggagatcgtcgcttccgacatcgacacgaaagtgcttgccgcggcgcaagccggcatctacaagctgcatcagctcgacaagatgccgcttcgccgcaagcaggcctattttcagcgaggcagcggcagcaacggcggcacggtgcgcgtcaagcccgcgctgcgcgagatggtcacgttccggcagatcaatctgctgacggaatcgtggccgctggacggctcgttcgacgcggtcttctgccggaacgtcctgatttatttcgacgccccgacgcagcagaaggtgttgcgcaacatggcccgccacctgaagtcgcacggccgcctgtacgtcggccactccgagaacctcagggccctcgacgatctctacgcaccgtgcggccgcgcagtctatgagctcgtgaatgggcgttgacggtgtgggctatcacttctacttcgaccgcgaattcggccggcaggcggtacgcgtcaacccgggcggcttcgcgatcgcttcggcggacgtgatgctcgcgaccgtgctgggttcatgcgtatccgtttgcatgttcgattccgccgcgcgcgtcggcggcatgaatcatttcatgcttcccggaagcggcaacggcgcgcggcacgacagtctgtcgtccctttatggcgtgaacgcaatggagcggctcatcaacggcttgctcaggcgcggcgcgctcaaatgccggctgcgcgcgaagctgttcggtgggggctgcgtgatgccatccctgagcgacacgcgcatcggcgagcgcaatgccgcgttcgtgcgcgcctacctcgacgcggaaggtatccgttcggtcggcggggacatgctcggcacgcggccgcggcgcgtgtgctatttcccgttgaccggccgcgcgctgtgcaagcggctggtttgcggaagcgatgtcgccgatatcgcgacgagcgagcgggcatatgacggcgacctggcacggcggttgcccgtggccggcagcgtcgaactgttctagccgccggatcggccgggggcgatttccgcgcggtcgatttcggctgcgaaacaacaagggaaacgaattgattcgcgtagtggtggtggatgattcgatgtcgatgcgaacgttgctggaacgcatcatcaatggttgcgacggcatgacctgcgttggcgcggcgcaagacgcgaacgcggcgcgggagatgatccgcgcgctcgatcccgacgtcgtcacgctcgacgtcgaaatgccgggcatggacggcttcgaattcctgcggcgcatgatgctcctgaagccgaccccgacgatcatggtgtcgggacgtacgacatccggctccgatgccgcgctgcgcgcactcgagcttggcgcagtcgacgtgatcgccaagccggtgttgaccgggccggccgatcttgccgactatgcgcgcgacatcgcggagctgatccgcggcgcggcggccgcacgcgtaaaaggcggtgtgctcgccgccgcgtcgcccgcacgaggcgagcgtgcgtgtattcaacggctgcgcggcgcgaagaaggtcggcatcgcggcaacgcgtctgagcagggtgatcgcgatcggtgcgtcgacgggcggcacggaggcgctcagggccgtgctgcaggacatgagcggcacgccgccgccgattctgatttgccagcacatgccggaggggttcacggcttcgttcgccgcccggctcgacacgatctgcggcattcgcgtcaaggaagcggagcagggcgagccgctgcgctacggctgtgcatatgtcgcgccgggccactcgcatctatccctggcggctaccggccgccactacgtctgccggctcgaggcgtcgccgcccgtcaatcggcaccggccgtcggtcgacgtgctgttcgattcggtcgcgcgcctcgcgggcaagcgcgcgctgggcgcgattctgaccggcatgggcaaggatggcgcggcggggctgctgcgcatgcgagcttcgggagcacggactttcgcgcaggacgagcaatcgtgcgtggtgttcggcatgccgaaagaggcaatcgcgatgggggcggtggatgaaattctgccgcttgcccgcatgggcgcgcggctcagggaagcgttgcaatgacgtaccacgccagccgggcggcttttgacagaattctcatcgaaggaacgataaacgatgattcgcgtactggtgattgacgattccgcgacgatgcggatcttgctgaagaagcttatcgacaagaacgaacacatggaatgcgtgggcgtcgcgcccaatccggtggcggcccaggatctgcttcgggaagccaggccggatgtgattacgctcgacatcgaaatgccgaagatgaacggcctcgatttcctcgatcgcatcatgcggctgatgccggtcgcggtgatcatgatctccacgctgaccgaggccggttccgaatcggcgctgcgtgcgctcgaactgggcgcgatcgatttcatcgcgaagccgaagctcgatttcgccgaaggcgtgcaggcgtatgcggaagaaatctatcgcaagatcgaaacggccgggcgagccaaggtgaagaagctcacgcgcgacgtgccgcccgtgcgaatggatgccgaaccgcccgccaagccgttgctcgcggaggcgggcacggaaggccgcgtcgtggcggtgggcgcctcgacaggcggcaccgaggcggtgaaggaactgctgttgggtcttccggcggattgtccgccgctgctgatcgcacagcatatgcccgagccgttcatgcgttcgctcgcgaagcgtctggacctgctgtgcgcgatgcgcgtcaagatggcggaagacggcgagacgctgcgtcgcggttgtgcgtatatcgcgccgggacattcgaacttgaccgtcgaggcgacggcagccgggtatgcatgccggatcgtccggaatgccggcgaggcgcaggcgggctcgtccgtggacgagttgttccggtccgtggcggccgccgcgggtgcgcgcgccgtgggcgtcgtattgacgggcagtggctcggacggcgccgccggcgcgcgcgcgatgatggcggccggagcgttcaacattgctcaggatgcggaaacgagcgtggtctacagcatgccggacgcggcgatcgccgcttgcggcatcaacgaagtcctgccgctcgagaagatcgccggaaagctgatggaactcgaccgcgcatgaggcggtgcgccgagcgcctgcggacggcgccggcggtgcggcgcgagcgggagacggacgtcgatgcgttcgccggagcgcacccgtgcggcctgaaacccgcgattcggcttcctttgccgggaagagcgaggaagacggcgcggcgcgagcggccggccgccgccgccgatcattcgagcgtgccggcgatctcgatctgacgttcgtccgggatctcgttgtacgacagcacgtgcagcccaggcgcgaacacgcgcgcgtagcgcgcgaggagcggccgcagttgcggcggcacgaacaggatcggcgcgtgcccgagcgtgcgcatgcgctcctggacgccgggcaggctgctttgcaggttcgtcagcaggtcgggcgcgatcgcgaagttgtcgagcggggccttggcgctctgctgggcttgattgaacgcggcgagcagaatgccctccagctcgccggccagattgaagacgggcacgctgcgccgatggccgacaatgcccgcgacgatctcgcgccgcagcgcgcagcgcacttccgcaccgagcaggatcgggtcctgggtcgcttcagcattctccagcagcgccgtcgcgatcgccacgatgttcctgagcggcacgcggtccgtcagcagcacgcggcacactttgcggatcaggttatgcggcatcgctttctcgagcgcttcgagcaggcgcggcgcgacctgaccgagccgctgcagcagcgatccgacatcatcgtgggtcagcagttcgcctagatggtcgcgcagcagcttgtcgacgtgcgtcgcgacgacagatgcgccgtcgaccacctgatagccgaggccgatcgcgtgtgctttgtcctgcggccggatccacgtcacggggatgccgtaggcggggtccgctgcgggaatgccgtcgactgatccgtacgtcgtcggcgaaggtatcgccatcagatgatccggatgcagttcagcgcggtcgatctcgacgccgtgaaggtggatcgcgtagtgacttgatggcagggacaagtcgtcccggaccgtaatcggcggcacgagaatgcccatcgattcgctcatcgtccgtcgcatcccgtcgacacgccgtcgcagcggcgcgccgtgcgcgggatcgaccagatcgacgagcctgtatccgacccggatgccgagcggatgcacgtggctgatgctgtcccagtcgagatcctgcacgttcgattcgcgcagcgccgtttcgagcgcgtggatctgcgtttcctcgcgctcgggcgccggccgtttggcgacgcgccacgcgacgtagccgagcacgcccgcgaatgcgctgaacggcaaccacggcatgccggggatgatcgccaggatcatcatcaccgccgccccgctatagagcacgttcggcgatgcaagcatctggcgcccgacctgttcctcgaagtcgccggaatcgctcacgcgcgtgacgatcacggccgacgcggcggcgagcaggagcgacggaatctgggcgacgaggccgtcgccgatcgtgagcaggccatatcgctggaatgcctcgccgaaagcctggccgtgagcgaatacaccgatcgccaggccgccgagcatgttgatgagcaggatcagaatgccggcgatcgcatcgccgcgcacgaatttcgacgcgccgtccatcgcgccgtagaaatccgcctcggtagtgacgtcgcggcgccgcgcttgcgcctgctcctgattgatcaggccggcgttgagatcggcatcgatcgccatctgcttgccgggcagcgcatcgagcgtgaagcgggccgacacttccgagatacgctcggcccccttcgtgaccacgacgaagttgatgatcatcaggatcacgaacacgaccaggccgacaacgaagttgccgccgatgacgaccttgccgaatgcttcgatcacgtggccggcggcgttggtgccctcgtggcctttgagcagcacgacgcgcgtggacgccacgttcagcgtcaggcgcatcagcgtggtggcgaggatgatggtcgggaagaccgagaattcgagcgggcgcttgaccgacacgctgaccaggatcacgacgatcgcgaggacgatgttgaacgtgaagagcagatcgagaatgacgggcggcagcggcaggatgaccatcgacagcaccgccagcagcaggatcggcgtcgcatattgatactgtctcaagtcggcgaacaggtgtgtcagtttgttcatgagggtcgtgttctgtcgagaaaatccggaatcgcaacatcggtcgggcgttcggggcgcaggccgcggcgcccctgcctgaacgctttcagttgcagcacgtaggtcagcacctgggcgaccgcgcgatagagcgcggcgggaatttgctggttgacctggctcgtgtcgtagatcgcgcgtgcgagcggcggcaggctcagaacctcgacaccgtgttcttgggcgatccgccggatcgcgagcgccatgtcgtcgacgcccttcgcgatcacgaacggcgcttcggctcggttcgtgtcgtatttgagcgcgaccgcgtaatgggttggattgacgaccaccacatccgcgctcggcacggtcttcgacagtacgcgctgagcggcttgccgctgaagctggcggatgcgcgagcgcacttccgggcgcccttccgcgttcttgtgctcgtccttgacttcctgtttcgtcatccgctgtccgcgcaggaaaacgaagcgttggaacggcacgtcggcaagcgcgaagacgagcatcacggcggcgagcgcgagcgcgccgtcgagcgtgaggccgaagccgcgcgtcaaggcttcctgcagcggcaagtgctgcagccgcagaaatgcggcgagcgtcgtcgcgctgagatacgcaagcattgcgatgagggcgagcgctttcagcagcgatagcgcgaaggtgccgtagtggcgcgccgagacgaggcggccgagattcgtcatcggattgagccgcgagaacttcggtttcaactgcgcgccgctgagtatccagccgccgggcagcagcgaaccgagcacgacgcaaagtggcacgacgaagaatggcgcgatcatcttgaggaacagcgcgatcgataccggcagcgcgaccgaccacgcgttggccaatgcgccgtcgccggtgacgtcgacgagctcgagcagaaacagcgcgcgaaactcggcgagccacgagggcgcgagcaacacgagcatcttcaatgagacgagtacgccgatcgcggtcgacaggtcgcgcgagcgcgcgatctggccttgcctgcgcgcgttgcgcagcttttgcggcgtggctttctcgctcttgtcgccactgtcctggttagccacgcgcggctcccagatgatgctcgagcacgtcgagaacgtgccgcgtcatgctggcgtaatgcgcgggcaggcccttgagcatggcgccgagcatgagcagtccgaatacggtcatgatcgcgaagccgagggaaaacagattgatgctgggcgtcgcgcgggacagatagctcttgccgatctgcaagacgaccgtggcgaacacgagcggcgccgcgaggagcatcgtggtcgagaagagccaggcggcctggtaggcgagcggcttgagcgaaggcagatcgatcggcgtgccgacgggccagacgcgaaaactcgtatagagcacctgaacgacgacgaggtgcgcgtcgatcgcgaagaacagcaggatgcttgccacgagcatgaagccggacacgacatccgacgtcgcgccgttgagcgggtcgttcatcactgccatcgcgaggcccatttgcgaggcgacgaggtagccgagcatcatgaagacggcctgcacgaagtgaaagagcatgccgaacagcaagccgatcaggatctgctcggcggcggcgagcgcgccgcgggcggagaagaccggaatggcgacggggtcgacggcgggcagcgcgatcagcgcgaggaacagtgcgatcgtcacgcgcatccgcgcaggtacggtgctttcgccgatgaacggcgctgcgctcaacgcggcgagaatccggcagaacggccaccatgcccgtgcgagccactcggcagcggcggcgacgatggcgtccatcccgtcagccgaccagttgtgccgcgcgcgtgaaggttgcgacgcagtaatccatcagtgtctgtacgagccagtgcccggccagcacgagtatcgccagcgtcgccagcaggcgcggcaggaagctgagcgtctgctcgttgatctgggtcgcggcctgaaacaacgcgacgaccagcccaaccagcagcccgggggcgatgatgacggcgaccatcagagtgacgagccggatcgcattgccgacgagttcgacggcgagatcggaggtaagcatcagtaagcctggatactggtggtcagggtgttgacggtcaaggtccagccgtccacgagaacgaacaggagcagcttgagcggcagcgagatgacgaggggcgacagcatcatcatgcccatcgccatcagcgtcgaagcaacgaccagatcgatgatcagaaacggcaggaacaacatcgcgccgatctgaaacgccgtcttgagctcgctcatcatgaacgccgcggccttcacgacgaatgcgtgatcctccggcttcgcgacatcgcgctcgccggacagccgcgaaatctgttcgagactggccttgctggtctgcgcgagcatgaagcgcgagaccggctgttccgcacgcgcgatcgcttcgcggaacgtgatctcgtcgcgatcatagggctcgatcgcatggcgccagacgtcgagcccgacggggcgcatgacgagcagggtcaggagcagcgcgacgcccgtgatgacgcgagccggcagcccctgctgcagcccgagcgcctgccggagcagcgcgagcacgatggcgaagcgaagaaagctcgtcatcacgatcgcgagggccggcaacagcccgagcagcgtcatgaacagcaggatttgcgtcttgacgctgtaatcgccacgcaagccgagcgattcgagcggcagcttgagcggctcggccagcgcgggcgtggcggtgatcgcgctcagggcggccgcggcggtcagcgccgtcgcaagccgcgcggcccggaaccgcagccgcgtcatggcgcaaaatctccgaggttctcgagatcgatgatcttgagcccgtagttctcgccggacacgacgacttccgcgtggccgacggtcgtgccgttgaccttgacgaggagcggctcgccggccagcctgtcgagttcgagcacagtgccttcatcgatgttcatcagttccgcgagggagacggtggccgttcccacttcaagcgtgagctggaccgggatcttggccagcagttgacgcgggtcgcggcgcggtgcgtgcgccgccgttgcggtatccgccggcgcatccgccggcagatcgtccagcagtgcgtcgaggtcgttcggctcgttcgagtccatccattactccacgtagacaaaggaagtcagacaaagcttgccgttgtgctcggcgatcgacgcggtgaacaactgcgcgtcgtgggtcattacccgcgcgggcgtatgcaggcggacaggcagcacggtgccgggccgaaggcggagcacgtcgccgaaaggaacgctcatctccacgagctgtgccgtcagcttgaggcgcaactgggtcgacggcgacggacgttcccgcttcacgctccgcgcgcggctcatcgacgatttcaggtggtcgaacagacgctgctgccagactggatcgagcgcgaagcatatttcgccgactgtccgggacgcagcgccttcgatcgagcagatcgcgaacagatgggcgccgctcgacggcgccggcggctcgccggcgggcgcgtcgaaaggaatcgcgcgagcgaccgtgcgggcgagttccgccgcgaggcgctcggccagccggcgttcggtggtggtttccggaatcggcggtgcgtgcgtcgccgccgtttcggctggcgtcggtggggcggaggcggtttcgccgtcgcttgccggcggcgtgtcgccggcatcggccggagcggcggggggcatgacgggtgagatcgccgggccgtagcgcagctccagaagcagcagcagcagcgcgcgctcgatgtgcacgccgatcgccgctcggtcgactgtttcgcgccgccacggccgcgcttccagttcgggcacatgcgacgtgaacgagacgtttcgcaccgccacggcgacgccgtgcctgcggccgaggctcgtcgtgaagaactggtcgaggtattgcctgagtgtcgcgccgaacccgtccagcaagtgcgtcggtcggcccagcattctcggatccagcgcttgatgacggatagatcgactcggttttttagtcggatgattagccatcggcttcaaatcaggtttaatgatgtcgcgatatttgacgcctttgtcattgcaaggcgtcgcttcgtcacgcgaatttatggggctgaattaaaaatcaaggggacggcgagcgcgtgaatcgagttgcacatggtgattcgtcaaagccgtcgcaaattcggtttcggtctatttaaaagactggaaattggcgatcagtcgatttcccgggagatcgatgccggatagcgaagcgcatattctgccgtacccctggcgcaggccggaaatgaataagcgtgcgctgcccgcagtcgccgtgaaagatgcggaaaagccctgaattcccgcccggggaaggcttcccgggtggggtcggacgccttcttgggggcgcttgccagggttgggcggtgcgggacggttggggcagcgaaaatgcgccgggcgatggccgtgcgccggtgtctcgtatctgtccgatgaacctggccgtgcgttggaaataatccgatgcgattggcggggtgaatggcggggtgtggatcaggattatcgacaattcatgacgtttcagtgcgaatcattgagtttcactggtgacttgtattcggtcgatcgaaaatagccgtctctcaacgatttgccggccatgcatgcgattaacttgcgtgtgggagttcattgactcgcattcccattattcgcggctttttccgctgcgccggaaggatcgtatcccattcaattcatatagaaattaaacggtatggaatcgattcaacaggtgtcgtcgcaattgcgcggtcggatggcggaactcgccggccaggccgcgtctttgagcggcgcgattgtcgggcagtccggcggctccggcgaggtgaatttcacgcacatgctgggctccatgctcaaggaagtggacgaccggcaactcgacgcggcggaaaaagccgtggccgtggacagcggccgaagcgacgacgtcatcggcgcggtgctctccagccaggaggcatcgttgtcgttctcgatgctgacccaggtgcgcaacaagctgagcacggcgctcgatgatctgctgaagatgcagatctgacagcggcgccccgtttttcctacaaaggtccgctcgtgctagccaaactcaaagctgccgtcgtcagtcggcttccatcgaacctgccgtcgttgatgccttcgccggccgcgctgtcgcgactgcttccgctcgtcgtgctcgcggtcgcgttgactgccgcggcaacgctctacatgcgctacgaccaggcgaattacaagccgctgttcggtgcgcgcgagccggtgcaactcgatgacgtcgtgtcgacgctcgacgcccagcgcatcccgtaccgcatccatccggataccgggcaggtgctcgtcgcccgctcggacgtcgcgcgcgcgaggatgaccctcgccgcgcgaggcgtcaccgcgaaggccgcgccggggctcgagcagatcgatcaggacgatccgctcggcacgagccagttcgtgcaggacgtgcgattccgccggggcctcgagagcgaattggcgcaaagcatcgcgacgctcgaaccggtcgagcgcgcccgcgtgcatttatcgattgcgaagtcatcgtcgttcgtgatgatggacggcgacaagagcgccgcctccgtcgtgctgacgctcaagccgggaaggcgtcttgcgaaggagcagatcgccgcggtcatcaatctcgtcgccggcagtgtgccgaatctggcgccggcaaaggtttcggtcgtcgatcagcatggcgcgtttctgtccgcgcgcgtcgacctggacggtgatctgacgggcacggacgagatcgcggcgcggtttcgcgaagacgtgacacgcaatgtcgaggatctgttggcgccgtcgctcggcctcgggaatttccgcgtcagcgtgacggcgctcgtcaacaacgaccgtgtcgaggaaacgcgcgagcagttcggcgaagcgccgaaggtgatgaacgaggcgacccgcaacgagcagaaccgcgatcgcgtcgcgctcggcgtaccgggctccttgagcaaccgtccggtcgacgtgcccgcgtccgagccgcaggccgacggcggcacgcaacgcgccgcgacgacgcgccagttcgcatacgaccgcaacatcacgcagatcaagcgtgggcgcgggcagctcgagaagctcagcgtcgccgtcgttctcaacaacgccgtgtcgcccgccaagggcaagccctggtcgccggagcaattgcggcgaatcgacgagatcctgcgcagcggactcggcatagacaagcagcgcggcgatcaactgatcgtgtcgtcgctcgatttcgcggcgccggccgctgccgagccgtggtggaaggagcgcgacaccctcgagcagggcggctggtatctgacctacgcgctggcggccctgttcggctacctggcgctcgctcgccccatgctgcgcatgctccagcaatgggtgcagcaccgttatggcgcgccggccgccgctgcttcggcgtccaggctgccggcccccgagggcggccacgcgcaagcggcactgccggctgcggacgacgcgcggccgcccttggcggcgatgttgcctctcctcgatgacgtggagctgccgcctgccgattcgggcgtggaggtgctgatcgaccacttgcgttcgctctccgagaaggagcccgagcgcgtcgctgaagtcgtcaaacaatggatacggaatgatggaaacgccagtagctgatagcaatgcccagggctcgatcgagcaggcggcgatcatcctgctgagtatcggcgaggagcgcgcggcggacgtgctgcgctgtctgtcgcgtccggaactgctgagaatcacgcagacgatgtccagcatggcgggcgtcaaggtggacgtcgtgaaggacgcgttgcagcgcttcttcgatcagtatcgcgaacaaagcggcattcacggcgcgtcgcgcgtgtacctgcagcgcgcgctggatctggcgctcgggcgcgacatcgcgaacacagtgctgaacagcgtatacggcgacagcatccgtccgaagatggcccggcttcaatgggtgtcgccgcgctggctcgccgaccatatcgttcatgagcacgtgcgcatgcaggccgtgtttctcgcattcttgccgcccgcccaggcgagtcaggtcatcgagatgctgccgccgcacacgcgcgacacgatcctcatcaatgtcgcgaagctcgacgaagtggatcacacgctgctcgtggagctcgaggagctgatcgagctgtgtctgaaggacctggatctgcagggcacgaccgtggagggtgtgcgacaggccgccgagatcatcaaccgcttgccgggcgaccgcatgcagatgatcgagttgctgcgcgcgcacgatcctggcgtcgtcaccgagatcgagaccagcatgtatcacttcgacatgctggcgcgccagagcgacaacgcgatcggcaggatcgtcgaggtcgcgccggtcgaacagtgggcgatcgcgctcaagggcgccgatccggccgtgctcgcggcgctgcaacgggtgatgccacggcgccaggtgcaggcgttcgacgacacgattcgccgcacggggccgctgccgtccacgcgaatcgagcaggtgcggcgagacatcatggcgcgcgtcaaggcgctggccgacgagggcgagatcgagttgcgcctcgtcgaggaggacgtcgtctcgtgaaggcctatcagcgcttccggtttccgccgctcagatctgtcgcacgcgatgtgtccgcgcgcgagccggccgcgcccgccgccgcagcgcagggttcggacatcgacgcgcgcatcgaggaaggattccgtcagggcatggaagacggttatcgcgcgggattcgagcatggggaggccgaggggctcaagcagggtcgcgatgccgggtttgccgccggcgcgcagcaggcgcgcgatgcgctgcgcggcgaatacgcgggcatcgcgaaaacgctcgatgcgatgcgcgacgcgttcgcgcgcgttcaggccgagtacctcgcggcccgccgcaccgaactggtcgacgtggtcgcgaaggtggccaagcaggtgatccgctgcgagctgacgttgcagccgagccagatggcggcgctgatcgaggaaacgctggaaacgatgccggcatcgggcgaggctcccgaggttcacctgagccccgacgatcatgcgcgtctggccggcctgttgccggagcgcgcgcggcattggcggctcgttcccgacgcgcgtctcgaagcgggcgaatgtcgcgtgttgctgggcggcaacgaagccgatgccggttgtcagcatcgtctcgacgcctgcgtcgagcgcatcggcgaacaattgccgggcgctccctctgacgttgcggcgggcgcgacgatcagcgacgtgcaggaggaggcatcgtgattctgtccgcgttgcgaaatctggaactggattcggtgccggtcgcccgcatgacgggccgcctgaccggcgtgacggggctgctgctcaaagcgtccggttgccggctcgaaaccggcgagcgttgcgtgatcgaatcggcggacggagacaggatcgatgcgcaggtggtgggcttcaatcgcgaggagacctttctgatgccgttcaagaagccggtcggtctcgtcgcgggcgcgcaggtgctgtcgtgcgagcggcagggcttgctgcggatcggtcgctcgtggctcgggcggatcgtcaacggcctcggcgaggccatcgacggccgcggctcgctcggcggcgatgcggtgctcagggcgcatcccgcgccggtgaatccgctgcgcaagcgcccggtgcgcgacgtgctggacgtcggcgtgcgtgcgatcaacggcctgctgacgctcggccgagggcaacgtatcggtctgatggcgggcagcggcgtcggcaaaagcatgttgctcggcatgattacgcgcgcgacgcaggccgatgtcgtcgtcgtagggctgatcggcgagcgcggacgcgaagtgcgcgaattcgtcgaccacgcgctcggcgaagccggcctcgcgaaatctgtgctggtggccgcgccggccgatgagtcgccgctgatgcgcctcaaggcggcggagctctgccatgcgatcgccgctcattttcgcgatacgggcgcgaatgtcctgctgctcgtggattcgctgacgcgctatgcgatggcgcagcgcgagatcgcgctgtcggtcggcgagccgccggccacgcgcggctatccgccgtcggtgttcggcatgctgccgcagcttgtcgagacggcgggcaatggcgagggcgcgggcagcatgagcgcgatctacacggttctcgccgaaggagacgaccagaacgatcccgtcgtcgataccgcgcgcgcgattctcgacgggcacatcgtgctcacgcgtgagctggccgagcgcgggcactacccggcgatcgacgtgccgcgttcggtcagccggtgcatgagtcaggttgccggcgccgagcacggggcgctcgcgcgcaaggtcaaggaaatgatcggccgccacgagcagattcgcgatctgattgcgttgggcggatacgtgcagggcgctgatccggcgacggacgaggcggtggcgaaggcggcgcgcatcgacaccttcctgcgccaggacatgctcgacgtcgcgcgtcacgacgccacgctggcggcattgaaggagctgatcgcatgaacgaacaacgcaaagtcacgcgcatcgcgggctggctcgccgcgcgccaagccgaggtcgacaggctcgccgcggacaccgcgcgccaggaagccgtctgccagcgctaccggcgctacctcggcgcgatgcaacggctgtacgacggatccgccgcggcgaccgccgcggccctgccgatgcttgcgcagaaccacgccggttatcgccagacgctgctcgacaccctggcgcaactccgggccgcgttgaaggtgcaagaggacaacctggaagtcagccgcgcgctgctggccgcgaagacgctgcggtgcgaagtgtggcgtcgcgagctcgatcgcaggcggcgcgcgctcgagctcgcgcggcggcgcagcgagcagcgtgtgcaggacgaactggcggtgcaggcgtggcggcggacgccggcatgacggcatgacggcatgacggcattgacgggtgtggacggcgatcgccgtccacacgatgtatttcgagaaatggagaaaggctcatgttgagagtgacggatccgcaggcaatggcgaagactttcgccgaggcctacatggaaagcgcgaggtggcagctcgagcggcaagcgaatgacgtgaagaggaacaaggatgcgctgtcgggtctgggcagcttgctgaccacgttccgggacaagcagcgcgagcttgtcgcgggcggcagcatgatccagaacacggtgacgcaatcgaacgatgcgttcgcgtcggtagcgacgacgtccggggcgaagccgggcagctaccagttccatgtcgaacaactcgcgacggcgcatcagctgacgatcgacgtgccgtccggcattcccgccgcggacgccggctcggtgaagatcaagctggccgacggatcggaattcaccgtcgacctcacggcggccaagggtgaggatggcaatgtgtccgccgaagaactggctcgcgtaatcaactcgagttcgggggcgagcggcaacgtccatgccgcggtgatcggcggccagctcatgctgacgtcggccaatacgggggccgacagcaagatttccttcgagatcgattccacgtcgccgcttgccggcgcgtttgacggcgccgtgacgatctccgaggcgcagaacgcgaaggtcaagatcggcgacccgacgaacggaaagacgatcgaactgcagggcaacacgcttgattcgatcgaagggctgagcatcacgttcagacaggtcggatcgggaacgattacggttgcttccgacaaggaaaagacaaccgagagtgtcaaggcgtttatcgatgcgtacaacgaactgatcgacaagctcgccgagctaacaagagtcggcgatgcggcgaaaggtgatccgggcgggccgttctcgtccgacgcgggggtgcgcgcactcgtcagccagctcagatcggagttgaacaagagcgtggatggcgtgaagttgtacgagctcggtatcgatacggacaagaagggcaagctggtcctggatgaaaaacgtttcgagcaggcgttcaaagcgtcgccggatcgcttcgacaagctgttcggcgatgcgcagaccgggctcgtcggcaatctcggcaaggcgctcgacggctggctgaacgaaaagaacggccaactgaagacacgcaacgaatcggtcgcgcaggcggaaaagcagcttctcgccaaacagcaagagtacgaccgccgctaccaggacgtgttcgaacgctatacgcaggaattcacgcggcttcaggtgctccagctgcaaatggacaacacgctcgaccagatcaagaacctgttctcgacgagcaaggattgacgtcgcgatcgcagcttcgaggaatgaaccaacgtggaaaacatcgggtaccgcagctatcacgctgccaatcttgaatcgcagatcgcgaacgcctcgccgatccagctcgtgctggtgctgaccgacggtctgctggacgaaatcgcgcgcgcccgcgcgcacatcgtctcacggcgcatcgaggcgaaaggcgcgagcatcggccgatgcatcgagctgctcaacggattggccagttcgctcgacatgacggcgggcagcgaggtggtgacgaacctggcggcgctttacgactactgcgtgcgccgcctgaacgttgccggcatcgaactgaacgtcgagccgctcgacgaagcggcgcatctgatcggggtgcttcgcgaagggtggcagggtgcgcaagatggcggactttgatgttgatcgggtgggcgacttgtccgctgcgttgacgacgcgcgcgcgccggctcgccgacgccgcgatgcagcgcgactgggcgtcggtgacggcgctggatgcggagctgaacgcgatcgtgcgccgctgcgccgcggacggcgagccgacgcgggacatgcatgccgcgttgcaggccgtcgcgcgcgtgtacgaacgggccgccgcgacgatcggcgacgcgcacggcgaactggcgagcaggcttgtcgagctggccgagaaccggcacgcatggatcgcctacgcgacgatcgccgagggcgggcaatgaacgcgatcaccgcgatgacgggaccggcgaccgatgcgagcgggcgcgccggtgcggcttgcaccgtgctgctcggcgctggcacggtgcgtggacggccggccggcgcatcggacgacgaggcccggacgcagggcgccgaatcgccgccggagacggatcgtttggaggatgtttcgcaatccggcgcattcgccggggtgatcgcggcgctcgaggttcaagcgcccgttcgggatgtgcaggccacggacgattcggcgagagacagcgaggatgccgcgcgtgagcgcgttgcggcggccgcggcagatgcagtgacggtgggtgggaccgtccgtgcgttgcgagatgggctcggcggactcgcgtttggacagagtccgatgatcgaggcgggtaaggcgctgcgcgtcccgccggcgcgcgatggcgtacggctgcccaatgaagacccgctgcgcgacggcgaacggctccgcgacgagactggcggtgcgcgcgctggcatgcgcgattcggtgtcggccgccggcgcggcatcggcggcccgtgtcgggcagccggccgcgggcgcgccgcgcgatcagcaagccctgtcgcaggtgcccggtcaaccggccgccgacaagccggcggccggtgcgcctgggccgcaggacggtgcgatcggtgcgatcggtgcgcccgttgacgtaccgcgcgcggccgctcgtgcgtattcgccgcagtccgcgcccgtgcgcctgggagccgagctgacgcgcgtactgggcgagcgcatcgatgtgcagcttcaacgcggggtcgagcgcgcggtgatccggctggaccctcaatcgatgggcacgcttcatatcgcgattcgtcatgagggaggaaccgtccagatccagatgatcgcttcgcatgaagaggtcgcgcgccaattgcagacgatgagcgaggcgctgcgccaggatctgtcgtccaggcatcacggcgatgtcaccgtggtcgtgaggcatggccagggcatggaacaaggcgcgcgccatggcgatggcggcgcgcaccgcgaagagaagcggcccggcccggcgcttgccgaggctgacgtgccggacggcgacgaagggtttcacctcggcggacgagacgtgatttaacgatcaagtaggaaacagattgcgatgaaaaagaagaccgttgtgtcgatgatcgtcggcgcgacgttactggttggcggcgccatcggcggtacctgggcgtttctgagccgcaacgcgagccatcgtcccgcgccggtcgcggcgcaggacaagtacaagtacgtcggggaaagccagattgtcgtgatgttgcgacagcaggacggccagaacgccacgtcgatcgatgcgcaggcgcactacgcgatgctcgacctcgtgctgaaaaccacgcccgaaaaggaagccattgtccgcgatcacatgttgctgctgcgcagcctgacggtcgacgcggtgtcgcagtacacgctcgagcagctcaggcagatgaacgtcgcggagctggcgaacgtgctcgaccgcgcgttccggcagtcgtacaagacgcgggccgtcgcgatgccgttcgagagcgtgatgatcggcaagcttctcgccgaatgactgcaatgacgacactggacgattgcgccgccgcgcgcactgcttatgcgcgcgccggagcgggcgccgagcagagatggctggtggagtacgcaccgatcgtcaagcgcgccgcccgccagttgaagtcgcaggcagtcggcatgctgacgctcgatgacatcgagcagattggcctgatggggctgctcgaggcgctgcgccgctatggtatgccagatgcggccttcccatgctacgcggcgactcgcgtgcgcggcgcgattctcgacgagttgcgccgtcacgactggcggccgcgttcggtgcgcctggaggcgcatcgcacgcgcgactgcgaacgcgagctgtgccgctcgctcggacgcgagccgacggaagccgaggtcagcgatgcgctcggcatcagcacgacggaataccgcgaacgcgtgatggcgaacaatgcggaggccatggcgagcttcgacagcgtgctcgacgacgacgcgttgacccacgaagaaagccccgaaagggagatcgtccggcgtcgctgcctcgagcaggccctcgctcgcctcaacgagcgcgagcagcgcgtgatccagctgtactacgaattcgatttgagcctgcgcgagatcgcgtccgtcctcggcttgaccgaggcgcgggtttgccagatcaacaaagcggccctcaagaagatgcgagaggtgttgagcgatgcatgagcgcgccaacgcgtcagatgcgggaatccgtcctttcggagaacgaacatgagattgttcagcacgaaatatggcgtgccggcgcaacgaagcaacggcgcgagccgtgcttcgacgacaacgcacgaaggcggcgcgcgcggcgcggcggcgggcgatgcgggtttggaggtcgagctgttgcggccggcccgggaggtgctgcgcgagctgcccgatttcgacgcggcgcgcgtggcggcgttgcgcgccgcattgcaggaagggcggctgccgttcgacgccggcaagctggccggactgatccagcgttttcacggagcgaaggaatgacgcgcaaggaagcattctcgcgaatgctcgccgggctcgaggcggaccggagcagatgcgaggagttgaggggactgctcgagcagcagttcgatgcggcgctgcgtcgcgacggttcgttgctaatggcggcgggcagcgccattcgggcgctggtgcaagcgctcggcacgagccacgaggcgcgccggcaatgcgcgcgcgtgctcgccgcgccgaagcggcccgcgtcgatgagcgaactgtgcgcattgctgcggggcgcggcccgcgatgcgctgacgctcgaatggcagtctttcgatgcgctgatgtgcgagtgccggcgactgaatcagcgcaacgccacggtgctgatgtctcagttcgagatcgtgcagcgtgtgcttcacggcgagagcgatacctatgttcatccctgaattcgatggccgttcttccgattgggcgcgcgcggcgtcagccggcccgggcgtcgcctcatggagcgcggcgcgaggcgagtttcgcgcggaattctcacgccttcagcgggatatcgccgcgacgatcgcgcaaggtttcgacacatccggcgacggcggccgcgttcgcattcagggttccgcgccgtttgccggggcgaacgcgatcgtacgtttgctggacgggggatcggcgcatgcggcgccggagaaggcggcatttatcgagcgcatcctgccgtacgcgaacgaggccggaaaggccttgtccgtttccggcgaactggtggcggcgcacgcggcgctcgagtcgggctgggggcggcgaccgttgacgacgggcgaaggcgcgaatacccacaacctgttcggcatcaaggccggcgcgaattggacgggccacgtcgcggacgtgctgaccaccgagtacgtcggcggcgagcccatcaagacggtggagcggtttcgcgcgtacccggattaccggtcggccttcgcggattacgcggcgctgttgcgcggcagttcgcgttttcgcgaggttatcgggacgggcgacgacgcggccgcgttcgccgccgcgctcgctcgcggcggctatgcgaccgatccggcttattcgtcgaagctgcgccggatcgcggccgacgtggcggcgtcgctcgccgccgccggcggtatcagcgagacgcgtccggcgctggctgggcaatcggctcggccgtccccggaccggtgacgcgaaccgcaacgactttcgcgtccctgcgcgtgcccacgcgaaggcggatgacgtcgtcccgcgcgccgttttgcatcgcgatgccggtgttgacgatctcggcctgaccgacgcgagcaacgatccgcaccgtttggccgcgacggatcagctcgggcgcctggagaatgcggcgcgacagcacctgccccgcatgcagcgaacggcggctcgttcggccgatcgcctcgtcggcgcgggtcacggtgtcggtcagcgaagtcagtatgcgacgcgccgtgccgagatcgtccgccgcgatgggctcgcgggcatcgaggtcgcgcgcggcaacgggaatcgttgcggtgacctgggcccgggccgtgagcttcgtcacgcggccggatgcggggcagcgcgccgacagggcgagcctgccgagctgacgcggttcgccgacactgacttcgaacggcgtatcgcagacgggcgcgggcggcttgagcggcgaaacgacgaccgcgatatcggcgtcgcgcagtccggcgcgcttcgcgagttccgcgaggcgggtcagcgcgatttgccgagcctgctcgcgaagctcggggcgcggcgcggcggatgcggccggcgcggatacggccgccgtggacacggccgacgcggacgtggctgacagagaaagcagcaacagagacgagagagagaagacgcgcatagcggaccagtgggagaaacgggccggccggtgacgcaagtgccgcatcgtgatacgcggcgggtggtcacggccggcagtcgaaattgagcgaagtgtatagcaaggaacacgatatggcatttgatctggcgagcgggctgggccttcatcccaccgcgttgaagctgcgggcggaacgaacgaaggtcatcgcgtcgaatttggcgaatctcgatacgcccggctatcgggcgcgggatctggatttcaagcgcagcctggccgaggccggcggcgacggcgcgcacgcgcgtcccggcgacgcgctgacgggcggcgtgacgctcggctatcgcgtgccgtatcaggcggcacaggatggcaatacggttgaactgggcatcgagcaggcggcgttcgcgcagaacgcggtcgactttcagatgagtctcacgttcctgaacacgaagatcaagggcctgcaaaccgcgattacgggcaatgcgtaagcgcgttttttcttcaaacctgtttatcggatcaccatgagcttcaaggacatcgcacagatagccggttccgccatgtcggcgcagacggtacggctcaatacgatcgccagtaatctggccaatgcggattccgcgtccggcaccgaggacgcgacctaccgcgcacgcaagccggtcttcgcgtcgatctttcagctcgacgcgcagggcgagccgcgaggcgccgccgtgcaggtgctcgacgtcgtgaagagcaccgagccgctgcgcaaggttcaccaaccggggcatccgcttgccgacgccgagggcaacgtgctgtatccgaatgtgaatccggttgaggaaatgacggacatgctgtcggcgtcgcgtgcgtattcgacgaacgtcgaagtgctcgggcgcgtcaacggcatgcagctcgaactgctcaagctcggtcagtgaccgcgcgcgaccgatacgaggacaacatgtcgaatacgatccagccccgcgcgggcgatatgcagcaaccaggcgccggacagcacacgccgtcgccatacgccaacggcgcgaattcgccgacggatctgttcgcgaagctgctcgctgcgcaaatccagaaccagaatccgctcgaaccgatggacgcgtcccagttcgtcacgcaaatcatgatggggcagcagaccgaggcgctcggcgtcgtcgcgaacctcacgcagaacagcgcggcgctgatggaaagcatgctcgtcgtttcgctcgggtcgcaggtgggcacgcaggtgatggtgcgaaccgattcgctcgagctgaaagacgacacgcgcgacgcccgaatcgtgctcgataacgccgaatcggacgtgaccgtcgtgctgaccgattcagccggcaattcgcatcggatcgcgcttggcgcgcaagccaagggcgatgtgaatttcacgatcgatccggaggagctcggtttgcccgccggccagtacaagattcgcgtcgagaccgccagcggcgcgaaagcgggcgtcgagatcgaagggacgctcgagaacgtccgcctcgcggcggacggcaaggtgattcttcatgtcgccggcgtcggcgatgtggagacgtcgtcaatcagcagctttctgggtcgtcgacccgccaacgagacgcgcaaggaaaccgtatgagcttcaatatcgccctatcgggcatcaacgccatcaacagccaactgaacacgatcagccacaacatcgcgaacgccaacacctacggcttcaaggccgggcgcgcgaacttcgcgacaatggtcgcgggcacgcaggccaacggcacgtacatcggatcgatcacgcagagcgtcggcacgcgcggcggctttctgccgaccggccgcgcgctggacggcgcgatcgacggcaacggcttcttcgtgacgaaggatagcgacggctcgatgctcttcacgcgcttcggctacttccagcgcgacgcggacggctacatcgtcgacggcttcggcaggcgcgcgcagggttacggcgcgagcggcgcctatggcgacattcgcgtgccgaccgacacggagccggcgaaggcgagcgacagcctcgaatatgtgggcaatctgtccgccgactgggcggcgccgaagaacccgaatttcgataaggacgacgacacttccttcaatcattcggtcgcttcgaccgtctacgactcgctcggtcgtcagcacatcgtgacgcagtacttcgtgaaggggcagccgccgtcgaccgacgtcatcgcctattacgcgatggacggcgagatcgtcggcggcaatacgccggtccagacagtgttgcagttcgacacgaacggccaactgaccgcgccgaactcgccggtggatctcgatctcggtacgcccgccggcgcgtccgcgctcgcgatcaaggtcaactacgccggcacgacgcaagtcgccggcgagacgacgacgacggtcaatcgcgacaatggctatgcagcaggcgtgccgggagaggtgatgctcgacgaggacggcggcgtcgtcgtgcagtacagcaacggcaagcagcgcaaggtcggctcgctcgcgctcgcgaccttcgcgaaccaggatggcctgagcgccgtgggcgacaccgcctggcgcgcgtcggccgcgtccggcaatccgttgatcggctcggccggctccggcgcgctcggcaaggtggtggccggttcgctcgagctgtcgaacgcggacgtcacgcaggagctcgtcgacatgatgagcgcgcagcgcaactaccaggccaactcgaaggtgctgtcgaccgagaaccagatgatgcaggcgttgatgcaggcgctgtaaccgatggacgcactgatctataccgtcatgagcggcgcggaccgcaccctgcatgggatcgcggtgcacgcgaacaacatggcgaacgcgcagaccgacggctttctggccgaactcgaaatggctgaatcgcaaccggtgcgcggctacggctatgacagccgtcacctgctgaatccccgggcgaacgcgctgaacggcgaggcgggcgtggtcgtggataccggccgcgagctggacgctgcgatcaatggcggcggctatttcacggtcgatgcgcagggcagcgaggcgtacacgcgcgcgggcaacttcgaaatcgacgccgacggcgcgcttaccgtcaacggccggccggtgatcggcgaaggcgggccgatcgtgctgccggaattcaccagcctgcgcatcggcgaagacggcacgatttccatcacgtcgccgggcgacaccgaggtgcaggcggtcgagcgcctcgcgctcgtcgacgcacccgcatcggcgctcgtgaagaacgaggcggggctgctcgtcgcgcgcgacggccagccgttgccggcgtcgaacgaggtcaaggtgctgggcgggcatctcgaacgcagcaacgtgtcgcccgtcgaggaaatgatcgcgacgatgtcgctcaatcgcgcgttcgaggtgcagatgcggatgttcaatgcggcgggcgagatggccgacgcgggcaaccggttgatccggggctgacggccggcgatcaaacgaacaggagagtcacatgaatcaagcaatgtggatcagcaagacggggcttcaggctcaggacgcgaagctgcaggcagtcgccaataacctggcgaacgtgaacacggtcggcttcaagcgcgaccgggtggtgttcgaagaccttttctatcgcgtcgagcggcagccgggcgcgcagctggatcaggacaacgcgtcgccgtccggcattcagatgggcagcggcacgcgcatcgtcggcacgcagaagatcttcacggacggcagtgtccagacgaccgggcagtcgctcgacgtcgcggtgatgggcaagggtttcatccaggtcctgatggccaacggcgagaccggctacacgcgcgccggccaattgagcatcaacgcggaagggctgttggtgaatgcgcagggcctgcccctgcagccggagatctcgattccggagaacgcggccgacatcgcgatcggcgaaaacggcatcgtcgccgcgacgatgcccggcgcatcgtcgccgagcgaggtcggtcagatccagcttgcgaacttcgtcaatccggccggcctgctggcgctcggcgacaacctgtacatggagacagtggcgagcggcccgcccaacgagggcgagccgggcacggacgtgttcggcaagatcaagcagggcgcgctcgaagggtcgaatgtgcaggtcgtcgaggaaatggtcgacatgatcgcggcgcagcggacctacgagatgaacacgaaggtgctgagcgcggcggacaatatgctgcagcaactgtcgcaggcggcgcgatgatgcgcatcctcctcgcgttgacggggcttgcttgcctgggggcctgcgcgtcgatcgagccgccgcgtccggatttcgcggacgacgaccttccgccgatcgcgaccgcgcccgccgccggacgctccggcggcgtgttcaacgcggtcaccgcctggtcgctggcgtcggacggccgcgcctatcggccgggcgacacgctgacggtgacactcgaggaaaccacgcaggcgagcaagcgggccgacaccaagttcggcaagaacggcgacatggccgtcaagccgggcgtgttgttcggcagtgcgatcccgttcgattcgtcgttcggcgcgaaacgcaatttcgacggcggcggcagctcgtcgcagcagaacacgctgcgcggcgaaatcaccgtgatcgtcaatcaggtactggcgggcggacttctccaggtgaagggggaaaaggtgttgtcgctgaatcagggcgaggaggtgatgcgcttgagcggttacgtgcgccaggccgatatcgataccaataaccgcgtgtcgtcgcgccgcctcgcgaatgcacgcatcaaatacgtcggcaagggcgcgttgtccgattcgaactcggcgggctggctgacgcgcttcttcaacagcccatggatgccattctgatgaaattgagatttcgagatcattattcgagaaggcgcacgcgggctcagcgtgtgtgtgcgttcgcggcgatcctgtcgctgctgtccgtgctgctcatggcggcgtcgcgcagcagcgacgcggcgccgctcggcacgctggtgagcgtcgagggtgtgcgcgacaaccagctcgtcggctacggcctcgtcgtcggtctgaacggcagcggcgacggccagcagatccggtacacggggcaatcgatcgcgaacgtgctcaagcagttcggcgtcacgctccccgaaggcatccggttgcgctcgcgcaacgtcgcggccgtgatggtcagcgcgaatttcccggcgggctacgtgcccgggcagaagatcgacgtgacagtgtcgtcgatgggcgatgcgaagagtctgcgcggcggcaccttgctgctgacgccgctgcgcgcggccgacggcgtggtctacgcgctcgcgcagggcaacctggtggtgcccggcgtaagcgcgcaggggcgcagcggatcgagcgtcaccatcaacgcgaccgcggcggggcggattccgcagggcgcgacgatcgaacaggagatccccagcgacatcgacgcaaagccgttcgtacggctgagcctcaagcgcccgagttttcagacggcgaccagcatcgtcgccgcgatcgaccgcatggcggggcctggcgcggcgacgagccgcgacggcacgagcgtcgaggtcagggctcccgaagatcccaccgcacgggtcgcgttcctcgcgaagctgaccgcgatcaacgtgacgccgcagaaggagccgccgcgcgtcgtgttcaattcgcgcacgggcacggtggtgatcagccagggcatgaccgtctcgcccgccgcggtgtcgcacggcacgctgaaggtcacgatttccgaaggagcgatcgtcagccagccgaatccgctgggcggcggcaagacggccgtcgtgccgctttcgcaagtggacgtccagcaggacggcaaccgtatgttcaattggcccgcgggcgtgagcctgcagaagatcgtcgacacgatcaattcgaccggcgcgagcccggacgacgtgatggcgatcctgcaggcgctcgacgaagccggtgcgctgaatggcgagctggtcgtcatctgacgaggagaatgtgatgtcgaatggaacgatcggcgcaggcgtgcggatggcgccggcggcgcggatgccggggctcgcggcgcaaccggccgcgaacgaaagcgatgagcgaaaggccgcgtatgcggccgagcagttcgaggcgctgttcgcgtcgcagttgctgcgcgagatgcgcaatgcgattcgcgagatcagccccgagggcagcatgtttcgggatgcggtcggcagcggcctgatggacatgatggacggcgcgatcgcacaggcgctcgcgacccagcgcgcgttcgggatcgcggatttcgtcctcgcccggattatgcccggcggcgatccgaccggcgccgccgcgcgctgacgcggcgcggatttaagtgacgcgcgttccgggtcgccaataccgagtgctaccttcccgtcagctccgcggccggccgcatgtcgcggccgggtgcgcgggcatagaaaaggcttgtactgcgatgaacatcatttatatcggaatgtccggcattcgcgccgcgaaccagtcgctgctcgcggccagccagaacacggcgaacatcgcgaccggcggctattcgcggcgcggcgtgttgttgtccacggggctcacgggcggcgtcgacgcatcgggcctgctgcgcctgaacgaaagctacaagacccagcagctctggcaggcggctggccagtacggccggtacgacgccgcgcagacctatttcgatcagctcgaggcgctgttcggcggcaatcctgttgacggaaaaacccaggcaaacgatctcggcttcggcaagttcatcggcgcgctggaggcggcgagcggcgatcccggcgattcggtgctgcgcaacggcgtgattgacgcggcgaattcgatgacgaagcagttcaacaacatgcgctcggtgcttaccaatcagcttcgctcgatcgagcagcagcgcgtcacgacggtcgagcaggtcaacggtctcgcgaagacggtcgccgagctgaacgggaagatcgcggccggacaggcgagcggcgcggacgtttccgcgttgatggatcagcgcgacgccgctgtcgacgacctgtcgaggttcgcgggcgtacgcgtcgtgcagcaggcgaacggctcggtcgacgtgtcgatggcgaacggccagccgctcgtggtgggcgcccaggccggcaagatcgaactgggccgcgacgcggccgggcaacagacgctttcgctcacgttcggcagtcaggtgatgccgttgcgcgcggcaagcctcggtggcgagctgctcgggctggcggaattcgaggaagaggtcctgcgcccgcagatggaggcgctgcgcacgctggccgttcgcacggccgagctgatcaacgatcaactgacgcagggtttcgatatgaacggcaacccgggcaagccgctgctcacgtatgacgccgcgaccggcctgctgggagtcgacccgagcgtgaagcccgaggagctgggcttttccggcgatccggccaatccgggcgataactcgaacctgctggcgctcgtcgagatcggctcgacgtcggtcgatttgcccggcatcggcgacgtgtcgctcggaaacgcgtttgcgacgatgatgggcaaggtcggcgcggcgagccagcagaacaagcaggcgaaaggcgcggccgaggcggtgcgcgggcaggctgaattggactggtattcggtcagcggcgtcgaccgggacgaagaagtgatgaacatgctccgcttcaaggacatgtacaacgccaacatgaaggtgatcgcggtcgccagcgagcttttcgaaaccacgctgaattctctctgacgcaaggagtacgcgacatgcgcatcacgagcaatcaatatcattcgatcatgacgcaggtgaaccgccaggcgactgcgggcctcatggacacgcagatccgcatggcgaccgaaaagcgcatcctgcgaccgtcggacgatcccaccggcgcagtgcgcctcgcgatgctggtgcgggccgacgcgatgctggaccagtatcgcgcgacgggggcgatgctgaacagccgcctgcaacacagcgagcgtgtgatgtcgggcatcgtcgatcagctccagaacgaggcggtgaagcacctgggcttcgcgatggacggcagccgttcgcccgaggatctgagcgcatatgcccaagtactcgcgtcggtgcgcgacacgctgctgcaatcggccaatacgcgggacagttcgggcaactacttgttttcgggcacggcgattacgaccgcgccgattcgattcgatccggccgcgcccgtgggcagtcgatatacgttcgaaggcaacctggagaagcagacggccgtggtcggagacggcgtgacggaggtcgcgaacgagagcctcgcggaaatggcggacgggctcaatgcgctcgacgaagcgatcgcgaagatgagcgagccgaatgcgaatccgaacgatccggcctatcgcgacgtgctcggacgcgccacggacacgatcgaatcggtcctcgcgtccgtcaacgcgaagatcggacggatgggcagcacgcaggcgagattgcagatgctcgacgaactgcacgaatcggcccagatcgtcgggaatcaggcggcgctcgatgtcgggaagatcagccccgaggaagtctttacggattaccagagctacatggtcgcgttgcaggcgagtcagaagctctatgcggaggtcacgcagttgacgctcttgaacgtgctctgacggcggccggacgggatggaactgaaaacgacgatgcctgccggcggcgcgcgcgctgccgcggaggccgggctgcgcggcgggatcaggacgcccgccgcgatgcagcccgacatacgggatgggcgcgtgcgcgatgcatcgcacgcggttgtgcaaatgccgccggccggacgaggtgcggccgaccgccgggacgcgttcgcggtgcatcagcgtgtgaacgagcaggcatcggggatgcaactggtgctggactatctcgaacagttcggcgatcgcatgaaggctctgaagcaggcgctcagcgcacgtctggcgggcggcggcagcgatctcgacgtgcgcgcgaagatcgacagcgccacggccgcatggaaccggcgcgccgccgcgacgggtgggcgcctgagcgagacgctcgagctgggcggcgtggattcggcgcggcgcgcgttcgtcttgcgcggtctcgatgcgcaggacgcgcggcgggccgagggcgaggtcatcacgttccatctcggcggtaccggcgcacgcccggcaacgctgtatcttgcgccggatctgacggcgcgcgagatggcgtatcggctcaaggtcggccttgcgccgctcggcatcgattgcgaaacgcatgcacgggagcgcatcacgctttccgtcgaggaatcgcgctggccggtcgtcgcgcgcggactgcacgtaaagggcggcggcgtgcgctttcccgccggtgcgccgcagcgtgccgcgatggaagccgttgcgggccgcatcgacgcatcggactgggaggcgggcgatgccggcgcgatgcgggccgcgttgacacgtacggtgcagacggtgaagcggctcgatcaggtgtccattctcgccgagcacgcgctgaagcgggcgcggcagacgctggcgtcgatgccgtccggattgaatgcgcagtgggctcgggatgcgtgcgagcgcatcgccgcggacgcatccgcgcgcggcggctacggccaggttgtcgcgctgacggcggtgacggcgggtctcgatcgcgcgcgcgtgcgcgcgctcctgtcgatgcgttgagcatcgtgccgccaagcccggccgcgtgccgtgcatgcacgggcgcccgggcggcggccgtcgatggagcgcgcagccggttcgccgcgcctgtgtgcagtcgggtgggggaaggatttgaagggagaacgaaatgccggcgtcccgtcaggcgccgcaccgagcgcggcgccgtttcgccgcgtccgtcgcgcggccgcgctgccggtgtggcgcgcgtatcgtctgcggtgcacgggccgaaaatcgcctgcgccttgcacggccgaagcgcggagggaacgccagcacatgctggtttgcgttcgccatgtagtcgatacgttgaggcgctttgaagccgcgctcgcttgcgtgcgccgatcgcatagatcaagcgtgctcgaatcgatctgctcgccggacacggcgaagccagccggcgccgaagtggacgatcccgtcgcgctcgcgaatgccttgatgggcatccgccgcaggcgggtgcgcgaattgttggcgctgcgcttgccgtaaagcgggttgtcctggcccggcagggcgagccttcgaaaatcgtcaggacgtcgtcagatcccgggtggagtaggtggtgcagcgttcatcgctgctatgtcgggttcccgcgcatatcgcgagccatctgtcgccgttacgggatatctcatagacgcgctctccggggccgggccgtacgccgatcgacgcaagcagcttgagcccggcggagcgcggggtgacgtgagcgtgggtatggacgccgaaatgcccggtcagccacggaaagcgatcgttgatcgaaatcatcttccaggtgacgaggccgacccagaccacgagcccggcgagcagcgcgcgctccacgaaacgaagcctgcgcctccagttccgcgcgcgaccgacgggcggcgcggattgcggctcatgctgcgtgaggagcgcgagagcggcttcgtctgccggctcgccttgatcggcaagggccggctgggtgacgggcgtggccgcatcggtcaatgcctgcgcggaacccaggaacctcaatccttgacgaggcagcgtcttgatgagcgcgccgctgatgccgtgttcctcgaggcggttgcgcaccgagcgcacgagctggtgatagctgctgtcggtaacgacgaaccccttgctgccccatacccgctcaatgacggcggccttggtggctatcccgtcgagcaccatttgaagcagttccgattcgttggtggtgagttccgtggtttggtccagatgcgccagcgtcatcagatgcgcatcgaaggtgacgagatcttctatcaggtatttcttcattgtgtttcagtacgcattcgcttgcgacgccgatcctctggcgccgcccggagcggaaaaagtctgattgttggaaaattgctcgcgcggttcgatcgatcccgacgaggaaggcgcggcgcggtgccgcgcgattgctgaatatcgctgaatgtggtctgcaacccgatgtgcggacccaacgacacaattctgcgatagatgggaccgttcccaaatcggacatgtcctaaaaattcctttttcgccccggtgcatgccagcgcgatcaaggttcccgcttcgaattttcacgcatggcgccggcgagcgaggaggcggaacacttcagggcggatgcgggccggctccgtggaattgcgcgttcgtgcgcgtcgttccgtgtgatggcaggcgcgccggcagggccgggttgcgcttcgggcgtgtgcggcggttgcgcgattgagcctgagtgcggttcgggcggcgtgtcgcgcaagcgtcgcgtgggcgagcggatgataggaaaacgcacgccgggcatcatgcgccgcgccgcctcgacgtctcccgcacggctatcccgttgctcgctccggctcggcattttgggactcgtctcatgttttggcgcagggcgctttctagaatcagtgaaggctcccggccgaacggccccccggttccggatcgcgaaatgcagaaaacagatcgccgcggcgagcgccgcgcgaggcgtgagcgctctcgcaggcattcgcgcgaagcgtcggcgtcgggagggttccggaatcgcgcgaaagcgtatcggatgacgacgttcgacggatgcaacgatggagaatactttggcccagcttggggaggcgcgatgaatactttagaagatatggtgaacgattattcgaatttgaccggcgaggaacagggtatcttcatggccgctttgagcgattatgttcgggcatcgccggcgcggagaacggagatggtcgccatgtggcgtcgggcgactcaggagggctcttcgcaggcattgccggctggccgctcggaatgagcgtaatcgtaaatgcgaaagtatcgggaaaatggatctcgataatcggcatggtaattgacaattcatgcctgttcaggccgataacggctggtgtaacgattggcgcgtgaacgacatcgcatgcgcgcgtttttcattatcttgcgtggcgtagcgatcccgttattcgattccctccttcatattgccgctgcgtcgcccagggagcattgcgatttccgttggaatgacgccgctgcatatcggggcgcgacgctatttccgcttcttgctccgacccgttattgaatccggcggcaacgcggcatcgatgcgcgcattcgaagcaaattcgccccccggcttcttgccgaactcgacgattcgttaaagatcgtcaaatttgcgcgctatcaattaagcgaatgctgattcattgcgtcaacttgccgcaggaaaggcttctgctgagcttgggatgattgcgtttgaagggccgcgcattccttttcccgacttgtgtctgcgatcaacttcccgcatttctgattgtgcggaatgcattgctttgtcaaggaaatttaataaattcgatctcgatttgcgcaacaaacccgaactgttaaatattgtttgttaatgagatggggaattatttaatttgtatgatgtcaatgaat

SEQ ID NO: 116 - DNA, 5458 bp, Burkholderia pseudomallei
tcacccgcatgttccttctgcctcttcataaggctgatttggatcgaaccgtgcgggtgcctcgtaggtcacgcggcactcgctacgattgacgcccgacaacctgatcggcctaccgatgtcgtcgtcattcaggatgaagttcccatctccgatgatcgtgccgaggaactggccttgctcatcaagcacggtggtgccgaccggtgcggtggcaccggtcgaatctcgtatcgtcaacatgagctgtcgcgtttcggctgcatggatcgtgcgcgtcagcacgctgccccgtcggagcccgatgtcgacggtggtcgtgtcgagacgcacattcatgggaaggctttgcgtatcgagtctgagttgcgctttcgtgtagggcgtcagggaagagatgactgcggtgccgaatgggttggtgatggacgttccaccaggtgcgctgatgcgcaaacccttttggttcgggaccgacacgatggcgaacgtttccccgatccggctggatgccgtgccgaatagtccattggcgtaggcgatgcctccgcttgcagacaggtaggtagaccgtgagttggacgtactctgagccacgccggcgccaaattgagcgaagcccgtcctcatgctgacggacgcgttcgcggtctggccgctcttgctgcccgatgcgccgatggaatacgatccgtctcgtcccaatcgattgccgtaggacgtgccgaacgtgagatccccccgctgcgagcgcgtggttcgcgcactcaacgaaccaccgcctagcggaatcgacacgttggcgtagacgctgctgccgctcttggggctgatttgtgcggcaaggctgacgtttgcccgaccgatcttgcgaccgatgctcagcgtgtgagatacggagttcggtgcgccgacgaaactgttgtacgacaggccatacgtaaagctaccccaacgcggatgggcccagttgatcgacgcgttcgccgaatgcttggtttgaccggttccggttgcatcgccaagcgatccgagcgcgacatctggcgtggtatagcgaatcgagcgcgacagccatgaggcggatgcgaacacgtttcctccgattcgggacgagccttgtaactcgaactgatatccttggcccgcgccagccatccgcgaatagcgcactccgcttgcaagccatgctccggatttcgccgaaagcgttccttggacgctcgcatgaagatagccggaggccgcgagcgcggcggtcgacacgcgtagattgtcgagcggtgagaaagagatttcaccggtggcgaatatcgcgggtgcacgcgttcgatcggaattgcctgcatcctgatagcggccgatggcggcctgccaagtcgtcggctgcttcgcgtcggcatagcccatgccgacgggcatctcgaagcgtcgctttcgtccgtcctgttcggtgatttcgatttggaggtcggcgttctgcgcgacgctgccgatgtcggacagcatgaaaggcccgggcgggacgatcgtgcgatagacgacacggccgcgctgacgcacttcgaccgtcgcgttgctttccgcgatgccttggatgggaacggtcagttgcccgctattttgcaagcggtccgaactgatctggaagccgagcatcggcaggccgcccagattttcgcctgtggacgtgaactggccgatctgcgtgatggccccccacgcgtcgacggtacgcgcgaggtaggtgtcttgcaactgatacgagaatcccttgcctgaatgggtcagcgctccacggttacggaaaaaccagtttccgatattgaagccgggctcgagccgtgcctgtaagtaatggctgctgccgaaccgggtatcgaagcgctgcgcgaagacatcgtaattcagtaacaccgcctcgccgccggagtgcagtccgttgccgcgtcgccgtgggtcgaatgcttcctccggcacggtcatgtcgattcgaaactgcccagggtggagtttcatgtgaaattccggccacatttgttcgggatcggcgcagccggccagatcggatggtttgcgcagtcgcagcgtgtccaacagcgcggcgtcgaagcacggatcgccgtcgtcatcaaattgaacgttcgtcgtgtaggtctggttggcgttgagtgcgatcgtgacctcctgcttgccggggagaaagcgcgaatcgccgctgaagaattttgcaatgtcggcggaatagcctcgcgcaatcagcgtttgggtgtcgaattcaaccgccgcgctgggcgaaggcgacgagacggcgagtgcgtcgttgcttccccatgcgatgccgaggagcaagctaaacgacaggaccaaccgtcttgcgtacacaatgtatctcccggcagcggcgctggttgagttggcgagggaagcgggccgcgttgagtcgcgagctttccatgggcggaaggcgcaaccggcgatgtgcgtcggagggaggcggcgtgagcctgcatcggcacatgagccgcgtgcccggcggcaaaacgaagcctggtcaatagcgggcatcgctcgaaccaggatgttcggctggcgatgagcccgcgcggatggctattgacgtgagcgagcgccgtgccgcgggctaaattgcccgtggcgcgcatgctgcgtattgaggcgaatggcgcgcgctggcagaacggaagtcggccggttgacgtcgccgacttccgttccgccaaattaatgctggaccggcatcgtgactgcgggcagtacgaggccgtagcggctgagtggaacgatctcgattttgtcggtggcgtcgtcgatttcgacagtccgtgattcgcccggcatcagataagcatgcgcaagcgcgaccatcttgttcgtgggcagcagtttgacgctcggcgcaagccggatcacgtgcgcgctcggattgctgagagtcaattgcttatccttgacgctgagcttcaagttatcccaaggggttttggaaacgggaatcgcgccggcctggaccaaaaagccgatttcctggcgaaccggcatcacgagcgagccgcggccagtttgcgataccccttcgaacgacgccttgagcatgtactcgtgctcgagcttggctcccttcttgagcgaaaaattgatctgctgactttgaccaggatcgatacgtgcgattggcggcgcaatcaggaagcgttgactgagcttgtcttcaccgaggtcttcgagcttgctgatgagcagcatcgggctttcgccgtcgtttttgacgttgaaggcgacgcgttgatccgtttcgttgaagatgatggttggattttccagggtaaaggtggctgcgtgggattgcaggctgcatgcggcgaaaagcgatgccaggaatgttttgatacgggaagaacgaaaaagaatatgagagagcatgagtgaattcatcttagggagttgatgatgcggaaggctgataagcggtggttattttgtatggattcgacatatgcaatattatattttgtgattcgagcatggagtgggacgagtcctgttatccggtgggacaagtactagttcgcttgattcatgcctctggtcgtgcgggtggaaaacgagatcgctgaagttgacgttttatttggagtgtgggcgactatggactcgatcggaaatcgatcttcgtttgtcggatcaaattaaccttgaggaatgtctcggccttgcatattgaagatgctgatttttatgcggaaactaaataattatctggaaggcgatgcgagggggatattggtgaggtgcgcttcctcgaaatcgatgaagagggtgtgcgcactaatgcggtaatgacgataaatgatcgagttttggcatgtttgccagatggcatgcgggtgcaatccggggcgagttatatttataggccgaaagcgaatgacaaagttgtcgagccatccaggtcgatcgattcggtgagcggacccgccatgatgcttgttccagccagataggcattgactgtcaggtcggccgtaaaatctctgcctatagcaagactattcggcgacccccaaccttggattatatccgaatgacggacatagatgtccgtgtcgctggtctgtaaagtattaaataatttcgttgcttcgccgtcaacggtgggatttgtcattttaatcgtgtagtagcctagcttgcctgttttgttgaccatgccaaggccgaacgtattcaacgtcctactgtcgtgcgatgaaacgctggcagattcgctgcgattgtctgttaccttgaaagtcatgaaggtgttggcatcgcaatttacggaccaagcttttgtcatttttccgagatcggtatgacccgtgccgctcgttattatcgtcggcaagatctttccgtaatcgtatatgccgttgttcgcaacggagacagtgcagctcggcacgtcaattgtgcccttgaccatgagatccaagtttggtgtatcggcttgaatattagccgaccaaagttgcgcgatgataattgaaattgcgtaaaatgtgatttttggtttcatgggaaatttgatgtatttaaggttggatgggattttgttgaattgggatgattcgttttgtaacgagtgcgatcgtatgtgtggtatccggtacgttggggcgaccgtctcttatgctttttgtgttttaacgagtcgcgggggatagtgggctgtaatcagccgcgaaacacgatcgatgttcgggcgcatcgatcgtgcccgaacatcagtttgagacggttagaggccgaacgcgaaagtcagcgttgcggagccgtccagatcgactgcgtcctttagcgcgccacccatcgtttcggtgccggcgagaaccgcttcgacgaccatgttcgcagtgaaattcttgcctgcgagttgcgtcgcggagctctcgtttgcccacccatgcgtgtagctcgggagcttattcagtttttgatacgtggcgacaactcccgtaccaccgagagacgtgctgggcgttgcatacactcgcgcctttttgccgtctacctgcgcatcttgcatcgtcaccgagtagtaacccaatttgccggtctcgttgaccttacccaggccaaatgccgtgttgtcgccggagctcgccgaatcgctgcgattgtccgtcactttgaagctcatatacgtttcggcatcgcagttcacggtccatgccttttccatcttggcaagcggcgtagtcgccgtgccgctcttgatgagcgtcggagagattttcccatagtcgtacacaccattgtcggcggcaacgacggtacaggccggcacatcaatcgagcccttgacgatgagatcttgagtcggtgcggcagcatgagccgccgtcgtaccgagcatggcgatggacagcgcgagaagagaggttttcagcttcat

SEQ ID NO: 117 - DNA, 2385 bp, Burkholderia pseudomallei
gtgtacgcaagacggttggtcctgtcgtttagcttgctcctcggcatcgcatggggaagcaacgacgcactcgccgtctcgtcgccttcgcccagcgcggcggttgaattcgacacccaaacgctgattgcgcgaggctattccgccgacattgcaaaattcttcagcggcgattcgcgctttctccccggcaagcaggaggtcacgatcgcactcaacgccaaccagacctacacgacgaacgttcaatttgatgacgacggcgatccgtgcttcgacgccgcgctgttggacacgctgcgactgcgcaaaccatccgatctggccggctgcgccgatcccgaacaaatgtggccggaatttcacatgaaactccaccctgggcagtttcgaatcgacatgaccgtgccggaggaagcattcgacccacggcgacgcggcaacggactgcactccggcggcgaggcggtgttactgaattacgatgtcttcgcgcagcgcttcgatacccggttcggcagcagccattacttacaggcacggctcgagcccggcttcaatatcggaaactggtttttccgtaaccgtggagcgctgacccattcaggcaagggattctcgtatcagttgcaagacacctacctcgcgcgtaccgtcgacgcgtggggggccatcacgcagatcggccagttcacgtccacaggcgaaaatctgggcggcctgccgatgctcggcttccagatcagttcggaccgcttgcaaaatagcgggcaactgaccgttcccatccaaggcatcgcggaaagcaacgcgacggtcgaagtgcgtcagcgcggccgtgtcgtctatcgcacgatcgtcccgcccgggcctttcatgctgtccgacatcggcagcgtcgcgcagaacgccgacctccaaatcgaaatcaccgaacaggacggacgaaagcgacgcttcgagatgcccgtcggcatgggctatgccgacgcgaagcagccgacgacttggcaggccgccatcggccgctatcaggatgcaggcaattccgatcgaacgcgtgcacccgcgatattcgccaccggtgaaatctctttctcaccgctcgacaatctacgcgtgtcgaccgccgcgctcgcggcctccggctatcttcatgcgagcgtccaaggaacgctttcggcgaaatccggagcatggcttgcaagcggagtgcgctattcgcggatggctggcgcgggccaaggatatcagttcgagttacaaggctcgtcccgaatcggaggaaacgtgttcgcatccgcctcatggctgtcgcgctcgattcgctataccacgccagatgtcgcgctcggatcgcttggcgatgcaaccggaaccggtcaaaccaagcattcggcgaacgcgtcgatcaactgggcccatccgcgttggggtagctttacgtatggcctgtcgtacaacagtttcgtcggcgcaccgaactccgtatctcacacgctgagcatcggtcgcaagatcggtcgggcaaacgtcagccttgccgcacaaatcagccccaagagcggcagcagcgtctacgccaacgtgtcgattccgctaggcggtggttcgttgagtgcgcgaaccacgcgctcgcagcggggggatctcacgttcggcacgtcctacggcaatcgattgggacgagacggatcgtattccatcggcgcatcgggcagcaagagcggccagaccgcgaacgcgtccgtcagcatgaggacgggcttcgctcaatttggcgccggcgtggctcagagtacgtccaactcacggtctacctacctgtctgcaagcggaggcatcgcctacgccaatggactattcggcacggcatccagccggatcggggaaacgttcgccatcgtgtcggtcccgaaccaaaagggtttgcgcatcagcgcacctggtggaacgtccatcaccaacccattcggcaccgcagtcatctcttccctgacgccctacacgaaagcgcaactcagactcgatacgcaaagccttcccatgaatgtgcgtctcgacacgaccaccgtcgacatcgggctccgacggggcagcgtgctgacgcgcacgatccatgcagccgaaacgcgacagctcatgttgacgatacgagattcgaccggtgccaccgcaccggtcggcaccaccgtgcttgatgagcaaggccagttcctcggcacgatcatcggagatgggaacttcatcctgaatgacgacgacatcggtaggccgatcaggttgtcgggcgtcaatcgtagcgagtgccgcgtgacctacgaggcacccgcacggttcgatccaaatcagccttatgaagaggcagaaggaacatgcgggtga

SEQ ID NO: 118 - DNA, 711 bp, Burkholderia pseudomallei
atgaattcactcatgctctctcatattctttttcgttcttcccgtatcaaaacattcctggcatcgcttttcgccgcatgcagcctgcaatcccacgcagccacctttaccctggaaaatccaaccatcatcttcaacgaaacggatcaacgcgtcgccttcaacgtcaaaaacgacggcgaaagcccgatgctgctcatcagcaagctcgaagacctcggtgaagacaagctcagtcaacgcttcctgattgcgccgccaatcgcacgtatcgatcctggtcaaagtcagcagatcaatttttcgctcaagaagggagccaagctcgagcacgagtacatgctcaaggcgtcgttcgaaggggtatcgcaaactggccgcggctcgctcgtgatgccggttcgccaggaaatcggctttttggtccaggccggcgcgattcccgtttccaaaaccccttgggataacttgaagctcagcgtcaaggataagcaattgactctcagcaatccgagcgcgcacgtgatccggcttgcgccgagcgtcaaactgctgcccacgaacaagatggtcgcgcttgcgcatgcttatctgatgccgggcgaatcacggactgtcgaaatcgacgacgccaccgacaaaatcgagatcgttccactcagccgctacggcctcgtactgcccgcagtcacgatgccggtccagcattaa

SEQ ID NO: 119 - DNA, 648 bp, Burkholderia pseudomallei
atgaaaccaaaaatcacattttacgcaatttcaattatcatcgcgcaactttggtcggctaatattcaagccgatacaccaaacttggatctcatggtcaagggcacaattgacgtgccgagctgcactgtctccgttgcgaacaacggcatatacgattacggaaagatcttgccgacgataataacgagcggcacgggtcataccgatctcggaaaaatgacaaaagcttggtccgtaaattgcgatgccaacaccttcatgactttcaaggtaacagacaatcgcagcgaatctgccagcgtttcatcgcacgacagtaggacgttgaatacgttcggccttggcatggtcaacaaaacaggcaagctaggctactacacgattaaaatgacaaatcccaccgttgacggcgaagcaacgaaattatttaatactttacagaccagcgacacggacatctatgtccgtcattcggatataatccaaggttgggggtcgccgaatagtcttgctataggcagagattttacggccgacctgacagtcaatgcctatctggctggaacaagcatcatggcgggtccgctcaccgaatcgatcgacctggatggctcgacaactttgtcattcgctttcggcctataa

SEQ ID NO: 120 - DNA, 648 bp, Burkholderia pseudomallei; identical to SEQ ID NO: 119

SEQ ID NO: 121 - DNA, 657 bp, Burkholderia pseudomallei
atgaagctgaaaacctctcttctcgcgctgtccatcgccatgctcggtacgacggcggctcatgctgccgcaccgactcaagatctcatcgtcaagggctcgattgatgtgccggcctgtaccgtcgttgccgccgacaatggtgtgtacgactatgggaaaatctctccgacgctcatcaagagcggcacggcgactacgccgcttgccaagatggaaaaggcatggaccgtgaactgcgatgccgaaacgtatatgagcttcaaagtgacggacaatcgcagcgattcggcgagctccggcgacaacacggcatttggcctgggtaaggtcaacgagaccggcaaattgggttactactcggtgacgatgcaagatgcgcaggtagacggcaaaaaggcgcgagtgtatgcaacgcccagcacgtctctcggtggtacgggagttgtcgccacgtatcaaaaactgaataagctcccgagctacacgcatgggtgggcaaacgagagctccgcgacgcaactcgcaggcaagaatttcactgcgaacatggtcgtcgaagcggttctcgccggcaccgaaacgatgggtggcgcgctaaaggacgcagtcgatctggacggctccgcaacgctgactttcgcgttcggcctctaa

SEQ ID NO: 122 - DNA, 24 bp, Artificial
  Synthetic
tgacccattcaggcaagggattct

SEQ ID NO: 123 - DNA, 24 bp, Artificial
  Synthetic
tccgtcctgttcggtgatttcgat

SEQ ID NO: 124 - DNA, 24 bp, Artificial
  Synthetic
gtcgatttcggctgcgaaacaaca

SEQ ID NO: 125 - DNA, 24 bp, Artificial
  Synthetic
atgccgtcgcaaccattgatgatg

(19) United States
(12) Patent Application Publication
(10) Pub. No.: US 2010/0136555 A1
(43) Pub. Date: Jun. 3, 2010

(54) Burkholderia Pseudomallei Diagnostic Genetic Elements that Predict Mortality in Melioidosis

(75) Inventors: Raymond Auerbach, New Haven, CT (US); Bartholomew John Currie, Darwin (AU); Apichai Tuanyok, Flagstaff, AZ (US); Dave Wagner, Flagstaff, AZ (US); Paul Keim, Flagstaff, AZ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Arizona Board of Regents, A body corporate acting for and on behalf of Northern Arizona Univ., Scottsdale, AZ (US)

(21) Appl. No.: 12/598,315

(22) PCT Filed: Jul. 7, 2008
(86) PCT No.: PCT/US08/69326

§ 371 Date: Feb. 15, 2010

Related U.S. Application Data
(60) Provisional application No. 60/948,324, filed on Jul. 6, 2007.

(51) Int. Cl. C12Q 1/68

(52) U.S. Cl. 435/6

(57) ABSTRACT

The present invention provides methods for predicting the likelihood of mortality from melioidosis and detecting the presence of Burkholderia pseudomallei in a test sample.

### CROSS REFERENCE

This application claims priority to U.S. Provisional Patent Application Ser. No. 60/948,324 filed Jul. 6, 2007, incorporated by reference herein in its entirety.

### STATEMENT OF GOVERNMENT SUPPORT

This study was funded by the Pacific Southwest Regional Center of Excellence via subprojects 2005-1593 and 2006-1757 of NIH grant 5 U54 AI065359. The U.S. government owns certain rights in the invention.

### SUMMARY OF THE INVENTION

The present invention provides methods for predicting likelihood of mortality from melioidosis, comprising

(a) analyzing a test sample from a subject suffering from melioidosis for presence or absence of:
i) ten or more contiguous nucleotides of a YLFC gene cluster nucleotide sequence according to nucleotides 153167-158624 of GenBank accession number NC_{—}006351 or complements thereof, and/or
ii) ten or more contiguous nucleotides a BTFC nucleotide sequence according to GenBank accession number EF377328; and

(b) correlating a presence of the ten or more contiguous nucleotides of the BTFC gene cluster, or complements thereof, with a decreased likelihood of subject mortality from melioidosis; and/or;

(c) correlating a presence of the ten or more contiguous nucleotides of the YLFC with an increased likelihood of subject mortality from melioidosis.

The present invention also provides a method for predicting likelihood of mortality from melioidosis, comprising:

(a) analyzing a test sample from a subject suffering from melioidosis for presence or absence of:
i) one or more BTFC polypeptides as set forth in any of SEQ ID NOs: 1-55, or antigenic fragments thereof
ii) one or more YLFC polypeptides as set forth in any of SEQ ID NOs: 56-59, or antigenic fragments thereof.

(b) correlating a presence of the one or more BTFC polypeptides with a decreased likelihood of subject mortality from melioidosis; and/or;

(c) correlating a presence of the one or more YLFC polypeptides with an increased likelihood of subject mortality from melioidosis.

### BRIEF DESCRIPTION OF THE FIGURES

FIG. 1: Genomic diversity between B. pseudomallei strains K96243 and 305 compared to the homologous region in the genome of B. thailandensis E264. An event of horizontal gene transfer caused the ancestral B. thailandensis-like flagella and chemotaxis gene clusters (BTFC) to be replaced by the acquired Yersinia-like fimbriae genes (BPSS0120-0123), as observed in strain K96243. The BTFC remains in strain 305 and has 91-94% nucleotide similarity to B. thailandensis E264.

FIG. 2: Multiplex SYBR-Green real-time PCR assay targeting genes btfc-orf18 and BPSS0120. This assay divides B. pseudomallei into two distinct groups. Strain 305 was the positive control strain for Group1, whereas strain K96243 was the positive control for Group2. (A) Derivative dissociation curves of two different PCR amplicons for gene btfc-orf18, melted at 80.0° C., and gene BPSS0120, melted at 88.0° C. NTC, no-template control. (B) PCR amplicons sizes 115 by and 350 by resolved by agarose gel electrophoresis for genes btfc-orf18 and BPSS0120, respectively. Lanes 1-4 contain: strain 305, strain K96243, no-template control (NTC), and 100 by DNA ladder, respectively.

FIG. 3: Countries of origin for isolates in B. pseudomallei Groups 1 and 2. Group 1 strains were dominant in Australia, whereas Group 2 strains were dominant in Thailand.

FIG. 4: Mortality rates after exposure to B. pseudomallei in the Darwin prospective melioidosis study. Statistics were preformed using a Chi squared test. Indigenous refers to Indigenous Australian (including Aboriginal or Torres Strait Islander) as self identified. G1=Group BTFC, G2=Group YLF

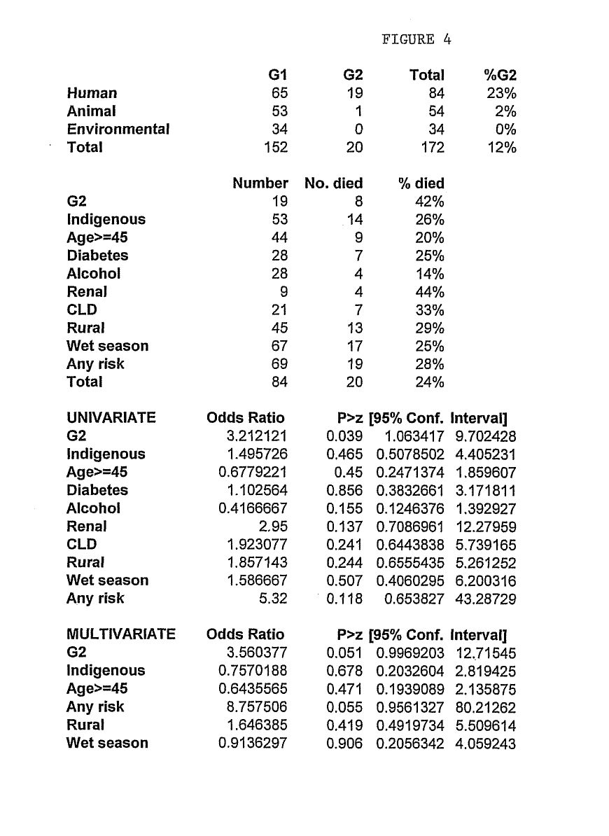

### DETAILED DESCRIPTION OF THE INVENTION

In a first aspect, the present invention provides method for predicting likelihood of mortality from melioidosis, comprising

(a) analyzing a test sample from a subject suffering from melioidosis for presence or absence of:
i) ten or more contiguous nucleotides of a Yersinia-like fimbriae cluster (YLFC) nucleotide sequence according to nucleotides 153167-158624 of GenBank accession number NC_{—}006351 or complements thereof, and/or
ii) ten or more contiguous nucleotides of a Burkholderia thailandensis-like flagella and chemotaxis (BTFC) gene cluster nucleotide sequence according to GenBank accession number EF377328; and

(b) correlating a presence of the ten or more contiguous nucleotides of the BTFC gene cluster, or complements thereof, with a decreased likelihood of subject mortality from melioidosis; and/or;

(c) correlating a presence of the ten or more contiguous nucleotides of the YLFC with an increased likelihood of subject mortality from melioidosis.

Further to this aspect, the present invention provides a method for predicting likelihood of mortality from melioidosis, comprising:

(a) analyzing a test sample from a subject suffering from melioidosis for presence or absence of:
i) one or more BTFC polypeptides as set forth in any of SEQ ID NOs:1-55, or antigenic fragments thereof
ii) one or more YLFC polypeptides as set forth in any of SEQ ID NOs: 56-59, or antigenic fragments thereof;

(b) correlating a presence of the one or more BTFC polypeptides with a decreased likelihood of subject mortality from melioidosis; and/or;

(c) correlating a presence of the one or more YLFC polypeptides with an increased likelihood of subject mortality from melioidosis.

Melioidosis, a tropical disease characterized by severe pulmonary distress with frequent progression to septicemia and death, is a significant cause of mortality and morbidity of people in Southeast Asia and Northern Australia, and is caused by a gram-negative, soil dwelling bacterium named Burkholderia pseudomallei. This pathogen also is a potential biological threat agent and is classified as a Category B Select Agent [9] in the United States. The genome of B. pseudomallei shares a high degree of similarity with that of the closely related, B. thailandensis, although the latter species is free-living, non-pathogenic, and found primarily in Thailand.

As disclosed in more detail below, the inventors have identified a Burkholderia thailandensis-like flagella and chemotaxis (BTFC) gene cluster in B. pseudomallei strain 305 from Australia. The homologous genomic location in B. pseudomallei reference strain K96243 from Thailand has been replaced by a horizontally-acquired Yersinia-like fimbriae cluster (YLFC). These alternate genomic states define two distinct groups within B. pseudomallei. These two gene clusters are mutually exclusive; and the inventors have discovered that presence of the BTFC gene cluster correlates with a decreased likelihood of subject mortality, while presence of the YLFC correlates with an increased likelihood of subject mortality. Increased or decreased likelihood of mortality therefore is defined as a statistically relevant increase or decrease in mortality rate.

Determination of mortality rates is beneficial to the application of treatment regimen. Melioidosis has historically been treated with a prolonged course of antibiotics, beginning with at least two weeks of IV therapy. However, less severe disease from the less pathogenic strains may be adequately managed without the initial IV antibiotics. Thus, in a further embodiment, the method further comprises making a treatment decision based, at least in part, on detection of the BTFC gene cluster vs. the YLFC gene cluster, wherein detection of the BTFC gene cluster vs. the YLFC is taken into consideration in treating the subject with an antibiotic regimen. The present invention therefore provides a platform to determine which courses of treatment are most beneficial as well as cost efficient.

In various embodiments of this first aspect of the invention, the method may be used to assess the origins of B. pseudomallei strains in outbreak scenarios (e.g. a bio-threat event) or in cases diagnosed in non-endemic locations (e.g. in travelers becoming sick after having returned from melioidosis endemic locations).

All B. pseudomallei strains tested to date by the inventors have contained only one of the two gene clusters and neither the BTFC gene cluster nor the YLFC gene cluster are found in the closely related Burkholderia mallei and Burkholderia thailandensis. Thus in a second aspect, the present invention provides methods for detecting the presence of Burkholderia pseudomallei in a sample, comprising

(a) analyzing a test sample from a subject suspected of suffering from a Burkholderia infection for the presence or absence of:
i) ten or more contiguous nucleotides of a Yersinia-like fimbriae cluster (YLFC) nucleotide sequence according to nucleotides 153167-158624 of GenBank accession number NC_{—}006351 or complements thereof, and/or
ii) ten or more contiguous nucleotides of a Burkholderia thailandensis-like flagella and chemotaxis (BTFC) gene cluster nucleotide sequence according to GenBank accession number EF377328; and

(b) correlating a presence of the ten or more contiguous nucleotides of the BTFC gene cluster, or complements thereof, or the ten or more contiguous nucleotides of the YLFC with the presence of Burkholderia Pseudomallei in the test sample.

Further to this aspect, the invention provides a method for detecting the presence of Burkholderia. pseudomallei in a sample, comprising

(a) analyzing a test sample from a subject suspected of suffering from a Burkholderia infection for the presence or absence of:
i) one or more BTFC polypeptides as set forth in any of SEQ ID NOs: 1-55, or antigenic fragments thereof, and/or
ii) one or more contiguous amino acids of a YLFC polypeptide as set forth in any of SEQ ID NOs: 56-59, or antigenic fragments thereof; and

(b) correlating a presence of the one or more BTFC polypeptides, or one or more of the YLFC polypeptides with the presence of Burkholderia. Pseudomallei in the test sample.

The subject can be any host susceptible to infection by Burkholderia pseudomallei, including but not limited to humans, animals that are farmed in the tropics (e.g. goat, sheep, pig) and primates and felines imported from the tropics that may be found for example in zoos. Humans that may be hosts are those for example who work in rice paddies and those who are exposed due to environmental perturbation including, but not limited to earthworks (i.e., activities that disturb soil), cyclones, and “Tsunami lung” (necrotizing pneumonia, for example, caused by a near drowning event during a tsunami).

The test sample can be any sample from the subject in which Burkholderia pseudomallei DNA can be found. In one embodiment, B. pseudomallei DNA can be isolated from the test sample, although isolation is not required. In another embodiment, Burkholderia pseudomallei can be cultured from the test sample to increase the amount of DNA available for analysis. In other embodiments, mixed host and B. pseudomallei DNA can be isolated for analysis. Test samples can include, but are not limited to, throat swabs, sputum, urine, blood, sputum, pus, pleural fluid, cerebrospinal fluid, and wound and rectal swabs.

The methods comprise detecting 10 or more contiguous nucleotides of the recited BFTC gene cluster and/or YLFC. In various embodiments, the methods comprise detecting 20, 30, 40, 50, 60, 70, 80, 90, 100, 150, 200, 300, 500, 1000, or more contiguous nucleotides of the recited BFTC gene cluster and/or YLFC. In a further embodiment, the methods comprise detecting one or more full length genes, or all of the genes, encoded in the BFTC gene cluster (one or more of btfc-orf 1-55 as recited in SEQ ID NOS: 60-115) or the YLFC (one or more of YP110141, YP110142, YP110143, and YP110144 as recited in SEQ ID NOS: 116-121).

In one embodiment, the detecting comprises detecting 10, 20, 30, 40, 50, 60, 70, 80, 90, 100, 150, 200, 300, 500, 1000, or more contiguous nucleotides of the btfc-orf18 gene from the BTFC gene cluster, wherein the presence of such contiguous nucleotides is predictive of a decreased likelihood of mortality.

In one embodiment, analyzing a test sample from a subject suffering from melioidosis for presence or absence of BFTC gene cluster and/or YLFC gene cluster can be performed by using a hybridization technique where one or more nucleotide probes complementary to 10 or more nucleotides of the BFTC gene cluster or YLFC gene cluster nucleic acids or complements thereof, and detecting hybridization products. Such detecting can be by any suitable means, including but not limited to in situ hybridization, colony hybridization (for example, on cultured bacterial colonies derived from the test samples), Southern blots, Northern blots, etc. Suitable hybridization conditions for promoting binding of complements while minimizing or inhibiting binding of mismatched probes can be determined by those of skill in the art based on the teachings herein. An example of such conditions involve forming hybridization complexes in 0.2×SSC at 65° C. for a desired period of time, and wash conditions of 0.2×SSC at 65° C. for 15 minutes to remove unbound probe. It is understood that these conditions may be duplicated using a variety of buffers and temperatures. SSC (see, e.g., Sambrook, Fritsch, and Maniatis, in: Molecular Cloning, A Laboratory Manual, Cold Spring Harbor Laboratory Press, 1989) is well known to those of skill in the art, as are other suitable hybridization buffers. It will be apparent to a person having ordinary skill in the art that the stringency of nucleic acid hybridization conditions can be affected or adjusted by other factors, including without limitation and by way of example only, the choice of polymerase used, and the magnesium and other concentration and other composition of the ligase or polymerase buffer.

In another embodiment of the invention, the analyzing comprises contacting nucleic acids in the test sample with one or more primer pairs complementary to nucleotide sequence regions of interest within the BTFC or YLFC gene cluster, or complements thereof, under conditions suitable for amplifying the BTFC or YLFC gene cluster nucleotide regions of interest, and detecting presence or absence of amplification products. Such amplification can be by any suitable technique, including but not limited to polymerase chain reaction techniques. Primers complementary to the BTFC and/or YLFC regions of interest can be determined by those of skill in the art based on the teachings herein, as can suitable conditions for amplification.

In another embodiment of the invention, the analyzing comprises sequencing DNA from a test sample and detecting sequences diagnostic for the BTFC gene cluster or the YLFC. “Nucleotide sequencing” can be performed using any suitable technique, including but not limited to use of standard chain-termination methods in which primers that hybridize to a DNA sample can be fluorescently or radiolabelled, or fluorescently labeling the DNA sample with dideoxynucleotide chain-terminators specific for each nucleotide type.

In a further aspect, the invention provides methods for detecting the presence of one or more of the polypeptides of the invention in a protein sample, comprising providing a protein sample to be screened, contacting the protein sample to be screened with an antibody against one or more of the polypeptides selected from the group consisting of SEQ ID NOs: 1-59, or antigenic fragments thereof and detecting the formation of antibody-antigen complexes. In a preferred embodiment, the methods comprise:

a) providing a protein sample to be screened;

b) contacting the protein sample to be screened with an antibody selective for one or more BTFC and/or YLFC polypeptides disclosed herein, or antigenic fragments thereof, under conditions that promote antibody-antigen complex formation; and

c) detecting the formation of antibody-antigen complexes, wherein the presence of the antibody-antigen complex indicates the presence of a protein comprising or consisting of a sequence selected from the group consisting of SEQ ID NOs: 1-59.

The antibody can be polyclonal or monoclonal. As used herein, the term “protein sample” refers to any sample that may contain the polypeptides of the invention, and fragments thereof, including but not limited to tissues and portions thereof, tissue sections, intact cells, cell extracts, purified or partially purified protein samples, bodily fluids, and nucleic acid expression libraries. Accordingly, this aspect of the present invention may be used to test for the presence of the non-canonical BTFC or YLFC polypeptides disclosed herein in these various protein samples by standard techniques including, but not limited to, immunolocalization, immunofluorescence analysis, Western blot analysis, ELISAs, and nucleic acid expression library screening, (See for example, Sambrook et al, 1989.) In one embodiment, the techniques may determine only the presence or absence of the protein or peptide of interest. Alternatively, the techniques may be quantitative, and provide information about the relative amount of the protein or peptide of interest in the sample. For quantitative purposes, ELISAs are preferred.

Detection of immunocomplex formation between the polypeptides of the invention, and their antibodies or fragments thereof, can be accomplished by standard detection techniques. For example, detection of immunocomplexes can be accomplished by using labeled antibodies or secondary antibodies. Such methods, including the choice of label are known to those ordinarily skilled in the art (Harlow and Lane, Supra). Alternatively, the antibodies can be coupled to a detectable substance. The term “coupled” is used to mean that the detectable substance is physically linked to the antibody. Suitable detectable substances include various enzymes, prosthetic groups, fluorescent materials, luminescent materials and radioactive materials. Examples of suitable enzymes include horseradish peroxidase, alkaline phosphatase, 13-galactosidase, or acetylcholinesterase. Examples of suitable prosthetic-group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of suitable radioactive material include ¹²⁵I, ¹³¹I, ³⁵S or ³H.

The term antibody as used herein is intended to include antibody fragments thereof which are selectively reactive with the polypeptides of the invention, or fragments thereof. Antibodies can be fragmented using conventional techniques, and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

Antibodies can be made by well-known methods, such as described in Harlow and Lane, Antibodies; A Laboratory Manual, Cold Spring Harbor Laboratory, Cold Spring Harbor, N.Y., (1988). In one example, preimmune serum is collected prior to the first immunization. A substantially purified polypeptide of the invention, or antigenic fragments thereof, together with an appropriate adjuvant, are injected into an animal in an amount and at intervals sufficient to elicit an immune response. Animals are bled at regular intervals, preferably weekly, to determine antibody titer. The animals may or may not receive booster injections following the initial immunization. At about 7 days after each booster immunization, or about weekly after a single immunization, the animals are bled, the serum collected, and aliquots are stored at about −20° C. Polyclonal antibodies against the proteins and peptides of the invention can then be purified directly by passing serum collected from the animal through a column to which non-antigen-related proteins prepared from the same expression system without GPBP-related proteins bound.

Monoclonal antibodies can be produced by obtaining spleen cells from the animal (See Kohler and Milstein, Nature 256, 495-497 (1975)). In one example, monoclonal antibodies (mAb) of interest are prepared by immunizing inbred mice with the proteins or peptides of the invention, or an antigenic fragment thereof. The mice are immunized by the IP or SC route in an amount and at intervals sufficient to elicit an immune response. The mice receive an initial immunization on day 0 and are rested for about 3 to about 30 weeks. Immunized mice are given one or more booster immunizations of by the intravenous (IV) route. Lymphocytes, from antibody positive mice are obtained by removing spleens from immunized mice by standard procedures known in the art. Hybridoma cells are produced by mixing the splenic lymphocytes with an appropriate fusion partner under conditions which will allow the formation of stable hybridomas. The antibody producing cells and fusion partner cells are fused in polyethylene glycol at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells and are screened for antibody production by an immunoassay such as solid phase immunoradioassay. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

To generate an antibody response, the polypeptides of the present invention are typically formulated with a pharmaceutically acceptable carrier for parenteral administration. Such acceptable adjuvants include, but are not limited to, Freund's complete, Freund's incomplete, alum-precipitate, water in oil emulsion containing Corynebacterium parvum and tRNA. The formulation of such compositions, including the concentration of the polypeptide and the selection of the vehicle and other components, is within the skill of the art.

The various embodiments of each aspect of the invention are equally suitable for use in other aspects of the invention except where clearly noted otherwise based on the context. Similarly, the various embodiments of each aspect can be combined, except where clearly noted otherwise based on the context.

### Examples

A Burkholderia thailandensis-like flagella and chemotaxis (BTFC) gene cluster was identified in B. pseudomallei strain 305 from Australia. The homologous genomic location in Thailand B. pseudomallei reference strain K96243 has been replaced by a horizontally-acquired Yersinia-like fimbriae cluster. These alternate genomic states define two distinct groups within B. pseudomallei.

A Gram-negative, soil-dwelling bacterium, Burkholderia pseudomallei is the causative agent of melioidosis, a tropical disease that is a significant cause of mortality and morbidity of people in Southeast Asia and Northern Australia (1, 10). This pathogen also is a potential biological threat agent and is classified as a Category B Select Agent [9] in the United States. The genome of B. pseudomallei shares a high degree of similarity with that of the closely related B. thailandensis, although the latter species is free-living, non-pathogenic, and found primarily in Thailand.

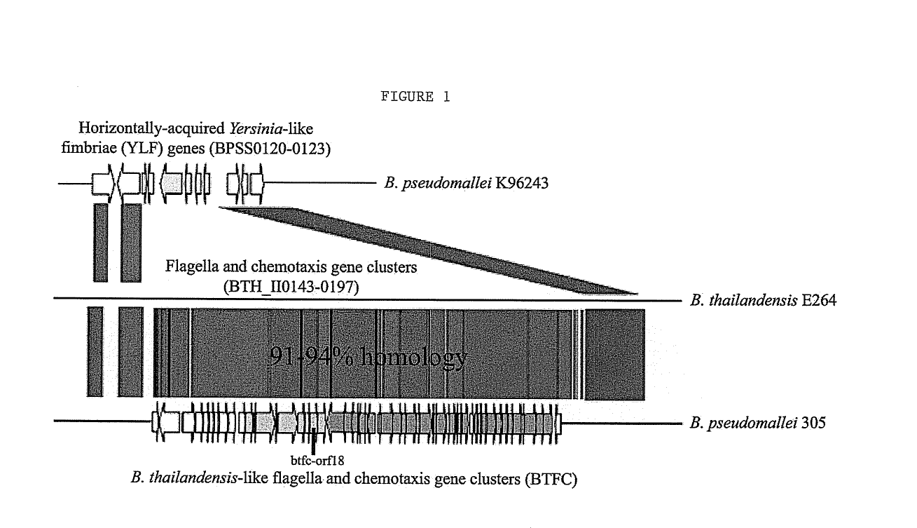

Several recent studies have compared whole genome sequences of B. pseudomallei strains to sequences of strains of B. thailandensis and/or the closely-related species, B. mallei (7, 8, 11). Genomic comparison of B. pseudomallei strain K96243 (6) and B. thailandensis strain E264 revealed high similarity between the two syntenic chromosomes, with most of the differences attributed to the presence of several virulence-related genes in B. pseudomallei that are absent in B. thailandensis (11). These genes include a capsular polysaccharide gene cluster and type III secretion system. Several horizontal gene transfer events are believed to be the mechanisms by which B. pseudomallei obtained some of the virulence-related genes that are absent in B. thailandensis. These events include the replacement of a distinct set of capsular polysaccharide synthesis genes for the ancestral polysaccharide cluster, and the horizontal acquisition of a Yersinia-like fimbriae cluster by B. pseudomallei resulting in the replacement of an ancestral flagella biosynthesis cluster (7, 11). The latter mechanism is illustrated in FIG. 1, which indicates that there is genomic variability between B. pseudomallei K96243 and B. thailandensis E264 at this particular region on the smaller of the two chromosomes.

Here we show that the acquisition of the Yersinia-like fimbriae cluster is not universal for all B. pseudomallei strains. Indeed, in many strains the ancestral B. thailandensis-like flagella biosynthesis gene clusters remain largely intact. We first identified this ancestral state in the genome sequences of four different B. pseudomallei strains: 668 (GenBank accession #AAHU00000000), 1655 (AAHR00000000), and 406e (AAMM00000000), which were sequenced by TIGR (www.tigr.org); and strain 305, which was sequenced at the DOE Joint Genome Institute (JGI). With the exception of strain 406e, these strains originated from patients from the Northern of Australia as part of the Darwin prospective melioidosis study (3).

To annotate this diverse region in the genome sequence of the strain 305, we used the software packages Glimmer3 and GeneMark to predict open reading frames (ORFs) and then aligned predicted ORFs against NCBI's “non-redundant” protein-database using blastx. This annotation identified 55 predicted ORFs that were clustered into at least two groups, including genes for flagella biosynthesis and genes for chemotaxis biosynthesis proteins. These genes are unique and different from other flagella biosynthesis and chemotaxis protein genes in the B. pseudomallei 305 genome. We have named this region the B. thailandensis-like flagella and chemotaxis gene clusters, or BTFC, according to GenBank accession # EF377328. Genomic comparison between the BTFC of B. pseudomallei 305 and the homologous ancestral region of B. thailandensis E264 was performed using the software packages BioEdit (Ibis Therapeutics) and Artemis Comparison Tool (Sanger Institute). As shown in FIG. 1, this comparison revealed approximately 91-94% nucleotide similarity between the two species. Within B. pseudomallei, the nucleotide sequence of the BTFC region was highly conserved, with as much as 99% similarity among the genome sequences of strains 406e, 1655, and 668 (data not shown).

To investigate the prevalence of BTFC and the event of the horizontal acquisition of the fimbrial genes among diverse isolates of B. pseudomallei, we developed a multiplex real-time PCR assay using SYBR-Green as a fluorescence dye reporter. Gene BPSS0120 (fimbriae usher protein) was utilized as a marker for the horizontal acquisition of the Yersinia-like fimbrial gene region, whereas gene btfc-orf18 was utilized as a representative marker for BTFC. The PCR primers that we used in this study are as follows:

(SEQ ID NO: 122)

BPSS0120_forward:
5′- TGA CCC ATT CAG GCA AGG GAT TCT -3′,

(SEQ ID NO: 123)

BPSS0120_reverse:
5′- TCC GTC CTG TTC GGT GAT TTC GAT -3′,

(SEQ ID NO: 124)

btfc-orf18_forward:
5′- GTC GAT TTC GGC TGC GAA ACA ACA -3′,
and

(SEQ ID NO: 125)

btfc-orf18_reverse:
5′- ATG CCG TCG CAA CCA TTG ATG ATG -3′.

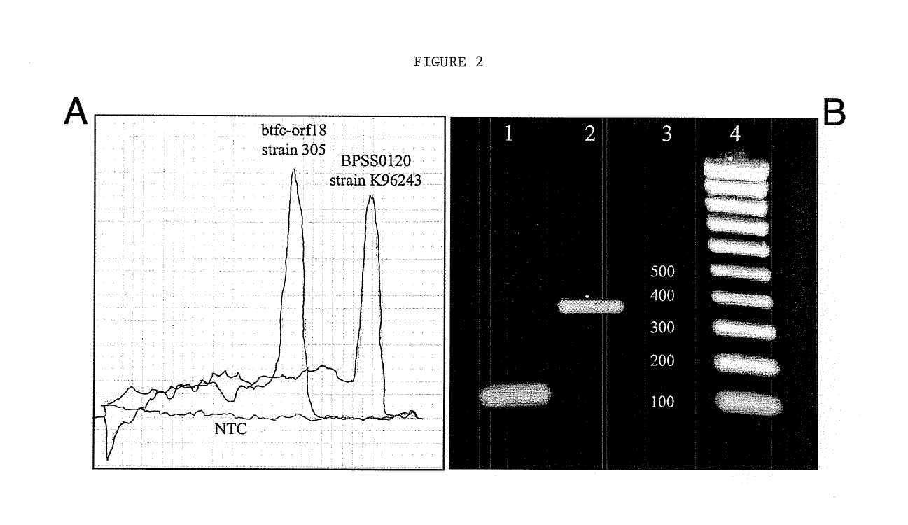

We designed primer btfc-orf18_reverse so it differed from the nucleotide sequence of B. thailandensis E264 at eight nucleotides. This resulted in amplification of the btfc-orf18 amplicon for only B. pseudomallei. The assay was conducted in 10 μL reactions containing: 1×SYBR master mix (Applied Biosystem Inc.), 0.3 μM of each PCR primer, and 0.1-1.0 ng of DNA template. The reactions were performed on an ABI7900HT Sequence Detection System (Applied Biosystem Inc.) utilizing 40 cycles. Each cycle contained 2 steps: denaturation at 95° C. for 15 sec and annealing at 60° C. for 30 sec. PCR products were further analyzed by melting continuously from 60° C. to 95° C. to generate a dissociation curve. Melting temperatures of PCR amplicons for genes btfc-orf18 and BPSS0120 were constant at 80.0° C. and 88.0° C., respectively (see FIG. 2a). We used this assay to analyze DNA templates from a total of 602 diverse B. pseudomallei strains isolated from clinical and environmental situations. DNA from strains K96243 and 305 were used as positive controls for genes BPSS0120 and btfc-orf18, respectively. A collection of 580 strains from Australia (234), Thailand (314) and other geographic origins (32) were examined for the Yersinia-like fimbrial gene region or the btfc-orf18. Each of 580 DNA templates produced only a single PCR amplicon: either 350 by for the BPSS0120 gene, or 115 by for the btfc-orf18 (see FIG. 2b). This indicates that all B. pseudomallei strains examined had only a single gene pattern, either the ancestral BTFC, or the acquired Yersinia-like fimbrial genes. As such, we used this criterion to differentiate B. pseudomallei into 2 distinct groups. Group 1 strains contained the ancestral BTFC, whereas Group 2 strains contained the Yersinia-like fimbrial genes.

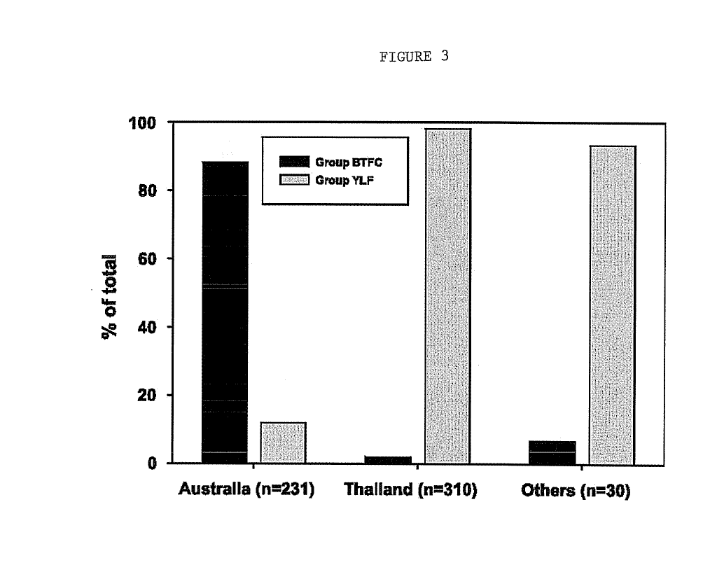

An examination of the countries of origin for the 580 strains revealed differences in the geographic distributions of the two types (FIG. 3). Group 1 is common in Australia (208/234; 89%) but rare in Thailand (7/314; 2%) and other countries (3/32; 9%; Ecuador, n=1; unknown, n=2). In contrast, Group 2 was predominant among the isolates from Thailand (307/314; 98%) and other countries (29/32; 91%; Bangladesh, n=1; Fiji, n=1; Indonesia, n=1; Kenya, n=1; Madagascar, n=1; Malaysia, n=1; Pakistan, n=1; Singapore, n=3; Vietnam, n=1; unknown, n=11) but relatively rare in Australia (26/234; 11%). The replacement of the BTFC genes by the Yersinia-like fimbrial genes may have been a single event during the evolutionary process of B. pseudomallei that conferred different phenotypes to Groups 1 and 2. Although the nature of the phenotypic differences between the two groups may not be immediately obvious, traits that may be affected include environment adaptability and pathogenicity. For example, the acquired Yersinia-like fimbrial genes may actually allow B. pseudomallei to infect novel hosts or to infect existing hosts more effectively, which may allow Group 2 strains to spread more efficiently.

Potential differences in pathogenicity between Group 1 and Group 2 strains offer one explanation for the distinct geographic distributions of these two groups. If the Yersinia-like fimbrial genes offer a fitness advantage, Group 2 could represent a significant expansion of the distribution and global disease-causing potential of B. pseudomallei. The dominance of Group 2 in Thailand is consistent with either differential regional selection for this gene set, or a genetic bottleneck associated with a founder event with these strains. Expansion of this particular type into Thailand via an infected host would explain its dominance in that region. If the genomic replacement event originated in Australia and offered a pathogenic advantage to the Group 2 strains, it suggests that those strains were subsequently introduced to Thailand and elsewhere via a host-mediated dispersal event. Multi-locus sequence typing has shown separation between Australian and That strains of B. pseudomallei (2). In that previous study, it was suggested that B. pseudomallei may possibly have originated in Australia and been propagated through animal migration during the Miocene period around 15 million years ago, when a land bridge joined the Australia-New Guinea continent and southeast Asia. Adaptation to specific ecological niches may also explain the distinct geographic distributions of these two groups. If, as we suggest above, the genomic replacement has only occurred once in B. pseudomallei and that event occurred in Australia, then distinct and occupied niches coupled with balancing selection (e.g. soil versus hosts) would have preserved both groups in Australia. In Australia, the Group 1 strains were already present in the environment and would not be replaced by the Group 2 strains, which still emerge to cause disease. In other words, the Yersinia-like fimbrial genes may offer a host disease adaptation that does not confer additional fitness in the environment. The rare occurrence of Group 1 strains in Thailand may be due to a limited introduction of those strains to that region. Alternatively, their rarity in Thailand may be due to niche competition with B. thailandensis, which is common in soil in Thailand but thought to be rare or absent from Australia. This lack of competition from B. thailandensis may explain why Group 1 B. pseudomallei strains are so prevalent in Australia.

Other studies of genomic diversity among strains of B. pseudomallei have been performed using suppression subtractive hybridization (4, 5) and comparative genomic hybridization (7, 9). These studies demonstrated variability in genomic islands and prophages but did not analyze these differences across populations or geographic regions. In this study, we have identified genomic differences that are highly correlated with specific geographic regions, findings that are significant and provide potential insights into the evolutionary past of B. pseudomallei. Clearly, other genomic differences need to be analyzed across large strain panels to understand their potential importance to the biology of this pathogen.

### REFERENCES

1. Cheng, A. C., and B. J. Currie. 2005. Melioidosis: Epidemiology, pathophysiology, and management. Clin. Microbiol. Rev. 18:383-416.
2. Cheng, A. C., D. Godoy, M. Mayo, D. Gal, B. G. Spratt, and B. J. Currie. 2004. Isolates of Burkholderia pseudomallei from northern Australia are distinct by multilocus sequence typing, but strain types do not correlate with clinical presentation. J. Clin. Microbiol. 42:5477-5483.
3. Currie, B. J., D. A. Fisher, D. M. Howard, J. N. Burrow, D. Lo, S. Selva-Nayagam, N. M. Anstey, S. E. Huffam, P. L. Snelling, P. J. Marks, D. P. Stephens, G. D. Lum, S. P. Jacups, and K. V. L. 2000. Endemic melioidosis in tropical northern Australia: A 10-year prospective study and review of the literature. Clin. Infect. Dis. 31:981-986.
4. DeShazer, D. 2004. Genomic diversity of Burkholderia pseudomallei clinical isolates: Subtractive hybridization reveals a Burkholderia mallei-specific prophage in B. pseudomallei 1026b. J. Bacteriol. 186:3938-3950.
5. Duangsonk, K., D. Gal, M. Mayo, C. A. Hart, B. J. Currie, and C. Winstanley. 2006. Use of a variable amplicon typing scheme reveals considerable variation in the accessory genomes of isolates of Burkholderia pseudomallei. J. Clin. Microbiol. 44:1323-1334.
6. Holden, M. T. G., R. W. Titball, S. J. Peacock, A. M. Cerdeno-Tarraga, T. Atkins, L. C. Crossman, T. Pitt, C. Churcher, K. Mungall, S. D. Bentley, M. Sebaihia, N. R. Thomson, N. Bason, I. R. Beacham, K. Brooks, K. A. Brown, N. F. Brown, G. L. Challis, I. Cherevach, T. Chillingworth, A. Cronin, B. Crossett, P. Davis, D. DeShazer, T. Feltwell, A. Fraser, Z. Hance, H. Hauser, S. Holroyd, K. Jagels, K. E. Keith, M. Maddison, S. Moule, C. Price, M. A. Quail, E. Rabbinowitsch, K. Rutherford, M. Sanders, M. Simmonds, S. Songsivilai, K. Stevens, S. Tumapa, M. Vesaratchavest, S. Whitehead, C. Yeats, B. G. Barrell, P. C. F. Oyston, and J. Parkhill. 2004. Genomic plasticity of the causative agent of melioidosis, Burkholderia pseudomallei. PNAS 101:14240-14245.
7. Kim, H. S., M. Schell, Y. Yu, R. Ulrich, S. Sarria, W. Nierman, and D. DeShazer. 2005. Bacterial genome adaptation to niches: Divergence of the potential virulence genes in three Burkholderia species of different survival strategies. BMC Genomics 6:174.
8. Monastyrskaya, G., A. Fushan, I. Abaev, O. Filyukova, M. Kostina, E. Pecherskih, and E. Sverdlov. 2004. Genome-wide comparison reveals great inter- and intraspecies variability in B. pseudomallei and B. mallei pathogens. Res. Microbiol. 155:781-793.
9. Ou, K., C. Ong, S. Y. Koh, F. Rodrigues, S. H. Sim, D. Wong, C. H. Ooi, K. C. Ng, H. Jikuya, C. C. Yau, S. Y. Soon, D. Kesuma, M. A. Lee, and P. Tan. 2005. Integrative genomic, transcriptional, and proteomic diversity in natural isolates of the human pathogen Burkholderia pseudomallei. J. Bacteriol. 187:4276-4285.
10. White, N. J. 2003. Melioidosis. Lancet 361:1715.
11. Yu, Y., H. S. Kim, H. Chua, C. Lin, S. Sim, D. Lin, A. Derr, R. Engels, D. DeShazer, B. Birren, W. Nierman, and P. Tan. 2006. Genomic patterns of pathogen evolution revealed by comparison of Burkholderia pseudomallei, the causative agent of melioidosis, to avirulent Burkholderia thailandensis. BMC Microbiol. 6:46.

We claim:

1. A method for predicting likelihood of mortality from melioidosis, comprising
(a) analyzing a test sample from a subject suffering from melioidosis for presence or absence of:
i) ten or more contiguous nucleotides of a YLFC nucleotide sequence according to nucleotides 153167-158624 of GenBank accession number NC_{—}006351 or complements thereof, and/or
ii) ten or more contiguous nucleotides of a BTFC gene cluster nucleotide sequence according to GenBank accession number EF377328; and
(b) correlating a presence of the ten or more contiguous nucleotides of the BTFC gene cluster, or complements thereof, with a decreased likelihood of subject mortality from melioidosis; and/or;
(c) correlating a presence of the ten or more contiguous nucleotides of the YLFC with an increased likelihood of subject mortality from melioidosis.

2. A method for detecting the presence of Burkholderia. pseudomallei in a sample, comprising
(a) analyzing a test sample from a subject suspected of suffering from a Burkholderia infection for the presence or absence of:
i) ten or more contiguous nucleotides of a Yersinia-like fimbriae cluster (YLFC) nucleotide sequence according to nucleotides 153167-158624 of GenBank accession number NC_{—}006351 or complements thereof, and/or
ii) ten or more contiguous nucleotides of a Burkholderia thailandensis-like flagella and chemotaxis (BTFC) gene cluster nucleotide sequence according to GenBank accession number EF377328; and
(b) correlating a presence of the ten or more contiguous nucleotides of the BTFC gene cluster, or complements thereof, or the ten or more contiguous nucleotides of the YLFC with the presence of Burkholderia pseudomallei in the test sample.

3. The method of claim 1 or 2, wherein the analyzing comprises
(a) contacting nucleic acids in the test sample with one or more primer pairs complementary to nucleotide sequence regions of interest within the BTFC gene cluster, or complements thereof, under conditions suitable for amplifying the BTFC gene cluster nucleotide regions of interest, and
(b) detecting presence or absence of amplification products.

4. The method of claim 1 or 2, wherein the analyzing comprises
(a) contacting nucleic acids in the test sample with one or more primer pairs complementary to nucleotide sequence regions of interest within the YLFC, or complements thereof, under conditions suitable for amplifying the YLFC nucleotide regions of interest, and
(b) detecting presence or absence of amplification products.

5. The method of claim 1 or 2 wherein the analyzing comprises
(a) contacting nucleic acids in the test sample with one or more hybridization probes complementary to one or more nucleotide sequence regions of interest within the BTFC gene cluster, or complements thereof, under high stringency hybridization conditions, and
(b) detecting presence or absence of hybridization products.

6. The method of claim 1 or 2, wherein the analyzing comprises
(a) contacting nucleic acids in the test sample with one or more hybridization probes complementary to one or more nucleotide sequence regions of interest within the YLFC, or complements thereof, under high stringency hybridization conditions, and
(b) detecting presence or absence of hybridization products.

7. The method of claim 1, wherein the analyzing comprises analyzing a test sample from a subject suffering from melioidosis for presence or absence of:
i) 100 or more contiguous nucleotides of a YLFC nucleotide sequence according to nucleotides 153167-158624 of GenBank accession number NC_{—}006351 or complements thereof, and/or
ii) 100 or more contiguous nucleotides of a BTFC gene cluster nucleotide sequence according to GenBank accession number EF377328; and
the correlating comprises correlating a presence of the 100 or more contiguous nucleotides of the BTFC gene cluster, or complements thereof, with a decreased likelihood of subject mortality from melioidosis; and/or correlating a presence of the 100 or more contiguous nucleotides of the YLFC with an increased likelihood of subject mortality from melioidosis.

8. The method of claim 1 wherein the analyzing comprises analyzing a test sample from a subject suspected of suffering from a Burkholderia infection for the presence or absence of:
i) 100 or more contiguous nucleotides of a Yersinia-like fimbriae cluster (YLFC) nucleotide sequence according to nucleotides 153167-158624 of GenBank accession number NC_{—}006351 or complements thereof, and/or
ii) 100 or more contiguous nucleotides of a Burkholderia thailandensis-like flagella and chemotaxis (BTFC) gene cluster nucleotide sequence according to GenBank accession number EF377328; and
the correlating comprises correlating a presence of the 100 or more contiguous nucleotides of the BTFC gene cluster, or complements thereof, or the 100 or more contiguous nucleotides of the YLFC with the presence of Burkholderia pseudomallei in the test sample.

9. A method for predicting likelihood of mortality from melioidosis, comprising
(a) analyzing a test sample from a subject suffering from melioidosis for presence or absence of:
i) one or more YLFC polypeptides as set forth in any of SEQ ID NOs: 56-59, or antigenic fragments thereof
ii) one or more BTFC polypeptides as set forth in any of SEQ ID NOs: 1-55, or antigenic fragments thereof
(b) correlating a presence of the one or more YLFC polypeptides with an increased likelihood of subject mortality from melioidosis; and/or;
(c) correlating a presence of the one or more BTFC polypeptides with a decreased likelihood of subject mortality from melioidosis.

10. A method for detecting the presence of Burkholderia. pseudomallei in a sample, comprising
(a) analyzing a test sample from a subject suspected of suffering from a Burkholderia infection for the presence or absence of:
i) one or more YLFC polypeptides as set forth in any of SEQ ID NOs: 56-59, or antigenic fragments thereof, and/or
ii) one or more BTFC polypeptides as set forth in any of SEQ ID NOs: 1-55, or antigenic fragments thereof; and
(b) correlating a presence of the one or more YLFC polypeptides, or the one or more BTFC polypeptides with the presence of Burkholderia pseudomallei in the test sample.

11. The method of claim 9 or 10, wherein the analyzing comprises antibody detection of the one or more YLFC polypeptides and/or the one or more BTFC polypeptides.
